(19) United States
(12) Patent Application Publication
(10) Pub. No.: US 2022/0183969 A1
(43) Pub. Date: Jun. 16, 2022

(54) OCULAR INSERT CONTAINING A GLUCOCORTICOID

(71) Applicant: Ocular Therapeutix, Inc., Bedford, MA (US)

(72) Inventors: Charles D. Blizzard, Nashua, NH (US); Ankita Desai, Reading, MA (US); Michael Goldstein, Cambridge, MA (US)

(21) Appl. No.: 17/549,307

(22) Filed: Dec. 13, 2021

Related U.S. Application Data
(63) Continuation-in-part of application No. PCT/US21/41761, filed on Jul. 15, 2021.
(60) Provisional application No. 63/181,720, filed on Apr. 29, 2021.
(60) Provisional application No. 63/124,176, filed on Dec. 11, 2020.

(51) Int. Cl. A61K 9/00; A61K 31/573

(52) U.S. Cl. CPC A61K 9/0051; A61K 31/573

(57) ABSTRACT

In certain embodiments, the invention relates to a sustained release biodegradable intracanalicular insert containing a glucocorticoid dispersed in a hydrogel for the treatment of dry eye disease.

### CROSS-REFERENCE TO RELATED APPLICATIONS

The present invention claims priority to U.S. Provisional Application Ser. No. 63/124,176 filed Dec. 11, 2020, to U.S. Provisional Application Ser. No. 63/181,720 filed Apr. 29, 2021, and to PCT/US21/41761 filed Jul. 15, 2021, which are all hereby incorporated by reference herein.

### TECHNICAL FIELD

The present invention relates to the treatment of dry eye disease (DED), and in certain embodiments to the acute treatment of DED or of episodic flares of DED. According to certain embodiments of the present invention, DED is treated by administering a biodegradable insert into the superior and/or inferior canaliculus of the eye, wherein the insert provides sustained release of a glucocorticoid such as dexamethasone.

### BACKGROUND

Dry eye disease (DED), also known as keratoconjunctivitis sicca (KCS) (and also simply referred to as “dry eye”) is among the most frequently encountered ocular morbidities. A huge number of patients who currently visit ophthalmic clinics report symptoms of dry eye, making it a growing public health problem and among the most common conditions seen by eye care practitioners. Prevalence increases significantly with age and female sex.

DED is a multifactorial disorder of the tears and ocular surface characterized by symptoms of dryness, irritation, burning, stinging, grittiness, foreign body sensation, tearing, and ocular fatigue. Although the pathogenesis of DED is not fully understood, it is recognized that inflammation has a prominent role in the development and propagation of this debilitating condition. Factors that adversely affect tear film stability and osmolarity can induce ocular surface damage and initiate an inflammatory cascade that activates innate and adaptive immune responses. These immunoinflammatory responses lead to further ocular surface damage and the development of a self-perpetuating inflammatory cycle. For instance, inflammation of the ocular surface results in a reduction of tear production, which further deteriorates the mentioned conditions. In humans, dry eye was found to be associated with the presence of conjunctival T-cells and elevated levels of inflammatory cytokines in the tears compared with controls, supportive of inflammation as a driving source of the disorder.

DED is thought to be a chronic state, with episodic flares encompassing rapid onset of symptoms or symptom worsening, highly affecting daily life of patients. Several pharmacological therapies for DED have been explored and include a step-wise approach starting with over the counter lubricants and artificial tear replacements (delivered as eye drops), progressing to topical anti-inflammatory therapy and lacrimal occlusion using punctal or intracanalicular plugs for tear retention. Plugs often consist of collagen, acrylic polymers or silicon. Although plugs have been shown to be effective in patients with DED, plugs are sometimes lost (i.e., show poor retention) and may even migrate into the nasolacrimal duct, where they may be the cause of inflammation or other pathological conditions (cf. Fezza, et al. Study Raises Concern Over Plug, Review of Ophthalmology, 2011).

DED is currently short-term treated with topical glucocorticoids such as fluorometholone (FML), loteprednol etabonate (Alrex® and Lotemax®), and prednisolone acetate (PRED MILD®), as well as with cyclosporine (Restasis®) and lifitegrast (Xiidra®), all of these actives delivered in the form of ophthalmic drops. EYSUVIS™ (Kala Pharmaceuticals) ophthalmic drops comprising dexamethasone were recently approved for DED treatment. A specific issue with currently available eye drop formulations comprising cyclosporine and lifitegrast are tolerability issues such as burning and stinging, which can last from many weeks to even months. Furthermore, a relatively slow onset of action is generally observed using ophthalmic drops. In addition, ophthalmic drops may have to be administered several times per day as a large portion of the active ingredient is washed out quickly out of the eye and therefore exposure of the eye surface to the active agent may be short. For this reason, formulations often maximize concentration to compensate for this inefficiency, which may be associated with acute high concentrations on the ocular surface that may result in safety issues. In addition, burning, itching and stinging associated with preservatives, such as anti-microbial preservatives, included in ophthalmic drops may be observed. Further, as patients may need to administer the drops multiple times per day, daily life is highly affected and patient compliance may be low. As the administration of drops into the eye can be perceived as difficult, accuracy of drop delivery to the ocular surface may also be limited. Over- or underdosing may thus occur.

Glucocorticoids have been used to treat dry eye disease. However, glucocorticoids in ophthalmic drops when used long-term may lead to elevated intraocular pressure (IOP) and may induce cataract.

In view of the drawbacks and challenges experienced with current available treatments, novel treatment methods, which effectively deliver glucocorticoids in an appropriate dose and are effective over an extended period to treat DED, including episodic flares of DED of one or more weeks, while avoiding the need for daily glucocorticoid administrations would provide benefits for patients.

All references cited herein are hereby incorporated by reference for all purposes.

### OBJECTS AND SUMMARY OF THE INVENTION

It is an object of certain embodiments of the present invention to provide an ocular insert comprising a glucocorticoid such as dexamethasone that is effective for treating DED, in particular for acute treatment of DED, for instance, upon episodic flares in a patient during a period of one or more weeks.

Another object of certain embodiments of the present invention is to provide an ocular insert comprising a glucocorticoid such as dexamethasone that provides for sustained release of the glucocorticoid to the ocular surface through the tear fluid.

Another object of certain embodiments of the present invention is to provide an ocular insert comprising a glucocorticoid such as dexamethasone that provides for sustained release of the glucocorticoid to the ocular surface through the tear fluid, wherein the period of sustained release comprises a period of constant glucocorticoid release per day.

Another object of certain embodiments of the present invention is to provide an ocular insert comprising a glucocorticoid such as dexamethasone that provides for a treatment of DED for a period of one or more weeks, with only a single administration.

Another object of certain embodiments of the present invention is to provide an ocular insert comprising a glucocorticoid such as dexamethasone that increases tear retention in the eye.

Another object of certain embodiments of the present invention is to provide an ocular insert comprising a glucocorticoid such as dexamethasone that provides for sustained delivery of a glucocorticoid such as dexamethasone for both anti-inflammatory therapy and increased tear retention resulting in an additive or synergistic, beneficial effect that has both a rapid onset and that is maintained for an extended period of time after administration, such as a period of one or more weeks, with only a single administration.

Another object of certain embodiments of the present invention is to provide an ocular insert comprising a glucocorticoid such as dexamethasone that is sufficiently biodegradable, thereby avoiding the need for removal of the drug-depleted insert.

Another object of certain embodiments of the present invention is to provide an ocular insert comprising a glucocorticoid such as dexamethasone that, after the period of glucocorticoid release to the ocular surface through the tear film, has a prolonged therapeutic effect of tear retention.

Another object of certain embodiments of the present invention is to provide an ocular insert comprising a glucocorticoid such as dexamethasone that is biocompatible and low or non-immunogenic due to certain embodiments of the insert being free of animal- or human-derived components.

Another object of certain embodiments of the present invention is to provide an ocular insert comprising a glucocorticoid such as dexamethasone that is free of anti-microbial preservatives.

Another object of certain embodiments of the present invention is to provide an ocular insert comprising a glucocorticoid such as dexamethasone that is dimensionally stable when in a dry state but changes its dimensions upon hydration, e.g. after administration to the eye.

Another object of certain embodiments of the present invention is to provide an ocular insert comprising a glucocorticoid such as dexamethasone that is administered in a dry state and hydrates when inserted e.g. into the canaliculus.

Another object of certain embodiments of the present invention is to provide an ocular insert comprising a glucocorticoid such as dexamethasone that in its dry state is easy to administer but that is firmly secured in the canaliculus, avoiding potential insert loss during the treatment period, thereby providing improved retention, especially when compared to commonly applied plugs such as collagen or silicone plugs.

Another object of certain embodiments of the present invention is to provide an ocular insert comprising a glucocorticoid such as dexamethasone, wherein the insert is stable and has a defined shape and surface area both prior to as well as after insertion (i.e., inside the canaliculus).

Another object of certain embodiments of the present invention is to provide an ocular insert comprising a glucocorticoid such as dexamethasone that is easy to handle, in particular that does not spill or fragment easily.

Another object of certain embodiments of the present invention is to provide an ocular insert comprising a glucocorticoid such as dexamethasone that enables administration of an exact dose (within a broad dose range), thereby avoiding the risk of over- and under-dosing.

Another object of certain embodiments of the present invention is to provide an ocular insert comprising a glucocorticoid such as dexamethasone that does not cause glucocorticoid peaks or substantial peaks that could potentially result in adverse effects such as elevated intraocular pressure, glaucoma and cataract.

Another object of certain embodiments of the present invention is to provide an ocular insert comprising a glucocorticoid such as dexamethasone for the treatment of dry eye disease such as the acute treatment of DED that provides a lower incidence of side effects, such as burning, stinging or itching, as compared to commonly known dry eye therapies.

Another object of certain embodiments of the present invention is to provide an ocular insert comprising a glucocorticoid such as dexamethasone that provides a hands-free alternative for the patient compared to conventional DED treatments.

Another object of certain embodiments of the present invention is to provide an ocular insert comprising a glucocorticoid such as dexamethasone that is not subject to abuse, e.g. because it is administered by a physician.

Another object of certain embodiments of the present invention is to provide an ocular insert comprising a glucocorticoid such as dexamethasone that generally stays in the area of the eye to which it was administered, such as in the inferior and/or superior (vertical) canaliculus.

Another object of certain embodiments of the present invention is to provide an ocular insert comprising a glucocorticoid such as dexamethasone that is safe and well-tolerated.

Another object of certain embodiments of the present invention is to provide an ocular insert comprising a glucocorticoid such as dexamethasone that has increased patient compliance as compared to currently available DED treatments.

Another object of certain embodiments of the present invention is to provide an ocular insert comprising a glucocorticoid such as dexamethasone that can be visualized in a fast and simple manner and by a non-invasive method.

Another object of certain embodiments of the present invention is to provide an ocular insert comprising a glucocorticoid such as dexamethasone that provides for sustained release of a therapeutically effective amount of the glucocorticoid such as dexamethasone over an extended period of time, such as over a period of up to about 7 days, up to about 14 days, or up to about 21 days, or up to about 25 days after administration.

Another object of certain embodiments of the present invention is to provide an ocular insert comprising a glucocorticoid such as dexamethasone that releases a constant or essentially constant amount of the glucocorticoid such as dexamethasone over an extended period of time, such as for a period of up to about 7 days, or up to about 14 days, or up to about 21 days, or up to about 25 days after administration.

Another object of certain embodiments of the present invention is to provide an ocular insert comprising a glucocorticoid such as dexamethasone that provides for sustained release of a therapeutically effective amount of the glucocorticoid such as dexamethasone over an extended period of time after administration, such as over a period of up to about 7 days, up to about 14 days, or up to about 21 days, or up to about 25 days, thereby avoiding the need for frequent glucocorticoid administrations, which are required e.g. several times a day when using ophthalmic drops.

Another object of certain embodiments of the present invention is to provide an ocular insert comprising a glucocorticoid such as dexamethasone that provides for sustained release of a therapeutically effective amount of the glucocorticoid such as dexamethasone over an extended period of time after administration, such as for a period of up to about 7 days, up to about 14 days, or up to about 21 days, or up to about 25 days, wherein the glucocorticoid amount in the tear film is consistently maintained at a therapeutically effective level sufficient for anti-inflammatory therapy of the ocular surface.

Another object of certain embodiments of the present invention is to provide an ocular insert comprising a glucocorticoid such as dexamethasone that provides for sustained release of a therapeutically effective amount of the glucocorticoid such as dexamethasone over an extended period of time after administration, such as for a period of up to about 7 days, up to about 14 days, or up to about 21 days, or up to about 25 days, wherein essentially no toxic concentrations of the glucocorticoid are observed on the ocular surface and/or in the tear film.

Another object of certain embodiments of the present invention is to provide an ocular insert comprising a glucocorticoid such as dexamethasone that provides for sustained release of a therapeutically effective amount of the glucocorticoid such as dexamethasone over an extended period of time after administration, such as for a period of up to about 7 days, up to about 14 days, or up to about 21 days, or up to about 25 days, wherein the glucocorticoid is not resorbed systemically or not substantially resorbed systemically thereby minimizing or avoiding systemic toxicity.

Another object of certain embodiments of the present invention is to provide a method of treating DED, in particular of acute treatment (of episodic flares) of DED, in a patient in need thereof.

Another object of certain embodiments of the present invention is to provide a method of manufacturing an ocular insert comprising a glucocorticoid such as dexamethasone.

One or more of these objects of the present invention and others are solved by one or more embodiments of the invention as disclosed and claimed herein.

In certain embodiments, the present invention enables the effective short-term treatment of signs and symptoms of DED through topical glucocorticoids such as dexamethasone by means of a simple, hands-free (e.g., physician-administered) insert that combines the benefits of an anti-inflammatory glucocorticoid therapy with the effect of tear conservation by means of punctal occlusion into one single therapy. As the insert of the invention in certain embodiments is administered only once at the beginning of the treatment period (e.g., to treat episodic flares of DED), and no repeated administration by the patient is thus necessary, over- or underdosing can be avoided, as well as potential abuse/misuse. In certain embodiments, the dose of glucocorticoid such as dexamethasone and the duration of release of the active is adjusted to conform to a short treatment period, e.g., of about 2 or about 3 weeks as required for the treatment of episodic flares of DED. Exposure to unnecessarily high doses of glucocorticoid as well as unnecessarily long exposure to glucocorticoid are reduced, thereby avoiding as much as possible potential adverse side effects of glucocorticoids that may be associated with long-term and/or high dose use or dose peaks, and patient abuse or misuse. Also, in certain embodiments the inserts of the present invention do not require antimicrobial preservatives and thereby further reduce the potential of e.g. allergic reactions or adverse effects that can be associated with preservatives often used in topically applied ocular formulations. The inserts of the present invention may also increase patient compliance due to increased convenience, and increase the effectiveness of the treatment of DED, including episodic flares of DED.

The individual aspects of the present invention are disclosed in the specification and claimed in the independent claims, while the dependent claims claim particular embodiments and variations of these aspects of the invention. Details of the various aspects of the present invention are provided in the detailed description below.

### BRIEF DESCRIPTION OF THE DRAWINGS

FIG. 1 Schematic representation of an exemplary insert packaging. The insert is placed into a foam carrier and sealed with a foil pouch.

FIG. 2 Schematic exemplary representation of insert placement into the inferior vertical canaliculus through the lower punctum of the eye (A). Visualization of the insert is possible e.g. by illumination with blue light (B). The fluorescein in the intracanalicular insert in one embodiment illuminates when excited with blue light enabling confirmation of insert presence in a non-invasive manner.

FIG. 3 Schematic exemplary representation of insert dimensional change upon contact with tear fluid after insertion of the dry insert into the canaliculus where it is hydrated by the tear fluid.

FIG. 4 In vitro dexamethasone release from intracanalicular inserts according to embodiments of the invention comprising 0.2 mg and 0.3 mg dexamethasone over time (in PBS at a pH of 7.4 at 37° C.).

FIG. 5 Pharmacokinetic profile of dexamethasone release into tear fluid of beagle dogs from a 0.22 mg dexamethasone insert to illustrate release from an insert according to an embodiment of the invention. Inserts were placed bilaterally into the punctum of 7 beagles (i.e., a total of 14 eyes) on day 0. Tear fluid samples were collected from beagle eyes with 10 mm Schirmer tear test strips on days 1, 2, 4, 7, 10, 14, 17, 21, 28, 35, 37, and 40 after insertion of the insert into the canaliculus. Dexamethasone levels in tear fluid were measured by LC-MS/MS. Dexamethasone is presented as average values together with corresponding standard deviation error bars. Number of samples measured: For day 1, n was 6 eyes; for day 2, n was 8 eyes; for days 14 and 21, n was 7 eyes; for day 28, n was 6 eyes; for day 35, n was 2 eyes. A single insert delivered dexamethasone to the ocular surface for approximately 14 days, with a sustained level of dexamethasone in the tear fluid maintained through day 7, followed by a tapering from day 7 to day 14 with complete release by day 17.

FIG. 6 Dexamethasone release from a 0.37 mg dexamethasone insert to illustrate release from an insert according to an embodiment of the invention at different study time-points. Dexamethasone is released over time into the tear fluid primarily from the insert site proximal to the punctum opening. The darker shading of the insert reflects the presence of dexamethasone, and the clearing reflects the zone of the insert depleted of dexamethasone. Dexamethasone is essentially completely released from the 0.37 mg insert after 28 days.

FIG. 7 outlines a Phase 2 Clinical Study of Example 4. After a 14-day wash-out period patients are administered either the 0.2 mg dexamethasone insert, the 0.3 mg dexamethasone insert, or the hydrogel vehicle, i.e. the insert without dexamethasone.

FIG. 8 depicts demographics and baseline measurements for the clinical trial of Example 4. There were 52 screen failures of 224 screened.

FIG. 9 depicts the retention rate by Insert Presence Visualization from the study of Example 4.

FIGS. 10A-B depicts statistically significant improvement in primary endpoint (conjunctival hyperemia in the worst zone) for an inventive insert relative to vehicle hydrogel for 0.2 and 0.3 mg from the study of Example 4.

FIGS. 11A-B depicts the conjunctival hyperemia endpoint from the study of Example 4

FIGS. 12A-F depicts the conjunctival hyperemia nasal, temporal and frontal endpoint from the study of Example 4.

FIGS. 13A-B depicts the eye dryness severity symptoms scores (VAS) improved from baseline in 0.2 and 0.3 mg groups from the study of Example 4.

FIG. 14 depicts treatment emergent adverse events from the study of Example 4.

FIG. 15 depicts most common adverse events in treated groups—epiphora (lacrimation increase (8.1%), IOP elevation (3.6%) from the study of Example 4.

FIG. 16 depicts systemic adverse events from the study of Example 4.

FIGS. 17A-B depict the CCLRU grading scale (A) and regions for grading (B) for photographic assessment of bulbar conjunctival hyperemia change from baseline (CFB) (evaluated via central reading center, CCLRU grading scale, 0-4 per region)—worst zone (primary endpoint) and bulbar conjunctival hyperemia using CCLRU grading score,

CFB, individual zones and total (secondary endpoint).

FIG. 18 is the scale for Eye Dryness Score (visual analogue scale (VAS)), CFB and absolute values at each post baseline study visit.

### DEFINITIONS

The term “insert” as used herein refers to an object that contains an active agent, specifically a glucocorticoid such as dexamethasone and that is administered into the human or animal body, such as to the canaliculus of the eye (one eye or both eyes, as well as inferior and/or superior canaliculus), where it remains for a certain period of time while it releases the active agent into the surrounding environment. An insert can have any predetermined shape before being inserted, which general shape may be maintained to a certain degree upon placing the insert into the desired location, although dimensions of the insert (e.g. length and/or diameter) may change after administration due to hydration as further disclosed herein. In other words, what is administered into the canaliculus of the eye is not a solution or suspension, but an already shaped, coherent object. The insert has thus been completely formed, e.g., according to the methods disclosed herein prior to being administered. Over the course of time the inserted insert in certain embodiments is biodegraded (as disclosed herein), and may thereby change its shape (e.g. may expand in diameter and decrease in length) until it has been completely dissolved/resorbed. Herein, the term “insert” is used to refer both to an insert in a hydrated (also referred to herein as “wet”) state when it contains water, e.g. after the insert has been hydrated or re-hydrated once administered to the eye or otherwise immersed into an aqueous environment (such as in vitro), as well as to an insert in its/a dry (dried/dehydrated) state. Thus, in certain embodiments, an insert in its dry/dried state in the context of the present invention may contain no more than about 1% by weight water. The water content of an insert in its dry/dried state may be measured e.g. by means of a Karl Fischer coulometric method. Whenever dimensions of an insert (i.e., length, diameter, or volume) are reported herein in the hydrated state, these dimensions are measured after the insert has been immersed in phosphate-buffered saline at a pH value of 7.4 at 37° C. for 24 hours. Whenever dimensions of an insert are reported herein in the dry state, these dimensions are measured after the insert has been fully dried (and thus, in certain embodiments, contains no more than about 1% by weight water). In certain embodiments, the insert is kept in an inert atmosphere glove box containing below 20 ppm of both oxygen and moisture for at least about 7 days.

In certain embodiments of the present invention, the term “fiber” (used interchangeably herein with the term “rod”) characterizes an object (i.e., in the present case an insert according to certain embodiments of the present invention) that in general has an elongated shape. Specific dimensions of inserts of the present invention are disclosed herein. The insert may have a cylindrical or essentially cylindrical shape, or may have a non-cylindrical shape. The cross-sectional area of the fiber or the insert may be either round or essentially round, but may in certain embodiments also be oval or oblong, or may in other embodiments have different geometries, such as cross-shaped, star-shaped or other as disclosed herein.

The term “ocular” as used herein refers to the eye in general, or any part or portion of the eye (as an “ocular insert” according to the invention refers to an insert that can in principle be administered to any part or portion of the eye). The present invention in certain embodiments is directed to intracanalicular administration of an ocular insert (in this case the “ocular insert” is thus an “intracanalicular insert”), and to the treatment of dry eye disease (DED), as further disclosed herein.

The term “biodegradable” as used herein refers to a material or object (such as the intracanalicular insert according to the present invention) which becomes degraded in vivo, i.e., when placed in the human or animal body. In the context of the present invention, as disclosed in detail herein, the insert comprising the hydrogel within which particles of a glucocorticoid, such as particles of dexamethasone, are dispersed, slowly biodegrades over time once deposited within the eye, e.g., within the canaliculus. In certain embodiments, biodegradation takes place at least in part via ester hydrolysis in the aqueous environment provided by the tear fluid. In certain embodiments, the intracanalicular inserts of the present invention slowly soften and liquefy, and are eventually cleared (disposed/washed out) through the nasolacrimal duct.

A “hydrogel” is a three-dimensional network of one or more hydrophilic natural or synthetic polymers (as disclosed herein) that can swell in water and hold an amount of water while maintaining or substantially maintaining its structure, e.g., due to chemical or physical cross-linking of individual polymer chains. Due to their high water content, hydrogels are soft and flexible, which makes them very similar to natural tissue. In the present invention the term “hydrogel” is used to refer both to a hydrogel in the hydrated state (also referred to herein synonymously as the “wet state”) when it contains water (e.g. after the hydrogel has been formed in an aqueous solution, or after the hydrogel has been hydrated or re-hydrated once inserted into the eye or otherwise immersed into an aqueous environment) and to a hydrogel in its/a dry (dried/dehydrated) state when it has been dried to a low water content of e.g. not more than 1% by weight as disclosed herein. In the present invention, wherein an active principle is contained (e.g. dispersed) in a hydrogel, the hydrogel may also be referred to as a “matrix”.

The term “polymer network” as used herein describes a structure formed of polymer chains (of the same or different molecular structure and of the same or different average molecular weight) that are cross-linked with each other. Types of polymers suitable for the purposes of the present invention are disclosed herein. The polymer network may be formed with the aid of a crosslinking agent as also disclosed herein.

The term “amorphous” refers to a polymer or polymer network or other chemical substance or entity which does not exhibit crystalline structures in X-ray or electron scattering experiments.

The term “semi-crystalline” refers to a polymer or polymer network or other chemical substance or entity which possesses some crystalline character, i.e., exhibits some crystalline properties in X-ray or electron scattering experiments.

The term “crystalline” refers to a polymer or polymer network or other chemical substance or entity which has crystalline character as evidenced by X-ray or electron scattering experiments. The term “precursor” or “polymer precursor” or specifically “PEG precursor” herein refers to those molecules or compounds that are reacted with each other and that are thus connected via crosslinks to form a polymer network and thus the hydrogel matrix. While other materials might be present in the hydrogel, such as active agents, visualization agents or buffers, they are not referred to as “precursors”.

The molecular weight of a polymer precursor as used for the purposes of the present invention and as disclosed herein may be determined by analytical methods known in the art. The molecular weight of polyethylene glycol can for example be determined by any method known in the art, including gel electrophoresis such as SDS-PAGE (sodium dodecyl sulphate-polyacrylamide gel electrophoresis), gel permeation chromatography (GPC), including GPC with dynamic light scattering (DLS), liquid chromatography (LC), as well as mass spectrometry such as matrix-assisted laser desorption/ionization-time of flight (MALDI-TOF) spectrometry or electrospray ionization (ESI) mass spectrometry. The molecular weight of a polymer, including a polyethylene glycol precursor as disclosed herein, is an average molecular weight (based on the polymer's molecular weight distribution), and may therefore be indicated by means of various average values, including the weight average molecular weight (Mw) and the number average molecular weight (Mn). Any of such average values may generally be used in the context of the present invention. In the context of the present invention, the average molecular weight of the polyethylene glycol units or other precursors or units as disclosed herein is the number average molecular weight (Mn) and is indicated in the unit “Daltons”.

The parts of the precursor molecules that are still present in a final polymer network are also called “units” herein. The “units” are thus the building blocks or constituents of a polymer network forming the hydrogel. For example, a polymer network suitable for use in the present invention may contain identical or different polyethylene glycol units as further disclosed herein.

As used herein, the term “crosslinking agent” or “crosslinker” refers to any molecule that is suitable for connecting precursors via crosslinks to form the polymer network and thus the hydrogel matrix. In certain embodiments, crosslinking agents may be low-molecular weight compounds or may be polymeric compounds as disclosed herein.

The term “sustained release” is generally defined for the purposes of the present invention to refer to pharmaceutical dosage forms or products (in the case of the present invention these products are inserts) which are formulated to make an active, such as a glucocorticoid according to the present invention, specifically including but not limited to dexamethasone, available over an extended period of time after administration, such as one or more weeks, thereby allowing a reduction in dosing frequency compared to an immediate release dosage form, e.g. a solution of a glucocorticoid that is topically applied onto the eye (i.e. glucocorticoid-comprising eye drops). Other terms that may be used herein interchangeably with “sustained release” are “extended release” or “controlled release”. “Sustained release” thus generally characterizes the release of an API, specifically, the glucocorticoid, such as dexamethasone, that is contained in an insert according to the present invention. The term “sustained release” per se is not associated with or limited to a particular rate of (in vitro or in vivo) release, although in certain embodiments of the invention an insert may be characterized by a certain average rate of (in vitro or in vivo) release or a certain release profile as disclosed herein. As an insert of the present invention (whether explicitly referred to herein as a “sustained release” insert or simply as an “insert”) provides for sustained release of the API, an insert of the present invention may therefore also be referred to as a “depot”.

Within the specific meaning of the present invention, the term “sustained release” also comprises a period of constant or substantially constant (i.e, above a certain level) glucocorticoid release per day when this period of constant or substantially constant release is followed by a period of tapered glucocorticoid release. In such specific case, an overall sustained release provided by an insert of the present invention (as defined above) may mean that the release rate is not necessarily constant or essentially constant throughout the entire period of glucocorticoid release, but may change over time as just described (i.e., with an initial period of constant or essentially constant, i.e., sustained release, followed by a period of tapered release). Within the meaning of the invention, the term “tapered” or “tapering” refers to a decreasing release of glucocorticoid such as dexamethasone over time until the glucocorticoid is completely released.

The term “visualization agent” as used herein refers to a molecule or composition that may be contained within an insert of the present invention and that provides the possibility of easily visualizing the insert in a non-invasive manner when it is located in the canaliculus of the eye, e.g. by illuminating the corresponding eye parts with a suitable light source, such as blue light. The visualization agent may be a fluorophore such as fluorescein, rhodamine, coumarin, and cyanine, or other suitable agents as disclosed herein. In certain embodiments the visualization agent is fluorescein or includes a fluorescein moiety.

As used herein, the term “ocular surface” comprises the conjunctiva and the cornea, together with elements such as the lacrimal apparatus, including the lacrimal punctum, as well as the lacrimal canaliculus and associated eyelid structures. Within the meaning of this invention, the ocular surface encompasses also the aqueous humor.

As used herein, the terms “tear fluid” or “tears” or “tear film” refer to the liquid secreted by the lacrimal glands, which lubricates the eyes. Tears are made up of water, electrolytes, proteins, lipids, and mucins.

As used herein, the term “bilaterally” or “bilateral” refers (in the context of administration of the inserts of the present invention) to an administration of the inserts into both eyes of a patient. “Unilaterally” or “unilateral” thus refers to an administration of the insert into one eye only. The inserts may be independently inserted into the superior and/or the inferior canaliculus of both eyes or of one eye.

As used herein, the terms “administration” or “administering” or “administered” etc. in the context of the inserts of the present invention refer to the process of insertion of the inserts through the opening of the punctum into the canaliculus of the eye. Thus, “administering an insert” or similar terms refer to the insertion of the insert into the canaliculus. The terms “insertion” or “inserting” or “inserted” etc. in the context of the inserts of the present invention equally refer to the process of insertion of the inserts through the opening of the punctum into the canaliculus of the eye and are thus herein used interchangeably with the terms “administration” or “administering” or “administered”. In contrast, the terms “administration” or “administering” or “administered” etc. in the context of topical ophthalmic pharmacological products such as eye drops (which are not the subject of the present invention) refer to topical application of these products onto the eye.

As used herein, the term “insert stacking” or “stacking” refers to the insertion of a further insert on top of a first insert while the first insert is still retained in the canaliculus (because it has not yet sufficiently biodegraded and/or has not yet cleared through the nasolacrimal duct). In certain embodiments, the further insert is placed on top of the first insert after the glucocorticoid contained in the first insert is completely or essentially completely released, or after at least about 70% or at least about 80% or at least about 90% of the glucocorticoid contained in the first insert has been released. Insert stacking enables, for instance, prolonged glucocorticoid treatment.

The term “plug” as used herein refers to a device capable of providing an occlusion, substantial occlusion or partial occlusion of the tear duct(s) (“lacrimal occlusion”) thereby minimizing or preventing draining of tears. A plug thus increases tear retention, which helps to keep the eyes moist. Plugs can be classified into “punctal plugs” and “intracanalicular plugs”. Intracanalicular plugs are also referred to as “canalicular plugs” in literature. Both plug classes are inserted through the upper and/or lower punctum of the eye. Punctal plugs rest at the punctal opening making them easily visible and, hence, removable without much difficulty. However, punctal plugs may show poor retention rates and can be more easily contaminated with microbes due to their exposed localization which may result in infection. In contrast, intracanalicular plugs are essentially not visible and provide a better retention rate compared to punctal plugs as they are placed inside either the vertical or the horizontal canaliculus. However, currently available intracanalicular plugs may not be easy to remove and/or may provide an increased risk of migration due to loose fit. Commercially available plugs are often made of collagen, acrylic polymers, or silicone.

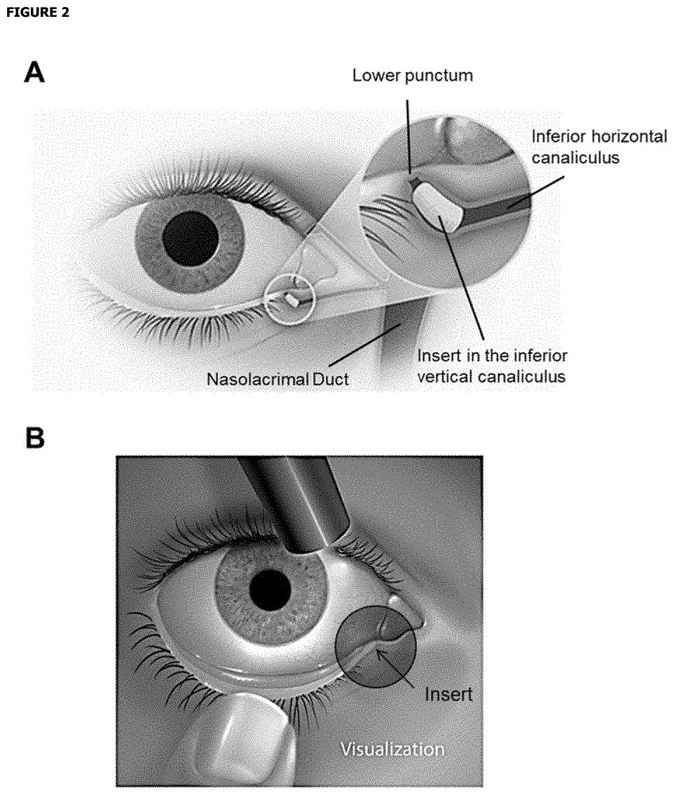

The terms “canaliculus” (plural “canaliculi”) or alternatively “tear duct” as used herein refer to the lacrimal canaliculus, i.e. the small channels in each eyelid that drain lacrimal fluid (tear fluid) from the lacrimal punctum to the nasolacrimal duct (see also FIG. 2A). Canaliculi therefore form part of the lacrimal apparatus that drains lacrimal fluid from the ocular surface to the nasal cavity. The canaliculus in the upper eyelid is referred to as “superior canaliculus” or “upper canaliculus”, whereas the canaliculus in the lower eyelid is referred to as “inferior canaliculus” or “lower canaliculus”. Each canaliculus comprises a vertical region, referred to as “vertical canaliculus” following the lacrimal punctum and a horizontal region, referred to as “horizontal canaliculus” following the vertical canaliculus, wherein the horizontal canaliculus merges into the nasolacrimal duct.

The term “punctum” (plural “puncta”) refers to the lacrimal punctum, an opening on the margins of the eyelids, representing the entrance to the canaliculus. After tears are produced, some fluid evaporates between blinks, and some is drained through the lacrimal punctum. As both the upper and the lower eyelids show the lacrimal punctum, the puncta are therefore referred to as “upper punctum” or “superior punctum” and “lower punctum” or “inferior punctum”, respectively (see also FIG. 2A).

The term “intracanalicular insert” refers to an insert that can be administered through the upper and/or lower punctum into the superior and/or inferior canaliculus of the eye, in particular into the superior and/or inferior vertical canaliculus of the eye. Due to the intracanalicular localization of the insert, the insert blocks tear drainage through lacrimal occlusion such as also observed for intracanalicular plugs. The intracanalicular inserts of the present invention may be inserted bilaterally or unilaterally into the inferior and/or superior vertical canaliculi of the eyes. According to certain embodiments of the present invention, the intracanalicular insert is a sustained release biodegradable insert.

The terms “API”, “active (pharmaceutical) ingredient”, “active (pharmaceutical) agent”, “active (pharmaceutical) principle”, “(active) therapeutic agent”, “active”, and “drug” are used interchangeably herein and refer to the substance used in a finished pharmaceutical product (FPP) as well as the substance used in the preparation of such a finished pharmaceutical product, intended to furnish pharmacological activity or to otherwise have direct effect in the diagnosis, cure, mitigation, treatment or prevention of a disease, or to have direct effect in restoring, correcting or modifying physiological functions in a patient.

The API used according to the present invention is a glucocorticoid such as dexamethasone. Glucocorticoids are a class of corticosteroids, which are a class of steroid hormones. The name “glucocorticoid” is a portmanteau (glucose+cortex+steroid) and is composed from its role in regulation of glucose metabolism, synthesis in the adrenal cortex, and its steroidal structure. A less common synonym is glucocorticosteroid. Glucocorticoids act through glucocorticoid receptor-mediated pathways present in most cells in the body to regulate gene expression, and through non-receptor pathways to inhibit inflammatory cytokine (TNF alpha, IL-1a, and IL-6) and chemokine production and decrease the synthesis of matrix metalloproteinases (Rosenbaum et al., 1980; Nature 286(5773): 611-613). Glucocorticoids, such as dexamethasone, suppress inflammation by inhibiting edema, fibrin deposition, capillary deposition, and phagocytic migration of the inflammatory response (Chrousos 1995, NEJM 332(20): 1351-1362; Abelson et al. 2002, Review of Ophthalmology: 110-114; Sherif and Pleyer 2002, Ophthalmologica 216(5): 305-315). As in other tissues, glucocorticoids do not appear to have a specific mechanism of action in ocular tissues but exert a broad spectrum of anti-inflammatory activity (Leopold 1985, M. L. Sears and A. Tarkkanen, ed. New York, Raven Press: 83-133; Kaiya 1990, J Cataract Refract Surg 16 (3): 320-324). In general, most uses of glucocorticoids are limited to a relatively short duration (about 2 to 3 weeks), due to concerns regarding potential side effects associated with prolonged use. Cortisol (or the synthetic form, referred to as hydrocortisone) is the most important human glucocorticoid. In addition, a variety of synthetic glucocorticoids with varying potencies has been created for therapeutic use. Examples of synthetic glucocorticoids are prednisone, prednisolone, prednisolone acetate, methylprednisolone, dexamethasone, dexamethasone acetate, betamethasone, betamethasone sodium phosphate, budesonide, flunisolide, fluticasone propionate, triamcinolone, triamcinolone acetonide, triamcinolone hexacetonide, triamcinolone diacetate, fluocinolone acetonide, fludrocortisone acetate, loteprednol, loteprednol etabonate, difluprednate, fluorometholone, mometasone furoate, deoxycorticosterone acetate, aldosterone, rimexolone, beclometasone, and beclomethasone dipropionate. Any of these synthetic glucocorticoids are suitable for use in the present invention. In particular embodiments of the invention the glucocorticoid is a low solubility glucocorticoid (i.e., having a solubility in water of less than about 100 μg/mL), including (but not limited to) beclomethasone dipropionate, betamethasone sodium phosphate, budesonide, flunisolide, fluticasone propionate, triamcinolone acetonide, triamcinolone hexacetonide, triamcinolone diacetate, dexamethasone, dexamethasone acetate, prednisolone acetate, loteprednol etabonate, difluprednate, fluorometholone, fluocinolone acetonide, and mometasone furoate. Dexamethasone is sometimes also referred to as “dexamethasone alcohol”.

In general, glucocorticoid potencies are reported as relative potencies in view of cortisol potency. Determination of equivalent glucocorticoid doses is well established in the art. Equivalent oral doses and relative oral glucocorticoid potencies are presented in Table 1 for exemplarily selected glucocorticoids (see for instance, Buttgereit et al. 2002, Ann Rheum Dis 61:718-722, which is incorporated herein by reference).

TABLE 1
Established equivalent oral doses and relative oral glucocorticoid potencies
(with reference to cortisol) of exemplarily selected glucocorticoids.

| | | Equiv- | | | Equiv- |
|---|---|---|---|---|---|
| | Relative | alent | | Relative | alent |
| Glucocorticoid | Potency | Dose | Glucocorticoid | Potency | Dose |
| Cortisol | 1 | 20 mg | Dexamethasone | 25-80 | 0.8 mg |
| (Hydrocortisone) | | | Betamethasone | 25-30 | 0.8 mg |
| Prednisone | 3.5-5 | 5 mg | Triamcinolone | 5 | 4 mg |
| Prednisolone | 4 | 5 mg | | | |
| Methyl- | 5-7.5 | 4 mg | | | |
| prednisolone | | | | | |

As used herein, the term “equivalent dose” refers to a dose of an active such as a glucocorticoid that is equivalent in terms of biological activity to a dose of another active such as another glucocorticoid when delivered via the same administration route (e.g. oral, intravenous, topical, or via the intracanalicular inserts of the present application). Examples for equivalent oral doses of glucocorticoids are presented in Table 1. For instance, upon oral administration of 20 mg hydrocortisone, similar biological effects are to be expected when compared to oral administration of 0.8 mg dexamethasone.

In certain embodiments, the glucocorticoid used according to the present invention is dexamethasone. Dexamethasone is a long-acting anti-inflammatory 9-fluoro glucocorticoid (also termed a glucocorticoid agonist) with a molecular weight of 392.47 g/mol. The molecular formula of dexamethasone is C₂₂H₂₉FO₅ and its IUPAC name is 9-Fluor-11β,17,21-trihydroxy-16α-methyl-pregna-1,4-dien-3,20-dion (CAS No. 50-02-2). The chemical structure of dexamethasone is reproduced below:

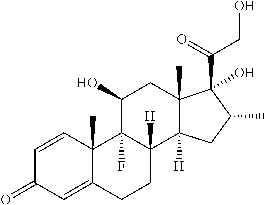

Dexamethasone is a white to practically white, odorless crystalline powder with poor solubility in water (approx. 89 mg/L at 25° C.). Its partition coefficient (n-octanol/water) is 1.83 (log P; cf. DrugBank entry “dexamethasone”).

In certain embodiments, for any glucocorticoid used in the present invention, including dexamethasone, particle sizes (e.g. as expressed by the d90 value) of about 100 μm or below, or of about 75 μm or below, or of about 50 μm or below may be used. In particular embodiments of the present invention, dexamethasone may be used in the form of micronized particles and may have a d90 particle size of equal to or less than about 100 μm, or of equal to or less than about 75 μm, or of equal to or less than about 50 μm, or of equal to or less than about 20 μm, or of equal to or less than about 10 μm, or of equal to or less than about 5 μm. In these and other embodiments, the d98 particle size of the micronized dexamethasone may be equal to or less than about 100 μm, or equal to or less than about 75 μm, or equal to or less than about 50 μm, or equal to or less than about 20 μm, or equal to or less than about 10 μm, or equal to or less than about 5 μm. In particular embodiments of the present invention, the micronized dexamethasone has a d90 particle size of equal to or less than about 5 μm and a d98 particle size of less than about 10 μm. The “d90” value means that at least 90 volume-% of all particles within the measured bulk material (which has a certain particle size distribution) has a particle size below the indicated value. For example, a d90 particle size of less than about 50 μm means that at least 90 volume-% of the particles in the measured bulk material have a particle size below about 50 μm. Corresponding definitions apply to other “d” values, such as the “d98” value. The particle size distribution can be measured by methods known in the art, including sieving, laser diffraction or dynamic light scattering. In embodiments in which another glucocorticoid than dexamethasone is used in the present invention similar particle sizes may apply as disclosed for dexamethasone.

For the purposes of certain embodiments of the present invention, in order to increase content uniformity in the final product (and thus to avoid a too high or too low drug content due to discrete particles) and/or reduce potential agglomeration of discrete API particles during manufacturing of the insert (such as during casting the hydrogel as disclosed herein) it may be useful to ensure—in addition to fulfilling a certain particle size specification (e.g. d90 and/or d98 value as disclosed herein)—that there or no or essentially no discrete particles present in the API starting material that are larger than a certain size, such as larger than about 120 μm, or larger than about 100 μm, or larger than about 90 μm. This can be achieved for example by sieving. In particular embodiments, the dexamethasone used for manufacturing the inserts according to the present invention has a d90 particle size of equal to or less than about 5 μm, and a d98 particle size of less than about 10 μm, with all or essentially all discrete particles having a size of less than about 90 μm.

For the purposes of the present invention, active agents (including dexamethasone) in all their possible forms, including any active agent polymorphs or any pharmaceutically acceptable salts, anhydrates, hydrates, other solvates or derivatives of active agents, can be used. Whenever in this description or in the claims an active agent is referred to by name, e.g., “dexamethasone”, even if not explicitly stated, it also refers to any such pharmaceutically acceptable polymorphs, salts, anhydrates, solvates (including hydrates) or derivatives of the active agent. Particularly, the term “dexamethasone” refers to dexamethasone and pharmaceutically acceptable salts thereof, which may all be used for the purposes of the present invention. The term “polymorph” as used herein refers to any crystalline form of an active agent such as dexamethasone. Frequently, active agents that are solid at room temperature exist in a variety of different crystalline forms, i.e., polymorphs, with one polymorph being the thermodynamically most stable at a given temperature and pressure.

Any and all dexamethasone polymorphs (whether anhydrous forms or solvates) can be used for preparing inserts according to the embodiments of the present invention. With respect to dexamethasone, suitable solid forms including amorphous forms and polymorphs of dexamethasone (without being limited by any of these) are for example disclosed in Oliveira et al., Cryst. Growth Des. 2018, 18, 1748-1757 and in Aljarah et al., Indian Journal of Forensic Medicine & Toxicology, January-March 2019, Vol. 13, No. 1, 372-377 or in any references cited in any of these.

In addition to dexamethasone (alcohol) itself, suitable solid forms of dexamethasone for use in the present invention include for example (without being limited to these) dexamethasone sodium phosphate, dexamethasone acetate, dexamethasone benzoate, dexamethasone 21-(adamantine-1-carboxylate), dexamethasone isonicotinate, dexamethasone valerate, dexamethasone tebutate, dexamethasone 21-sulfobenzoate, dexamethasone sodium-metasulfobenzoate, dexamethasone palmitate, dexamethasone cipecilate, dexamethasone carboxamide, dexamethasone propionate as well as any mixtures thereof.

As used herein, the term “therapeutically effective” refers to the amount of drug or active agent (i.e. glucocorticoid) required to produce a desired therapeutic response or result after administration. For example, in the context of the present invention, one desired therapeutic result would be the reduction of symptoms associated with DED. The abbreviation “DED” as used herein refers to “dry eye disease” or “dry eye”.

As used herein, the terms “dry eye disease”, “DED”, or “dry eye” are used interchangeably and bear equivalent meanings. DED is also referred to as “keratoconjunctivitis sicca (KCS)” in literature. DED refers to a multifactorial disorder of the tears and the ocular surface characterized by symptoms of dryness, irritation, burning, stinging, grittiness, foreign body sensation, tearing, and ocular fatigue. Although the pathogenesis of DED is not fully understood, it is recognized that inflammation has a prominent role in the development and propagation of this debilitating condition. Factors that adversely affect tear film stability and osmolarity can induce ocular surface damage and initiate an inflammatory cascade that activates innate and adaptive immune responses. These immunoinflammatory responses lead to further ocular surface damage and the development of a self-perpetuating inflammatory cycle. DED is thought to be a chronic state, with episodic flares encompassing rapid onset of symptoms or symptom worsening, highly affecting daily life of patients.

As used herein, the term “episodic flares” refers to a condition, wherein rapid onset of DED symptoms or DED symptom worsening occurs. In between the relatively short-term episodic flares of DED, symptoms may mostly not be experienced or only mildly experienced. In certain embodiments, the present invention specifically relates to the acute (short-term) treatment of episodic flares of DED such as, for example, over a period of up to about 2 weeks or about 2 weeks, or up to about 3 weeks or about 3 weeks, or up to about 4 weeks or about 4 weeks.

As used herein, the terms “acute treatment” or “acute treating” or similar phrases refer to a short-term treatment or short-term treating of DED, such as of episodic flares of DED. In certain embodiments of the invention, the acute treatment period with a glucocorticoid contained in an insert according to the present invention is about one week or more weeks such as about 2 or about 3 weeks.

The term “patient” herein includes both human and animal patients. The inserts according to the present invention are generally suitable for human or veterinary medicinal applications. The patients enrolled and treated in a clinical study may also be referred to as “subjects”. Generally, a “subject” is a (human or animal) individual to which an insert according to the present invention is administered, such as during a clinical study. A “patient” is a subject in need of treatment due to a particular physiological or pathological condition.

The term “average” as used herein refers to a central or typical value in a set of data(points), which is calculated by dividing the sum of the data(points) in the set by their number (i.e., the mean value of a set of data).

As used herein, the term “about” in connection with a measured quantity refers to the normal variations in that measured quantity, as expected by one of ordinary skill in the art in making the measurement and exercising a level of care commensurate with the objective of measurement and the precision of the measuring equipment.

As used herein, the term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that.

As used herein, the singular forms “a,” “an”, and “the” include plural references unless the context clearly indicates otherwise.

The term “and/or” as used in a phrase such as “A and/or B” herein is intended to include both “A and B” and “A or B”.

Open terms such as “include,” “including,” “contain,” “containing” and the like as used herein mean “comprising” and are intended to refer to open-ended lists or enumerations of elements, method steps, or the like and are thus not intended to be limited to the recited elements, method steps or the like but are intended to also include additional, unrecited elements, method steps or the like.

The term “up to” when used herein together with a certain value or number is meant to include the respective value or number. For example, the term “up to 25 days” means “up to and including 25 days”.

The abbreviation “PBS” when used herein means phosphate-buffered saline.

The abbreviation “PEG” when used herein means polyethylene glycol.

All references disclosed herein are hereby incorporated by reference in their entireties for all purposes (with the instant specification prevailing in case of conflict).

### DETAILED DESCRIPTION

### I. The Insert

The present invention generally relates to a sustained release biodegradable ocular insert comprising a hydrogel and a glucocorticoid, wherein glucocorticoid particles are dispersed within the hydrogel. In certain embodiments, the insert is for administration into the canaliculus of the eye, i.e., is an intracanalicular insert.

The present invention in one aspect relates to a sustained release biodegradable ocular (such as intracanalicular) insert comprising a hydrogel and a glucocorticoid, wherein glucocorticoid particles are dispersed within the hydrogel, and wherein the insert in its dry state has a length of less than about 2.75 mm.

The present invention in another aspect generally relates to a sustained release biodegradable ocular (such as intracanalicular) insert comprising a hydrogel and equal to or less than about 375 μg dexamethasone or an equivalent dose of another glucocorticoid.

The present invention in another aspect generally relates to a sustained release biodegradable ocular (such as intracanalicular) insert comprising a hydrogel and a glucocorticoid, wherein the insert in a dry state (such as prior to being administered) has a length of equal to or less than about 2.75 mm.

The present invention in another aspect generally relates to a sustained release biodegradable ocular (such as intracanalicular) insert comprising a hydrogel and a glucocorticoid, wherein the insert provides for a release of a therapeutically effective amount of the glucocorticoid for a period of up to about 25 days after administration.

In all these aspects, a particular glucocorticoid for use in the present invention is dexamethasone.

In all these aspects, the ocular insert in certain embodiments of the invention may be an intracanalicular insert, i.e., the insert is for insertion/administration into the canaliculus of one or both eye(s).

Each of the features recited above in the three aspects of the invention may be present in a sustained release biodegradable insert of the invention separately, or any two of these features may be present in combination, or all three of these features may be present in combination.

Thus, in one specific embodiment the present invention relates to a sustained release biodegradable intracanalicular insert comprising a hydrogel and dexamethasone as the glucocorticoid, wherein the insert comprises equal to or less than about 375 μg dexamethasone, has a length of equal to or less than about 2.75 mm and provides for a release of a therapeutically effective amount of dexamethasone for a period of up to about 25 days after administration.

Specific embodiments and features of the insert of the present invention are disclosed below.

### The Active Principle

The present invention in certain embodiments generally relates to a sustained release biodegradable ocular (such as an intracanalicular) insert comprising a hydrogel and a glucocorticoid. One particular glucocorticoid for use in all aspects of the present invention is dexamethasone. Details on dexamethasone, its chemical structure and its properties such as solubility are disclosed herein in the definitions section.

In one embodiment, the present invention relates to a sustained release biodegradable intracanalicular insert comprising a hydrogel and equal to or less than about 375 μg dexamethasone or an equivalent dose of another glucocorticoid.

In another embodiment, the present invention relates to a sustained release biodegradable intracanalicular insert comprising a hydrogel and equal to or less than about 350 μg dexamethasone or an equivalent dose of another glucocorticoid.

In particular embodiments of the present invention the glucocorticoid contained in a sustained release biodegradable ocular (such as intracanalicular) insert is dexamethasone, and is present in the insert in a range of doses, from about 100 μg to about 350 μg, or from about 150 μg to about 320 μg. Any dexamethasone amount within these dose ranges may be used, such as about 100 μg, about 150 μg, about 200 μg, about 250 μg, about 300 μg, about 320 μg, about 350 μg etc., all values also including a variance of +25% and −20%, or a variance of ±10%. In certain particular embodiments, the doses of dexamethasone contained in an insert of the invention are:

In a range from about 160 μg to about 250 μg, or in a range from about 180 μg to about 220 μg, or in very particular embodiments about 200 μg (i.e., including a variance of +25% and −20%, or a variance of ±10% of 200 μg), or

In a range from about 240 μg to about 375 μg, or in a range from about 270 μg to about 330 μg, or in very particular embodiments about 300 μg (i.e., including a variance of +25% and −20%, or a variance of ±10% of 300 μg).

In alternative embodiments, the dose of the glucocorticoid such as dexamethasone in the insert of the invention may be from about 50 μg to about 500 μg, such as about 50 μg, about 100 μg, about 150 μg, about 200 μg, about 250 μg, about 300 μg, about 350 μg, about 400 μg, about 450 μg, or about 500 μg dexamethasone or an equivalent dose of another glucocorticoid. In exceptional cases, such dose may be above 500 μg.

If a glucocorticoid other than dexamethasone is used in a sustained release biodegradable intracanalicular insert according to the invention, a dose of that other glucocorticoid is contained in the insert that is equivalent to any of the dose amounts and ranges disclosed above for dexamethasone. Suitable conversion factors between glucocorticoids are known in the art and may be applied (see the section “Definitions” above).

The disclosed amounts of glucocorticoid, such as dexamethasone, including the mentioned variances, refer to both the final content of the active principle in the insert, as well as to the amount of active principle used as a starting component when manufacturing the insert.

In a specific embodiment, in case the sustained release biodegradable intracanalicular insert of the present invention is defined by a length of equal to or less than about 2.75 mm (as disclosed herein below) and/or a release of a therapeutically effective amount of a glucocorticoid for a period of up to about 25 days after administration (as disclosed herein below), the dose of the dexamethasone (or equivalent dose of another glucocorticoid) contained in the insert may also exceed 375 μg, and may also be about 400 μg or higher, such as from about 400 μg to about 600 μg, or of about 500 μg dexamethasone. However, a dose of equal to or less than about 375 μg within the ranges disclosed herein is particularly suitable for the present invention.

In certain embodiments, the glucocorticoid, such as dexamethasone, may be contained in the insert of the invention such that particles of the glucocorticoid are dispersed or distributed in a hydrogel comprised of a polymer network. In certain embodiments, the particles are homogeneously dispersed in the hydrogel. The hydrogel may prevent the drug particles from agglomerating and may provide a matrix for the particles which releases the drug in a sustained manner upon contact with the tear fluid.

In certain embodiments of the invention, the glucocorticoid particles, such as the dexamethasone particles, may be microencapsulated. The term “microcapsule” is sometimes defined as a roughly spherical particle with a size varying between e.g. about 50 nm to about 2 mm. Microcapsules have at least one discrete domain (or core) of active agent encapsulated in a surrounding or partially surrounding material, sometimes also referred to as a shell. A suitable agent for microencapsulating the glucocorticoid, such as the dexamethasone, for the purposes of the present invention, is poly(lactic-co-glycolic acid).

In one embodiment, the glucocorticoid particles, such as the dexamethasone particles, may have a small particle size and may be micronized particles. In another embodiment, the glucocorticoid particles, such as the dexamethasone particles, may not be micronized. Micronization refers to the process of reducing the average diameter of particles of a solid material. Particles with reduced diameters may have inter alia higher dissolution rates, which increases the bioavailability of active pharmaceutical ingredients. In the composite materials field, particle size is known to affect the mechanical properties when combined with a matrix, with smaller particles providing superior reinforcement for a given mass fraction. Thus, a hydrogel matrix within which micronized glucocorticoid particles are dispersed may have improved mechanical properties (e.g. brittleness, strain to failure, etc.) compared to a similar mass fraction of larger glucocorticoid particles. Such properties are important in manufacturing, during administration, and during degradation of the insert. Micronization may also promote a more homogeneous distribution of the active ingredient in the chosen dosage form or matrix. In certain embodiments, for any glucocorticoid used in the present invention, including dexamethasone, particle sizes (e.g. as expressed by the d90 value as defined herein and that are measured as also disclosed herein) of about 100 μm or below, or of about 75 μm or below, or of about 50 μm or below may be used. In particular embodiments, dexamethasone may be used in the form of micronized particles and may have a d90 particle size of equal to or less than about 100 μm, or of equal to or less than about 75 μm, or of equal to or less than about 50 μm, or of equal to or less than about 20 μm, or of equal to or less than about 10 μm, or of equal to or less than about 5 μm. In these and other embodiments, the d98 particle size of the micronized dexamethasone may be equal to or less than about 100 μm, or equal to or less than about 75 μm, or equal to or less than about 50 μm, or equal to or less than about 20 μm, or equal to or less than about 10 μm, or equal to or less than about 5 μm. In particular embodiments of the present invention, the micronized dexamethasone used in (or used for preparing) an insert of the present invention has a d90 particle size of equal to or less than about 5 μm and a d98 particle size of less than about 10 μm. In embodiments in which another glucocorticoid than dexamethasone is used in the present invention similar particle sizes may apply as disclosed for dexamethasone.

In certain embodiments, to reduce the presence of discrete particles in the glucocorticoid, in particular the dexamethasone, starting material that are larger than a certain size, such as larger than about 120 μm, or larger than about 100 μm, or larger than about 90 μm, the bulk glucocorticoid material meeting the (d90 and/or d98) particle size specification(s) as disclosed herein may be sieved prior to preparing the wet composition of the insert. In particular embodiments, the dexamethasone used for manufacturing the inserts according to the present invention has a d90 particle size of equal to or less than about 5 μm, and a d98 particle size of less than about 10 μm, with all or essentially all discrete particles having a size of less than about 90 μm.

Micronized dexamethasone particles may be purchased per specification from the supplier (e.g. from Pfizer or Sanofi), or may be prepared according to any of the processes known in the art. For example, micronization processes may be used as e.g. exemplarily disclosed for certain glucocorticoids in EP 2043698 A2 or in EP 2156823 A1 (which are incorporated herein by reference), or processes that are analogous to an exemplary procedure as e.g. disclosed in WO 2016/183296 A1 (which is incorporated herein by reference), Example 13, with respect to a different active agent.

### The Polymer Network

In certain embodiments, the hydrogel may be formed from precursors having functional groups that form crosslinks to create a polymer network. These crosslinks between polymer strands or arms may be chemical (i.e., may be covalent bonds) and/or physical (such as ionic bonds, hydrophobic association, hydrogen bridges etc.) in nature.

The polymer network may be prepared from precursors, either from one type of precursor or from two or more types of precursors that are allowed to react. Precursors are chosen in consideration of the properties that are desired for the resultant hydrogel. There are various suitable precursors for use in making the hydrogels. Generally, any pharmaceutically acceptable and crosslinkable polymers forming a hydrogel may be used for the purposes of the present invention. The hydrogel and thus the components incorporated into it, including the polymers used for making the polymer network, should be physiologically safe such that they do not elicit e.g. an immune response or substantial immune response or other adverse effects. Hydrogels may be formed from natural, synthetic, or biosynthetic polymers.

Natural polymers may include glycosaminoglycans, polysaccharides (e.g. dextran), polyaminoacids and proteins or mixtures or combinations thereof, while this list is not intended to be limiting.

Synthetic polymers may generally be any polymers that are synthetically produced from a variety of feedstocks by different types of polymerization, including free radical polymerization, anionic or cationic polymerization, chain-growth or addition polymerization, condensation polymerization, ring-opening polymerization etc. The polymerization may be initiated by certain initiators, by light and/or heat, and may be mediated by catalysts. Synthetic polymers may in certain embodiments be used to lower the potential of allergies in dosage forms that do not contain any ingredients from human or animal origin.

Generally, for the purposes of the present invention one or more synthetic polymers of the group comprising one or more units of polyalkylene glycol, particularly including but not limited to polyethylene glycol (PEG), polyalkylene oxide such as polyethylene oxide, polypropylene oxide, polyvinyl alcohol, poly (vinylpyrrolidinone), polylactic acid, polylactic-co-glycolic acid, random or block copolymers or combinations/mixtures of any of these can be used, while this list is not intended to be limiting.

To form covalently crosslinked polymer networks, the precursors may be covalently crosslinked with each other. In certain embodiments, precursors with at least two reactive centers (for example, in free radical polymerization) can serve as crosslinkers since each reactive group can participate in the formation of a different growing polymer chain.

The precursors may have biologically inert and hydrophilic portions, e.g., a core. In the case of a branched polymer, a core refers to a contiguous portion of a molecule joined to arms that extend from the core, where the arms carry a functional group, which is often at the terminus of the arm or branch. Multi-armed PEG precursors are examples of such precursors and are used in particular embodiments of the present invention as further disclosed herein.

A hydrogel for use in the present invention can be made e.g. from one multi-armed precursor with a first (set of) functional group(s) and another (e.g. multi-armed) precursor having a second (set of) functional group(s). By way of example, a multi-armed precursor may have hydrophilic arms, e.g., polyethylene glycol units, terminated with primary amines (nucleophile), or may have activated ester end groups (electrophile). The polymer network according to the present invention may contain identical or different polymer units crosslinked with each other. The precursors may be high-molecular weight components (such as polymers having functional groups as further disclosed herein) or low-molecular weight components (such as low-molecular amines, thiols, esters etc. as also further disclosed herein).

Certain functional groups can be made more reactive by using an activating group. Such activating groups include (but are not limited to) carbonyldiimidazole, sulfonyl chloride, aryl halides, sulfosuccinimidyl esters, N-hydroxysuccinimidyl (abbreviated as “NHS”) ester, succinimidyl ester, benzotriazolyl ester, thioester, epoxide, aldehyde, maleimides, imidoesters, acrylates and the like. The NHS esters are useful groups for crosslinking with nucleophilic polymers, e.g., primary amine-terminated or thiol-terminated polyethylene glycols or other nucleophilic group-containing agents, such as nucleophilic group-containing crosslinking agents. An NHS-amine crosslinking reaction may be carried out in aqueous solution and in the presence of buffers, e.g., phosphate buffer (pH 5.0-7.5), triethanolamine buffer (pH 7.5-9.0), borate buffer (pH 9.0-12), or sodium bicarbonate buffer (pH 9.0-10.0).

In certain embodiments, each precursor may comprise only nucleophilic or only electrophilic functional groups, so long as both nucleophilic and electrophilic precursors are used in the crosslinking reaction. Thus, for example, if a crosslinker has only nucleophilic functional groups such as amines, the precursor polymer may have electrophilic functional groups such as N-hydroxysuccinimides. On the other hand, if a crosslinker has electrophilic functional groups such as sulfosuccinimides, then the functional polymer may have nucleophilic functional groups such as amines or thiols. Thus, functional polymers such as proteins, poly (allyl amine), or amine-terminated di-or multifunctional poly(ethylene glycol) can be also used to prepare the polymer network of the present invention.

In one embodiment of the present invention a precursor for the polymer network forming the hydrogel in which the glucocorticoid is dispersed to form the insert according to the present invention has about 2 to about 16 nucleophilic functional groups each (termed functionality), and in another embodiment a precursor has about 2 to about 16 electrophilic functional groups each (termed functionality). Reactive precursors having a number of reactive (nucleophilic or electrophilic) groups as a multiple of 4, thus for example 4, 8 and 16 reactive groups, are particularly suitable for the present invention. However, any number of functional groups, such as including any of 2, 3, 4, 5, 6, 7, 8, 9, 10, 11, 12, 13, 14, 15, or 16 groups, is possible for precursors to be used in accordance with the present invention, while ensuring that the functionality is sufficient to form an adequately crosslinked network.

### PEG Hydrogels

In certain embodiments of the present invention, the polymer network forming the hydrogel contains polyethylene glycol (“PEG”) units. PEGs are known in the art to form hydrogels when crosslinked, and these PEG hydrogels are suitable for pharmaceutical applications e.g. as matrix for drugs intended to be administered to any part of the human or animal body.

The polymer network of the hydrogel inserts of the present invention may comprise one or more multi-arm PEG units having from 2 to 10 arms, or from 4 to 8 arms, or 4, 5, 6, 7 or 8 arms. In certain embodiments, the PEG units used in the hydrogel of the present invention have 4 arms. In certain embodiments, the PEG units used in the hydrogel of the present invention have 8 arms. In certain embodiments, PEG units having 4 arms and PEG units having 8 arms are used in the hydrogel of the present invention. In certain particular embodiments, one or more 4-armed PEGs is/are utilized. Any combination of multi-armed PEGs may be used. In specific embodiments, only 4-arm PEG units are used (which may be the same or different).

The number of arms of the PEG(s) used contributes to controlling the flexibility or softness of the resulting hydrogel. For example, hydrogels formed by crosslinking 4-arm PEGs are generally softer and more flexible than those formed from 8-arm PEGs of the same molecular weight. In particular, if stretching the hydrogel prior to (or also after) drying as disclosed herein below in the section relating to the manufacture of the insert is desired, a more flexible hydrogel may be used, such as a 4-arm PEG, optionally in combination with another multi-arm PEG, such as an 8-arm PEG as disclosed above, or another (different) 4-arm PEG.

In certain embodiments of the present invention, polyethylene glycol units used as precursors have an average molecular weight (Mn) in the range from about 2,000 to about 100,000 Daltons, or in a range from about 10,000 to about 60,000 Daltons, or in a range from about 15,000 to about 50,000 Daltons. In certain particular embodiments the polyethylene glycol units have an average molecular weight in a range from about 10,000 to about 40,000 Daltons, or in a range from about 15,000 to about 30,000 Daltons, or in a range from about 15,000 to about 25,000 Daltons. In specific embodiments, the polyethylene glycol units used for making the hydrogels according to the present invention have an average molecular weight (Mn) of about 20,000 Daltons. Polyethylene glycol precursors of different molecular weight may be combined with each other. When referring herein to a PEG material having a particular average molecular weight (as defined herein), such as about 20,000 Daltons, a variance of ±10% is intended to be included, i.e., referring to a material having an average molecular weight of about 20,000 Daltons also refers to such a material having an average molecular weight of about 18,000 to about 22,000 Daltons. As used herein, the abbreviation “k” in the context of the molecular weight refers to 1,000 Daltons, i.e., “20 k” means 20,000 Daltons.

Further, when referring to a PEG precursor having a certain average molecular weight, such as a 15 kPEG- or a 20 kPEG-precursor, the indicated average molecular weight (i.e., a Mn of 15,000 or 20,000, respectively) refers to the PEG part of the precursor, before end groups are added (“20 k” here means 20,000 Daltons, and “15 k” means 15,000 Daltons—the same abbreviation is used herein for other average molecular weights of PEG precursors). In certain embodiments, the Mn of the PEG part of the precursor is determined by MALDI. The degree of substitution with end groups as disclosed herein may be determined by means of ¹H-NMR after end group functionalization.

In a 4-arm (“4a”) PEG, in certain embodiments each of the arms may have an average arm length (or molecular weight) of the total molecular weight of the PEG divided by 4. A 4a 20 kPEG precursor, which is a particularly suitably precursor for use in the present invention thus has 4 arms with an average molecular weight of about 5,000 Daltons each and a total molecular weight of 20,000 Daltons. An 8a 20 k PEG precursor, which could also be used in combination with or alternatively to the 4a 20 kPEG precursor in the present invention, thus has 8 arms (“8a”) each having an average molecular weight of 2,500 Daltons and a total molecular weight of 20,000 Daltons. Longer arms may provide increased flexibility as compared to shorter arms. PEGs with longer arms may swell more as compared to PEGs with shorter arms. A PEG with a lower number of arms also may swell more and may be more flexible than a PEG with a higher number of arms. In certain particular embodiments, only one or more 4-arm PEG precursor(s) is/are utilized in the present invention. In certain other embodiments, a combination of one or more 4-arm PEG precursor(s) and one or more 8-arm PEG precursor(s) is utilized in the present invention. In addition, longer PEG arms have higher melting temperatures when dry, which may provide more dimensional stability during storage.

In certain embodiments, electrophilic end groups for use with PEG precursors for preparing the hydrogels of the present invention are N-hydroxysuccinimidyl (NHS) esters, including but not limited to NHS dicarboxylic acid esters such as the succinimidylmalonate group, succinimidylmaleate group, succinimidylfumarate group, “SAZ” referring to a succinimidylazelate end group, “SAP” referring to a succinimidyladipate end group, “SG” referring to a succinimidylglutarate end group, and “SS” referring to a succinimidylsuccinate end group. Examples of other activated esters in addition to the NHS esters that are useful in the present invention are (without being limited to these) thioesters, benzotriazolyl esters, and esters of acrylic acids.

In certain embodiments, nucleophilic end groups for use with electrophilic group-containing PEG precursors for preparing the hydrogels of the present invention are amine (denoted as “NH₂”) end groups. Thiol (—SH) end groups or other nucleophilic end groups are also possible.

In certain embodiments of the present invention, 4-arm PEGs with an average molecular weight of about 20,000 Daltons and electrophilic end groups as disclosed above (such as the SAZ, SAP, SG and SS end groups, particularly the SG end group) are crosslinked for forming the polymer network and thus the hydrogel according to the present invention. Suitable PEG precursors are available from a number of suppliers, such as Jenkem Technology and others.

Reactions of e.g. nucleophilic group-containing crosslinkers and electrophilic group-containing PEG units, such as reaction of amine group-containing crosslinkers with activated ester-group containing PEG units, result in a plurality of PEG units being crosslinked by a hydrolyzable linker having the formula:

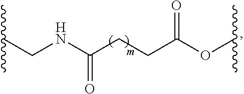

wherein m is an integer from 0 to 10, and specifically is 1, 2, 3, 4, 5, 6, 7, 8, 9, or 10. For a SAZ-end group, m would be 6, for a SAP-end group, m would be 3, for a SG-end group, m would be 2 and for an SS-end group, m would be 1. In particular embodiments, m is 2. All crosslinks within the polymer network may be the same, or may be different.

In certain embodiments, the polymer precursors used for forming the hydrogel according to the present invention may be selected from 4a 20 kPEG-SAZ, 4a 20 kPEG-SAP, 4a 20 kPEG-SG, 4a 20 kPEG-SS, 8a 20 kPEG-SAZ, 8a 20 kPEG-SAP, 8a 20 kPEG-SG, 8a 20 kPEG-SS, or mixtures thereof, with one or more PEG- or lysine based-amine groups selected from 4a 20 kPEG-NH₂, 8a 20 kPEG-NH₂, and trilysine, or a trilysine salt or derivative, such as trilysine acetate.

In certain embodiments, the SG end group is utilized in the present invention. This end group may provide for a shorter time until the hydrogel is biodegraded in an aqueous environment such as in the tear fluid, when compared to the use of other end groups, such as the SAZ end group, which provides for a higher number of carbon atoms in the linker and may thus be more hydrophobic and therefore less prone to ester hydrolysis than the SG end group.

In particular embodiments, a 4-arm 20,000 Dalton PEG precursor having a SG end group (as defined above), is crosslinked with a crosslinking agent having one or more reactive amine end groups. This PEG precursor is abbreviated herein as 4a 20 kPEG-SG. A schematic chemical structure of 4a 20 kPEG-SG is reproduced below:

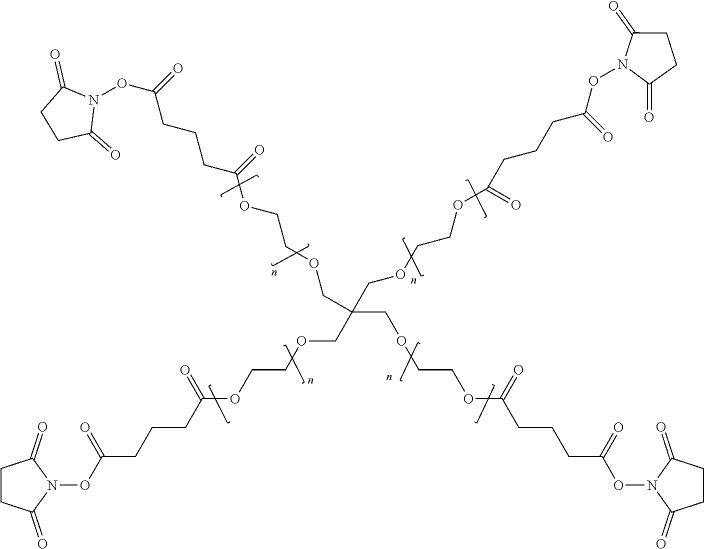

In this formula, n is determined by the molecular weight of the respective PEG-arm.

In certain particular embodiments, the crosslinking agent (herein also referred to as “crosslinker”) used is a low-molecular weight component containing nucleophilic end groups, such as amine or thiol end groups. In certain embodiments, the nucleophilic group-containing crosslinking agent is a small molecule amine with a molecular weight below 1,000 Da. In certain embodiments, the nucleophilic-group containing crosslinking agent comprises two, three or more primary aliphatic amine groups. Suitable crosslinking agents for use in the present invention are (without being limited to) spermine, spermidine, lysine, dilysine, trilysine, tetralysine, polylysine, ethylenediamine, polyethylenimine, 1,3-diaminopropane, 1,3-diaminopropane, diethylenetriamine, trimethylhexamethylenediamine, 1,1,1-tris(aminoethyl)ethane, their pharmaceutically acceptable salts, hydrates or other solvates and their derivatives such as conjugates (as long as sufficient nucleophilic groups for crosslinking remain present), and any mixtures thereof. A particular crosslinking agent for use in the present invention is a lysine-based crosslinking agent, such as trilysine or a trilysine salt or derivative. A particular nucleophilic crosslinking agent for use in the present invention is trilysine acetate. Other low-molecular weight multi-arm amines may be used as well. The chemical structure of trilysine is reproduced below:

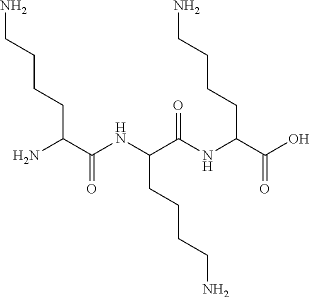 [SMILES: NCCCCC(N)C(=O)NC(CCCCN)C(=O)NC(CCCCN)C(=O)O]

In very particular embodiments of the present invention, a 4a 20 kPEG-SG precursor is reacted with trilysine acetate, to form the polymer network.

In certain embodiments, the nucleophilic group-containing crosslinking agent is bound to or conjugated with a visualization agent. Fluorophores such as fluorescein, rhodamine, coumarin, and cyanine can be used as visualization agents as disclosed herein. In specific embodiments of the present invention, fluorescein is used as the visualization agent. The visualization agent may be conjugated with the crosslinking agent e.g. through some of the nucleophilic groups of the crosslinking agent. Since a sufficient amount of the nucleophilic groups are necessary for crosslinking, “conjugated” or “conjugation” in general includes partial conjugation, meaning that only a part of the nucleophilic groups may be used for conjugation with the visualization agent, such as about 1% to about 20%, or about 5% to about 10%, or about 8% of the nucleophilic groups of the crosslinking agent may be conjugated with a visualization agent. In specific embodiments, the crosslinking agent is trilysine acetate and is conjugated with fluorescein.

In other embodiments, the visualization agent may also be conjugated with the polymer precursor, e.g. through certain reactive (such as electrophilic) groups of the polymer precursors. In certain embodiments, the crosslinking agent itself or the polymer precursor itself may contain an e.g. fluorophoric or other visualization-enabling group.

In the present invention, conjugation of the visualization agent to either the polymer precursor(s) or to the crosslinking agent as disclosed below is intended to keep the visualization agent in the hydrogel while the active agent is released into the tear fluid, thus allowing confirmation of insert presence within the canaliculus by a convenient, non-invasive method.

In certain embodiments, the molar ratio of the nucleophilic and the electrophilic end groups reacting with each other is about 1:1, i.e., one amine group is provided per one electrophilic, such as SG, group. In the case of 4a 20 kPEG-SG and trilysine (acetate) this results in a molar ratio of the two components of about 1:1 as the trilysine has four primary amine groups that may react with the electrophilic SG ester group. However, an excess of either the electrophilic (e.g. NHS, such as the SG) end group precursor or of the nucleophilic (e.g. the amine) end group precursor may be used. In particular, an excess of the nucleophilic, such as the amine end group containing precursor or crosslinking agent may be used. In certain embodiments, the molar ratio of the electrophilic group containing precursor to the nucleophilic group-containing crosslinking agent, such as the molar ratio of 4a 20 kPEG-SG to trilysine acetate, is from about 1:2 to about 2:1.

Finally, in alternative embodiments the amine linking agent can also be another PEG precursor with the same or a different number of arms and the same or a different arm length (average molecular weight) as the 4a 20 kPEG-SG, but having terminal amine groups, i.e., 4a 20 kPEG-NH₂.

### Additional Ingredients

The insert of the present invention may contain, in addition to the polymer units forming the polymer network as disclosed above and the active principle, other additional ingredients. Such additional ingredients are for example salts originating from buffers used during the preparation of the hydrogel, such as phosphates, borates, bicarbonates, or other buffer agents such as triethanolamine. In certain embodiments of the present invention sodium phosphate buffers (specifically, mono- and dibasic sodium phosphate) are used.

In a specific embodiment, the insert of the present invention is free of anti-microbial preservatives or at least does not contain a substantial amount of anti-microbial preservatives (including, but not limited to benzalkonium chloride (BAK), chlorobutanol, sodium perborate, and stabilized oxychloro complex (SOC)).

In a further specific embodiment, the insert of the present invention does not contain any ingredients of animals or human origin but only contains synthetic ingredients.

In certain embodiments, the inserts of the present invention contain a visualization agent. Visualization agents to be used according to the present invention are all agents that can be conjugated with the components of the hydrogel or can be entrapped within the hydrogel, and that are visible, or may be made visible when exposed e.g. to light of a certain wavelength, or that are contrast agents. Suitable visualization agents for use in the present invention are (but are not limited to) e.g. fluoresceins, rhodamines, coumarins, cyanines, europium chelate complexes, boron dipyromethenes, benzofrazans, dansyls, bimanes, acridines, triazapentalenes, pyrenes and derivatives thereof. Such visualization agents are commercially available e.g. from TCI. In certain embodiments the visualization agent is a fluorophore, such as fluorescein or comprises a fluorescein moiety. Visualization of the fluorescein-containing insert is possible by illumination with blue light and a yellow filter. The fluorescein in the intracanalicular insert illuminates when excited with blue light enabling confirmation of insert presence. In specific embodiments, the visualization agent is conjugated with one of the components forming the hydrogel. For example, the visualization agent, such as fluorescein, is conjugated with the crosslinking agent, such as the trilysine or trilysine salt or derivate (e.g. the trilysine acetate), or with the PEG-component. For example, NHS-fluorescein may be conjugated with trilysine acetate prior to the crosslinking reaction with the PEG precursor(s). Conjugation of the visualization agent prevents the visualization agent from being eluted or released out of the insert. A method of conjugating a visualization agent with the crosslinking agent is illustrated in Example 1. Since a sufficient amount of the nucleophilic groups (at least more than one molar equivalent) are necessary for crosslinking, partial conjugation of the visualization agent with e.g. the crosslinking agent as disclosed above may be performed.

The insert of the present invention may in certain embodiments contain a surfactant. The surfactant may be a non-ionic surfactant. The non-ionic surfactant may comprise a poly(ethylene glycol) chain. Exemplary non-ionic surfactants are poly(ethylene glycol) sorbitan monolaurate commercially available as Tween® (and in particular Tween®20, a PEG-20-sorbitan monolaurate, or Tween®80, a PEG-80-sorbitan monolaurate), poly(ethylene glycol) ester of castor oil commercially available as Cremophor (and in particular Cremophor40, which is PEG-40-castor oil), and an ethoxylated 4-tert-octylphenol/formaldehyde condensation polymer which is commercially available as Tyloxapol and others such as Triton. A surfactant may aid in dispersing the active principle and may prevent particle aggregation, and may also reduce possible adhesion of the hydrogel strand to the tubing during drying.

### Formulation

In certain embodiments, inserts according to the present invention comprise a glucocorticoid, such as dexamethasone, a polymer network made from one or more polymer precursors as disclosed herein in the form of a hydrogel, and optional additional components such as visualization agents, salts etc. remaining in the insert from the production process (such as phosphate salts used as buffers etc.). In certain preferred embodiments, the glucocorticoid is dexamethasone. In particular embodiments, the insert is preservative-free.

In some embodiments, the inserts according to the present invention in a dry state contain from about 30% to about 70% by weight glucocorticoid, such as dexamethasone, and from about 25% to about 60% by weight polymer units, such as those disclosed above. In further embodiments, the inserts according to the present invention in a dry state contain from about 30% to about 60% by weight glucocorticoid, such as dexamethasone, and from about 30% to about 60% by weight polymer units, such as those disclosed above.

In certain embodiments, the inserts according to the present invention in a dry state contain from about 40% to about 56% by weight glucocorticoid, such as dexamethasone, and from about 36% to about 55% by weight polymer units, such as polyethylene glycol units as disclosed above.

In certain particular embodiments, the inserts according to the present invention in a dry state contain from about 40% to about 46% by weight dexamethasone and from about 45% to about 55% by weight PEG units.

In certain other particular embodiments, the inserts according to the present invention in a dry state contain from about 50% to about 56% by weight dexamethasone and from about 36% to about 46% by weight PEG units.

In certain embodiments, the inserts according to the present invention may contain in a dry state about 0.1% to about 1% by weight visualization agent, such as fluorescein or a molecule comprising a fluorescein moiety. Also in certain embodiments, the inserts according to the present invention may contain in a dry state about 0.5% to about 5% by weight of one or more buffer salt(s) (separately or taken together). In certain embodiments, the insert in a dry state may contain, e.g., from about 0.01% to about 2% by weight or from about 0.05% to about 0.5% by weight of a surfactant.

In a particular embodiment, an insert according to the present invention may be made from about 200 μg dexamethasone (i.e., containing a target dose of about 200 μg dexamethasone within a variance of +25% and −20%, or within a variance of ±10%, as disclosed herein), and about 200 μg to about 250 μg PEG-units, such as 4a 20 kPEG-SG, about 5 μg to about 7 μg trilysine acetate, about 1 μg to about 3 μg visualization agent, such as fluorescein, and about 2.5 μg to about 20 μg buffer salt, such as sodium phosphate (mono- and/or dibasic).

In another particular embodiment, an insert according to the present invention may be made from about 300 μg dexamethasone (i.e., containing a target dose of about 300 μg dexamethasone within a variance of +25% and −20%, or within a variance of ±10%, as disclosed herein), and about 200 μg to about 250 μg PEG-units, such as 4a 20 kPEG-SG, about 5 μg to about 7 μg trilysine acetate, about 1 μg to about 3 μg visualization agent, such as fluorescein and about 2.5 μg to about 20 μg buffer salt, such as sodium phosphate (mono- and/or dibasic).

In certain embodiments, the balance of the insert in its dry state (i.e., the remainder of the formulation when glucocorticoid, such as dexamethasone, and polymer hydrogel, such as trilysine-crosslinked PEG hydrogel, and optionally visualization agent, such as fluorescein, have already been taken account of) may be salts remaining from the buffer used during manufacture of the inserts as disclosed herein, or may be other ingredients used during manufacturing of the insert (such as surfactants if used). In certain embodiments, such salts are phosphate, borate or (bi) carbonate salts. In one embodiment a buffer salt is sodium phosphate (mono- and/or dibasic).

The amounts of the glucocorticoid and the polymer(s) may be varied, and other amounts of the glucocorticoid and the polymer hydrogel than those disclosed herein may also be used to prepare inserts according to the invention.

In certain embodiments, the maximum amount (in weight %) of drug within the formulation is about two times the amount of the polymer (e.g., PEG) units, but may be higher in certain cases, as long as the mixture comprising e.g., the precursors, visualization agent, buffers and drug (in the state before the hydrogel has gelled completely) can be uniformly cast into a desired mold or thin-diameter tubing and/or the hydrogel is still sufficiently stretchable as disclosed herein, and/or sufficiently increases in diameter upon hydration as also disclosed herein.

In certain embodiments, solid contents of about 20% to about 50% (w/v) (wherein “solids” means the combined weight of polymer precursor(s), optional visualization agent, salts and the drug in solution) are utilized for forming the hydrogel of the inserts according to the present invention.

In certain embodiments, the water content of the hydrogel in a dry (dehydrated/dried) state may be low, such as not more than about 1% by weight of water (determined e.g. as disclosed herein). The water content may in certain embodiments also be lower than that, possibly no more than about 0.25% by weight or even no more than about 0.1% by weight.

### Dimensions of the Insert and Dimensional Change upon Hydration

The dried insert may have different geometries, depending on the method of manufacture, such as the inner diameter or shape of a mold or tubing into which the mixture comprising the hydrogel precursors including the glucocorticoid is cast prior to complete gelling. The insert according to the present invention is also referred as a “fiber” (which term is used interchangeably herein with the term “rod”), wherein the fiber in general has a length that exceeds its diameter. The insert (or the fiber) may have different geometries, with specific dimensions as disclosed herein.

In one embodiment, the insert is cylindrical or has an essentially cylindrical shape. Whenever in the specification or in the claims it is herein referred to “cylindrical” in the context of the shape of the insert, this always includes “essentially cylindrical”. In this case, the insert has a round or an essentially round cross-section. In other embodiments of the invention, the insert is non-cylindrical. The insert according to the present invention is optionally elongated in its dry state, wherein the length of the insert is greater than the width of the insert, wherein the width is the largest cross sectional dimension that is substantially perpendicular to the length. In a cylindrical or essentially cylindrical insert, the width is also referred to as the diameter.

Various geometries of the outer insert shape or its cross-section may also be used in the present invention. For example, instead of a round diameter fiber (i.e., in the case of a cylindrical insert), an oval (or elliptical) diameter fiber may be used. Other cross-sectional geometries, such as oval or oblong, rectangular, triangular, star-shaped, cross-shaped etc. may generally be used. As long as the insert expands in diameter upon hydration in the canaliculus to a hydrated diameter as disclosed herein, the exact cross-sectional shape is not decisive, as tissue will form around the insert. In certain embodiments, the ratio of the length of the insert to the diameter of the insert in the hydrated state is at least about 1, or at least about 1.1, or at least about 1.2, which aids in keeping the insert in place in the canaliculus and prevents the insert from twisting and turning within the canaliculus, and also aids in maintaining a close contact with surrounding tissue. In certain embodiments, this ratio may be less than about 2, or less than about 1.75.

The polymer network, such as the PEG network, of the hydrogel insert according to certain embodiments of the present invention may be semi-crystalline in the dry state at or below room temperature, and amorphous in the wet state. Even in the stretched form, the dry insert may be dimensionally stable at or below room temperature, which may be advantageous for administering the insert into the canaliculus, and also for quality control.

Upon hydration of the insert in the canaliculus by the tear fluid (which can be simulated in vitro e.g. by immersing the insert into PBS, pH 7.4 at 37° C. after 24 hours, which is considered equilibrium) the dimensions of the insert according to the invention may change. Generally, the diameter of the insert may increase, while its length may decrease or in certain embodiments may stay the same or essentially the same. An advantage of this dimensional change is that, while the insert in its dry state is sufficiently thin to be administered and placed into the canaliculus through the punctum (which itself is smaller in diameter than the canaliculus) upon hydration and thereby through expansion of its diameter it fits closely into the canaliculus and thus acts as a canalicular plug. The insert therefore provides for lacrimal occlusion and thereby tear conservation in addition to releasing the active principle in a controlled manner to the tear fluid over a certain period of time as disclosed herein.

In certain embodiments, this dimensional change is enabled at least in part by the “shape memory” effect introduced into the insert by means of stretching the hydrogel strand in the longitudinal direction during its manufacture as also disclosed herein. In certain embodiments, this stretching may be performed in the wet state, i.e., before drying. However, in certain other embodiments, the stretching of the hydrogel strands (once casted and cured) may be performed in the dry state (i.e., after drying the hydrogel strands). It is noted that if no stretching is performed at all the insert may merely swell due to the uptake of water, but the dimensional change of an increase in diameter and a decrease in length disclosed herein may not be achieved, or may not be achieved to a large extent. This could result in a less than optimal fixture of the insert in the canaliculus, and could potentially lead to the insert being cleared (potentially even prior to the release of the complete dose of the active principle) through the nasolacrimal duct or through the punctum. If this is not desired, the hydrogel strand may e.g. be dry or wet stretched in order to provide for expansion of the diameter upon rehydration.

In the hydrogels of the present invention, a degree of molecular orientation may be imparted by stretching the material then allowing it to solidify, locking in the molecular orientation. The molecular orientation provides one mechanism for anisotropic swelling upon contacting the insert with a hydrating medium such as tear fluid. Upon hydration, the insert of certain embodiments of the present invention will swell only in the radial dimension, while the length will either decrease or be maintained or essentially maintained. The term “anisotropic swelling” means swelling preferentially in one direction as opposed to another, as in a cylinder that swells predominantly in diameter, but does not appreciably expand (or does even contract) in the longitudinal dimension.

The degree of dimensional change upon hydration may depend inter alia on the stretch factor. Merely as an example to illustrate the effect of stretching, stretching at e.g. a stretch factor of about 1.3 (e.g. by means of wet stretching) may have a less pronounced effect or may not change the length and/or the diameter during hydration to a large extent. In contrast, stretching at e.g. a stretch factor of about 1.8 (e.g. by means of wet stretching) may result in a shorter length and/or an increased diameter during hydration. Stretching at e.g. a stretch factor of about 3 or 4 (e.g. by means of dry stretching) could result in a much shorter length and a much larger diameter upon hydration. One skilled in the art will appreciate that other factors besides stretching can also affect swelling behavior.

Among other factors influencing the possibility to stretch the hydrogel and to elicit dimensional change of the insert upon hydration is the composition of the polymer network. In the case PEG precursors are used, those with a lower number of arms (such as 4-armed PEG precursors) contribute to providing a higher flexibility in the hydrogel than those with a higher number of arms (such as 8-armed PEG precursors). If a hydrogel contains more of the less flexible components (e.g. a higher amount of PEG precursors containing a larger number of arms, such as the 8-armed PEG units), the hydrogel may be firmer and less easy to stretch without fracturing. On the other hand, a hydrogel containing more flexible components (such as PEG precursors containing a lower number of arms, such as 4-armed PEG units) may be easier to stretch and softer, but also swells more upon hydration. Thus, the behavior and properties of the insert once it has been administered and is rehydrated can be tailored by means of varying structural features as well as by modifying the processing of the insert after it has been initially formed.

The dried insert dimensions inter alia may depend on the amount of glucocorticoid incorporated as well as the ratio of glucocorticoid to polymer units and can additionally be controlled by the diameter and shape of the mold or tubing in which the hydrogel is allowed to gel. The diameter of the dried insert may be further controlled by (wet or dry) stretching of the hydrogel strands once formed as disclosed herein. The dried hydrogel strands (after stretching) are cut into segments of the desired length to form the insert; the length can thus be chosen as desired.

In the following, embodiments of inserts with specific dimensions are disclosed. The dimensional ranges or values disclosed in the specific embodiments herein relate to the length and the diameter of cylindrical or essentially cylindrical inserts. However, all values and ranges for cylindrical inserts may also be used correspondingly for non-cylindrical inserts. In case several measurements of the length or diameter of one insert are conducted, or several datapoints are collected during the measurement, the average (i.e., mean) value is reported as defined herein. The length and diameter of an insert according to the invention may be measured e.g. by means of microscopy, or by means of an (optionally automated) camera system, e.g. as disclosed in Example 1. Other suitable methods of measuring insert dimensions may also be used.

In one embodiment, the present invention relates to a sustained release biodegradable intracanalicular insert comprising a hydrogel and a glucocorticoid, wherein the insert in a dry state has a length of equal to or less than about 2.75 mm. In a particular embodiment, the glucocorticoid is dexamethasone.

In certain embodiments of the invention, the insert in a dry state has a length of equal to or less than about 2.5 mm, or less than about 2.3 mm, or has a length of about 2.25 mm. In certain embodiments of the invention, the insert in a dry state has a length of greater than about 1 mm, or greater than about 1.5 mm, or greater than about 2 mm. In certain particular embodiments, the insert in its dry state has a length of less than about 2.5 mm and greater than about 1.5 mm.

In alternative embodiments, the insert may have a length of about 0.5 mm to about 3 mm (e.g., about 0.5 mm to about 2.5 mm, about 1 mm to about 2.5 mm, about 1.25 mm to about 2.5 mm, about 1.5 mm to about 2.25 mm, about 0.5 mm, about 0.75 mm, about 1 mm, about 1.25 mm, about 1.5 mm, about 1.75 mm, about 2.0 mm, about 2.25 mm, about 2.5 mm, about 2.75 mm, or about 3 mm).

In certain embodiments of the invention, the insert in a dry state has a diameter of less than about 1 mm, or less than about 0.8 mm, or less than about 0.75 mm, or less than about 0.6 mm, or a diameter from about 0.40 mm to about 0.58 mm, or of about 0.45 mm, or of about 0.5 mm.

In specific embodiments of the invention, the insert in a dry state has a length in the range of about 2.14 mm to about 2.36 mm and a diameter in the range of about 0.41 mm to about 0.55 mm.

In certain embodiments, an insert according to the invention is cylindrical or essentially cylindrical and upon hydration (in vivo in the canaliculus, or in vitro after 24 hours in phosphate-buffered saline at a pH of 7.2 at 37° C.) the diameter of the insert is increased and the length of the insert is decreased. In particular, the diameter of the insert may be increased by a factor in the range of about 1.5 to about 4, or of about 2 to about 3.5, or of about 3. In other words, the ratio of the diameter of the insert in the hydrated state to the diameter of the insert in the dry state may be in the range of about 1.5 to about 4, or of about 2 to about 3.5, or of about 3.

In certain embodiments, the length of an insert according to the invention is decreased after hydration to about 0.9 times its length in the dry state, or to about 0.75 times its length in the dry state, or to about two-thirds of its length in the dry state. In other words, the ratio of the length of the insert in the hydrated state to the length of the insert in the dry state may be about 0.9 or less, or about 0.75 or less, or about two-thirds or less, and may be at least about 0.25, or at least about 0.4.

Thus, in certain embodiments an insert according to the present invention in its hydrated state has a diameter in the range of about 1 to about 2 mm, and a length that is shorter than the length of the insert in its dry state. In certain embodiments, in the hydrated state, e.g. when the insert has been placed into the canaliculus, the ratio of length to diameter of the insert is suitably greater than 1, i.e., the length of the insert is longer than its diameter. This aids in keeping the insert in place in the canaliculus without any twisting or turning. This aids in occluding the canaliculus/the punctum and keeping the tear fluid within the eye, as well as ensuring contact between the surface of the insert and the tear fluid for releasing the glucocorticoid such as dexamethasone.

In certain embodiments, an insert according to the present invention in the hydrated state has a diameter in the range of about 1.35 mm to about 1.80 mm and a length in the range of about 1.65 mm to about 2.0 mm. Specifically, an insert according to the present invention in the hydrated state may have a diameter in the range of about 1.40 mm to about 1.60 mm and a length in the range of about 1.70 mm to about 2.0 mm, such as a diameter of about 1.5 mm and a length of about 1.8 mm.

In certain embodiments, the dimensional change may be achieved by wet stretching the hydrogel strand at a stretch factor in the range of about 1.5 to about 3, or of about 2.2 to about 2.8, or of about 2.5 to about 2.6. In other embodiments, such dimensional change may be achieved by dry stretching.

In certain embodiments, the stretching thus creates a shape memory, meaning that the insert upon hydration when administered into the canaliculus and once it comes into contact with the tear fluid, will shrink in length and widen in diameter until it approaches (more or less) its equilibrium dimensions, which are determined inter alia by the original molded dimensions and compositional variables. While the narrow dry dimensions facilitate administration of the insert through the punctum into the canaliculus, the widened diameter and shortened length after administration yield a shorter but wider insert that fits closely into and occludes the canaliculus while releasing active agent primarily at its proximal surface (the surface of the insert that is in contact with the tear fluid and that is directed toward the punctum opening).

In a certain embodiment, in case the sustained release biodegradable intracanalicular insert of the present invention is defined by a content of dexamethasone of equal to or less than about 375 μg or an equivalent dose of another glucocorticoid (as disclosed herein above) and/or a release of a therapeutically effective amount of a glucocorticoid for a period of up to about 25 days after administration (as disclosed herein below), the length of the insert in a dry state may also exceed 2.75 mm, e.g. the length of the insert in a dry state may be equal to or above about 3 mm. However, a length of equal to or less than about 2.75 mm as disclosed herein is particularly suitable for the present invention.

In one specific embodiment, an insert of the present invention contains about 200 μg dexamethasone (including the variances of +25%/−20% or ±10% as disclosed herein), has an (essentially) cylindrical shape, and has in the dry state a diameter in the range of about 0.41 mm to about 0.49 mm, such as about 0.47 mm, or about 0.45 mm, and a length in the range of about 2.14 mm to about 2.36 mm, such as about 2.25 mm. Such an insert may decrease in length and increase in diameter upon hydration in vivo in the canaliculus or in vitro (wherein hydration in vitro is measured in phosphate-buffered saline at a pH of 7.4 at 37° C. after 24 hours, which is considered equilibrium) to a length that is shorter than its length in the dry state, such as to a length in the range of about 1.69 mm to about a 1.87 mm, such as about 1.79 mm or about 1.8 mm, and to a diameter in the range of about 1.35 mm to about 1.80 mm, such as about 1.5 mm or about 1.54 mm.

In another specific embodiment, an insert of the present invention contains about 300 μg dexamethasone (including the variances of +25%/−20% or ±10% as disclosed herein), has an (essentially) cylindrical shape, and has a diameter in the range of about 0.44 mm to about 0.55 mm, such as about 0.5 mm or about 0.51 mm, and a length in the range of about 2.14 mm to about 2.36 mm, such as about 2.25 mm, in its dry state. Such an insert may decrease in length and increase in diameter upon hydration in vivo in the canaliculus or in vitro (wherein hydration in vitro is measured in phosphate-buffered saline at a pH of 7.4 at 37° C. after 24 hours, which is considered equilibrium) to a length that is shorter than its length in the dry state, such as to a length in the range of about 1.64 mm to about 2.0 mm, such as about 1.8 mm or about 1.85 mm, and to a diameter in the range of about 1.35 mm to about 1.80 mm, such as about 1.5 mm or about 1.47 mm.

In certain embodiments, an insert of the present invention has a total weight in the range of about 100 to about 1000 μg, such as in the range of about 200 to about 800 μg, or in the range of about 300 to about 700 μg. In particular embodiments, an insert of the present invention has a total weight in the range of about 400 to about 600 μg, such as from about 400 to about 500 μg (in particular, if the insert contains dexamethasone in an amount of about 200 μg including the variances as disclosed herein), or from about 500 to about 600 μg (in particular, if the insert contains dexamethasone in an amount of about 300 μg including the variances as disclosed herein).

### Release of the Active and Biodegradation of the Insert

In one embodiment, the present invention relates to a sustained release biodegradable ocular (such as intracanalicular) insert comprising a hydrogel and a glucocorticoid, wherein the insert provides for a release of a therapeutically effective amount of the glucocorticoid for a period of up to about 25 days after administration (i.e., after having been inserted into the canaliculus). In a particular embodiment, the glucocorticoid is dexamethasone.

Without wishing to be bound by theory, release of the glucocorticoid into the tear fluid is determined by the glucocorticoid's solubility in an aqueous environment. One particular glucocorticoid for use according to the present invention is dexamethasone. The solubility of dexamethasone has been determined to be very low in an aqueous medium (less than 100 μg/mL), such as the tear fluid. When administered to the canaliculus, the dexamethasone is released from the insert primarily at its surface proximal to the tear fluid and thus proximal to the eye surface (i.e., at the insert surface facing the punctum opening).

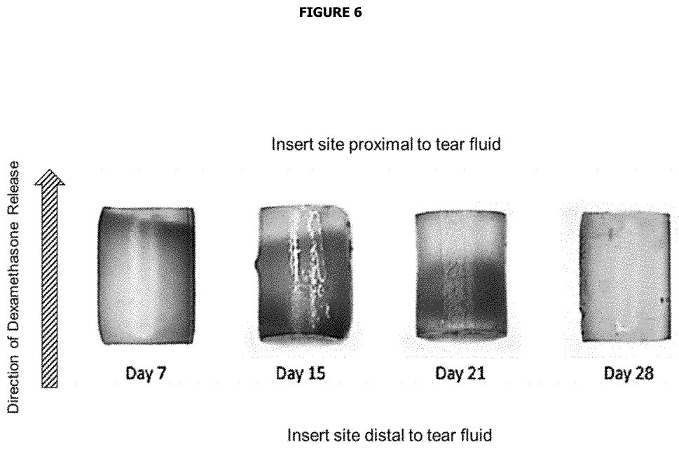

In certain embodiments, the active agent gradually gets dissolved and diffuses out of the hydrogel into the tear fluid. This happens primarily in a unidirectional manner, starting at the interface of the insert and the tear fluid at the proximal surface of the insert. The “drug front” generally progresses in the opposite direction, i.e., away from the proximal surface until eventually the entire insert is depleted of active agent. This is illustrated in FIG. 6.

In certain embodiments, the insert according to the present invention provides for the release of a (therapeutically effective amount of) glucocorticoid, such as dexamethasone, for a period of about 6 hours or longer, such as for a period of about 12 hours or longer, such as for a period of at least about 1 day, or for a period of at least about 7 days, after administration, which is longer than known immediate release ophthalmic dosage forms.

In certain embodiments, the insert according to the present invention provides for the release of a (therapeutically effective amount of) glucocorticoid, such as dexamethasone, for a period of up to about 1 months, or up to about 25 days, or up to about 21 days (i.e., about 3 weeks), or up to about 14 days (i.e., about 2 weeks) after administration.

In certain embodiments, after administration, the levels of active agent released from the insert per day remain sustained, constant or essentially constant over a certain period of time (due to the limitation of release based on the active agent's solubility), such as for about 7 days, or for about 11 days, or for about 14 days in the case of dexamethasone. Then the amount of active agent released per day may decrease for another period of time (also referred to as “tapering”), such as for a period of about 7 additional days (or longer in certain embodiments) in the case of dexamethasone until all or substantially all of the active agent has been released and the “empty” hydrogel remains in the canaliculus until it is fully degraded and/or until it is cleared (disposed/washed out) through the nasolacrimal duct.

In certain embodiments, an insert of the invention provides for an average release of about 5 to about 50 μg, such as about 10 to about 35 μg, specifically about 15 μg to about 25 μg, such as about 20 μg, dexamethasone per day into tear fluid during the period in which the release is sustained, constant or essentially constant, for example during a period of up to about 7 days, or up to about 11 days, or up to about 14 days after administration, or longer.

In specific embodiments, from an insert containing a target dose of about 200 μg (including variances as disclosed herein) dexamethasone, about 15 μg to about 25 μg, such as about 20 μg, dexamethasone are released on average per day into tear fluid for a period of up to 7 days after administration, followed by a period of up to about 7 additional days or longer during which lower amounts of dexamethasone are released until all of the dexamethasone contained in the insert has been released. Overall, an insert containing a target dose of about 200 μg (including variances as disclosed herein) dexamethasone may provide for release of a therapeutically effective amount of dexamethasone into tear fluid for a period of, e.g., up to about 7 days after administration, up to about 14 days after administration, or longer.

In other specific embodiments, from an insert containing a target dose of about 300 μg (including variances as disclosed herein) dexamethasone, about 15 μg to about 25 μg, such as about 20 μg, dexamethasone are released on average per day for a period of up to about 11 days, or up to about 14 days after administration, followed by a period of up to about 7 additional days or longer during which lower amounts of dexamethasone are released until all of the dexamethasone contained in the insert has been released. Overall, an insert containing a target dose of about 300 μg (including variances as disclosed herein) dexamethasone may provide for release of a therapeutically effective amount of dexamethasone for a period of up to about 21 days after administration, or longer.

In a further embodiment, in case the sustained release biodegradable intracanalicular insert of the present invention is defined by a dose of equal to or less than about 375 μg dexamethasone (as disclosed herein above) or an equivalent dose of another glucocorticoid contained in the insert, and/or by a length of equal to or less than about 2.75 mm (as disclosed herein above), the insert may provide for a release of a therapeutically effective amount of a glucocorticoid, e.g., for a period of longer than 25 days after administration, such as up to about 1 month, or even longer, such as up to about 2 months, or up to about 3 months after administration. However, a release of a therapeutically effective amount of dexamethasone (or other glucocorticoid) for a period of up to about 25 days, specifically for up to about 14 days (i.e., about 2 weeks) or up to about 21 days (i.e., about 3 weeks) after administration is particularly suitable for the present invention.

When drug is released primarily from the proximal surface of the insert, this region of the hydrogel insert becomes devoid of drug particles and may therefore also be called the “clearance zone”. In certain embodiments, upon hydration the “clearance zone” is thus a region of the insert that has a concentration of active agent that is less than the active agent in another region of the hydrated hydrogel. As the clearance zone increases, it creates a concentration gradient within the insert that may lead to tapering of the release rate of the drug.

Concurrently with the drug diffusing out of the hydrogel (and also after the entire amount of drug has diffused out of the hydrogel), the hydrogel may be slowly degraded e.g. by means of ester hydrolysis in the aqueous environment of the tear fluid. At advanced stages of degradation, distortion and erosion of the hydrogel begins to occur. As this happens, the hydrogel becomes softer and more liquid (and thus its shape becomes distorted) until the hydrogel finally dissolves and is resorbed completely. However, as the hydrogel becomes softer and thinner and its shape becomes distorted, at a certain point it may no longer remain at its intended site in the canaliculus to which it had been administered, but it may progress deeper into the canaliculus and eventually may be cleared (disposed/washed out) through the nasolacrimal duct.

In one embodiment, the persistence of the hydrogel within an aqueous environment such as in the human eye (including the canaliculus) depends inter alia on the structure of the linker that crosslinks the polymer units, such as the PEG units, in the hydrogel. In certain embodiments, the hydrogel is biodegraded within a period of about 1 month, or about 2 months, or about 3 months, or up to about 4 months, after administration. However, since during the degradation process in the aqueous environment, such as in the tear fluid within the canaliculus, the hydrogel gradually becomes softer and distorted, the insert may be cleared (washed out/disposed) through the nasolacrimal duct before it is completely biodegraded.

In embodiments of the present invention, the hydrogel and thus the insert remains in the canaliculus for a period of up to about 1 month, or up to about 2 months, or up to about 3 months, or up to about 4 months, after administration.

In certain embodiments of the invention, in the case the glucocorticoid is dexamethasone, the entire amount of dexamethasone may be released prior to the complete degradation of the hydrogel, and the insert may persist in the canaliculus thereafter, for a period of altogether up to about 1 month after administration, or up to about 2 months after administration, or up to about 3 months, or up to about 4 months, after administration. In certain other embodiments, the hydrogel may be fully biodegraded when the glucocorticoid, such as dexamethasone, has not yet been completely released from the insert. In other embodiments, the insert may be fully degraded following at least about 90%, or at least about 92%, or at least about 95%, or at least about 97% release of the glucocorticoid.

In certain embodiments, in vitro release tests may be used to compare different inserts (e.g. of different production batches, of different composition, and of different dosage strength etc.) with each other, for example for the purpose of quality control or other qualitative assessments. The in vitro-release of a glucocorticoid from the inserts of the invention can be determined by various methods, such as under non-sink simulated physiological conditions in PBS (phosphate-buffered saline, pH 7.4) at 37° C., with daily replacement of PBS in a volume comparable to the tear fluid in the human eye.

### Specific Insert Containing a Dose of about 200 μg Dexamethasone

In one particular embodiment, the present invention relates to a sustained release biodegradable intracanalicular insert containing dexamethasone in an amount in the range from about 160 μg to about 250 μg, or from about 180 μg to about 220 μg, or particularly in an amount of about 200 μg, wherein the dexamethasone is dispersed in a hydrogel. This insert in the dry state has a cylindrical or an essentially cylindrical shape with a diameter in the range of about 0.41 mm to about 0.49 mm and a length in the range of about 2.14 mm to about 2.36 mm. In the hydrated state (hydration in vivo in the canaliculus or in vitro, wherein hydration in vitro is measured in phosphate-buffered saline at a pH of 7.4 at 37° C. after 24 hours, which is considered equilibrium) this insert has a diameter in the range of about 1.35 mm to about 1.80 mm and a ratio of length to diameter of greater than 1, and may particularly have a length in the range of about 1.69 mm to about 1.87 mm.

In this insert, the hydrogel comprises a polymer network comprising crosslinked multi-arm polyethylene glycol units, particularly 4a 20 kPEG units, wherein the crosslinks between the PEG units include a group represented by the following formula

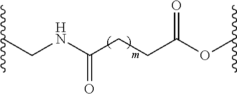

wherein m is 2. For making this insert, 4a 20 kPEG-SG units may be crosslinked by means of a crosslinking agent, such as trilysine acetate. In this embodiment, the insert may also contain a visualization agent, such as fluorescein, which is conjugated with the polymer network, such as with the trilysine acetate.

Also, in this embodiment the insert in a dry state is made up from about 40% to about 46% by weight dexamethasone and from about 45% to about 55% by weight polyethylene glycol units. The insert furthermore in its dry state may contain no more than about 1% by weight water.

The dexamethasone particles in this insert may have a d90 particle size of equal to or less than about 5 μm and/or a d98 particle size of less than about 10 μm as determined by laser diffraction.

This insert provides for a release of a therapeutically effective amount of dexamethasone over a period of up to about 7 days or more, such as up to about 14 days, up to about 21 days, or up to about 25 days, or up to about 1 month, after administration. In specific embodiments, this insert provides for a release of a therapeutically effective amount of dexamethasone over a period of up to about 14 days after administration (i.e., for about 2 weeks or up to about 2 weeks).

This insert may be used in the treatment of DED, including the acute treatment of DED or of episodic flares of DED.

### Specific Insert Containing a Dose of about 300 μg Dexamethasone

In another particular embodiment, the present invention relates to a sustained release biodegradable intracanalicular insert containing dexamethasone in an amount in the range from about 240 μg to about 375 μg, or from about 270 μg to about 330 μg, or particularly in an amount of about 300 μg, wherein the dexamethasone is dispersed in a hydrogel. This insert in the dry state has a cylindrical or an essentially cylindrical shape with a diameter in the range of about 0.44 mm to about 0.55 mm and a length in the range of about 2.14 mm to about 2.36 mm. In the hydrated state (hydration in vivo in the canaliculus or in vitro, wherein hydration in vitro is measured in phosphate-buffered saline at a pH of 7.4 at 37° C. after 24 hours, which is considered equilibrium) this insert has a diameter in the range of about 1.35 mm to about 1.80 mm and a ratio of length to diameter of greater than 1, and may particularly have a length in the range of about 1.64 mm to about 2.0 mm.

In this insert, the hydrogel comprises a polymer network comprising crosslinked multi-arm polyethylene glycol units, particularly 4a 20 kPEG units, wherein the crosslinks between the PEG units include a group represented by the following formula

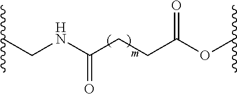

wherein m is 2. For making this insert, 4a 20 kPEG-SG units may be crosslinked by means of a crosslinking agent, such as trilysine acetate. In this embodiment, the insert may also contain a visualization agent, such as fluorescein, which is conjugated with the polymer network, such as with the trilysine acetate.

Also, in this embodiment the insert in a dry state is made up from about 50% to about 56% by weight dexamethasone and from about 36% to about 46% by weight polyethylene glycol units. The insert furthermore in its dry state may contain no more than about 1% by weight water.

The dexamethasone particles in this insert may have a d90 particle size of equal to or less than about 5 μm and/or a d98 particle size of less than about 10 μm as determined by laser diffraction.

This insert provides for a release of a therapeutically effective amount of dexamethasone over a period of up to about 14 days or more, such as up to about 21 days, or up to about 25 days, or up to about 1 month, after administration. In specific embodiments, this insert provides for a release of a therapeutically effective amount of dexamethasone over a period of up to about 21 days after administration (i.e., for about 3 weeks or up to about 3 weeks).

This insert may be used in the treatment of DED, including the acute treatment of DED or of episodic flares of DED.

### II. Manufacture of the Insert

In certain embodiments, the present invention also relates to a method of manufacturing a sustained release biodegradable intracanalicular insert as disclosed herein, comprising a hydrogel and a glucocorticoid, such as dexamethasone.

In certain embodiments the method of manufacturing according to the present invention comprises the steps of forming a hydrogel comprising a polymer network (e.g., comprising PEG units) and glucocorticoid particles dispersed in the hydrogel, shaping or casting the hydrogel and drying the hydrogel. In one embodiment the glucocorticoid, such as dexamethasone, may be used in micronized form as disclosed herein for preparing the insert. In another embodiment, the glucocorticoid, such as dexamethasone, may be used in non-micronized form for preparing the insert.

Suitable precursors for forming the hydrogel of certain embodiments of the invention are as disclosed above in the section relating to the insert itself. In certain specific embodiments, the hydrogel is made of a polymer network comprising crosslinked polyethylene glycol units as disclosed herein. The polyethylene glycol (PEG) units in particular embodiments are multi-arm, such as 4-arm, PEG units having an average molecular weight from about 2,000 to about 100,000 Daltons, or from about 10,000 to about 60,000 Daltons, or from about 15,000 to about 50,000 Daltons, or of about 20,000 Daltons. Suitable PEG precursors having reactive groups such as electrophilic groups as disclosed herein are crosslinked to form the polymer network. Crosslinking may be performed by means of a crosslinking agent that is either a low molecular compound or another polymeric compound, including another PEG precursor, having reactive groups such as nucleophilic groups as also disclosed herein. In certain embodiments, a PEG precursor with electrophilic end groups is reacted with a crosslinking agent (a low-molecular compound, or another PEG precursor) with nucleophilic end groups to form the polymer network.

In specific embodiments, the method of manufacturing the insert of the present invention comprises mixing and reacting an electrophilic group-containing multi-arm polyethylene glycol, such as 4a 20 kPEG-SG, with a nucleophilic group-containing crosslinking agent, such as trilysine acetate, in a buffered solution in the presence of dexamethasone particles, and allowing the mixture to gel. In certain embodiments, the molar ratio of the electrophilic groups in the PEG precursor to the nucleophilic groups in the crosslinking agent is about 1:1, but may also be in a range from about 2:1 to about 1:2.

In certain embodiments, a visualization agent as disclosed herein is included in the mixture forming the hydrogel so that the insert can be visualized once it has been administered into the canaliculus. For example, the visualization agent may be a fluorophore, such as fluorescein or a molecule comprising a fluorescein moiety, or another visualization agent as disclosed above. In certain embodiments, the visualization agent may be firmly conjugated with one or more components of the polymer network so that it remains in the insert at all times until the insert is biodegraded.

The visualization agent may for example be conjugated with either the polymer, such as the PEG, precursor, or the (polymeric or low molecular weight) crosslinking agent. In specific embodiments, the visualization agent is fluorescein and is conjugated to the trilysine acetate crosslinking agent prior to reacting the crosslinking agent with the PEG precursor, as illustrated in Example 1. For example, in the case of fluorescein, NHS-fluorescein (N-hydroxysuccinimidyl-fluorescein) may be reacted with trilysine acetate, and completion of the formation of the trilysine-fluorescein conjugate may be monitored (e.g. by means of RP-HPLC with UV-detection). This conjugate may then be used further to crosslink the polymeric precursor(s), such as the 4a 20 kPEG-SG.

In certain particular embodiments, during the manufacture of an insert of the present invention a (optionally buffered) mixture/suspension of the glucocorticoid and the PEG precursor(s), such as the dexamethasone and the 4a 20 kPEG-SG, in water is prepared. This glucocorticoid/PEG precursor mixture is then combined with a (optionally buffered) solution containing the crosslinking agent and the visualization agent conjugated thereto, such as the lysine acetate/fluorescein conjugate. The resulting combined mixture thus contains the glucocorticoid, the polymer precursor(s), the crosslinking agent, the visualization agent and (optionally) buffer. This is illustrated exemplarily in Example 1.

In certain embodiments, once the mixture of the electrophilic group-containing polymer precursor, the nucleophilic group-containing crosslinking agent, the glucocorticoid, such as dexamethasone, optionally the visualization agent (optionally conjugated to e.g. the crosslinking agent), and optionally buffer has been prepared (i.e., after these components have been combined), the resulting mixture is cast into a suitable mold or tubing prior to complete gelling in order to provide e.g. a hydrogel strand, and ultimately the desired final shape of the hydrogel. The mixture is then allowed to gel. The resulting hydrogel is then dried.

In case the final shape of the insert is cylindrical or is essentially cylindrical, a hydrogel strand is prepared by casting the hydrogel precursor mixture comprising the glucocorticoid particles into a fine diameter tubing, such as a polyurethane (PU) tubing. Different geometries and diameters of the tubing may be used, depending on the desired final cross-sectional geometry of the hydrogel strand and thus the final insert, its initial diameter (which may still be decreased by means of stretching), and depending also on the ability of the reactive mixture to uniformly fill the tubing and to be removed from the tubing after drying. Thus, the inside of the tubing may have a round geometry or a non-round geometry, such as an oval (or other) geometry.

In certain embodiments, after the hydrogel strand has been formed and has been left to cure and to complete the gelling process within the tubing, the hydrogel strand may be longitudinally stretched in the wet or dry state as disclosed herein. The stretching may result in a dimensional change of the insert upon hydration, e.g. after it has been placed into the canaliculus. In particular embodiments, the hydrogel strand is stretched prior to (complete) drying by a stretching factor in a range of about 1 to about 3, or of about 1.5 to about 3, or of about 2.2 to about 2.8, or of about 2.5 to about 2.6. In certain embodiments, the stretching may be performed when the hydrogel strand is still in the tubing. Alternatively, the hydrogel strand may be removed from the tubing prior to being stretched. In the case dry stretching is performed in certain embodiments of the invention, the hydrogel strand is first dried and then stretched (when still inside of the tubing, or after having been removed from the tubing). When wet stretching is performed in certain embodiments of the invention, the hydrogel is stretched in a wet state (i.e., before it has dried completely) and then left to dry under tension. Optionally, heat may be applied upon stretching.

After stretching and drying the hydrogel strand may be removed from the tubing and cut into segments of a desired length, such as disclosed herein, to produce the final insert (if cut within the tubing, the cut segments are removed from the tubing after cutting). A particularly desired length for the purposes of the present invention is for example a length of equal to or less than about 2.75 mm, or equal to or less than about 2.5 mm, such as a length in the range of about 2.0 mm to about 2.5 mm, or of about 2.14 mm to about 2.36 mm, for example a length of about 2.25 mm.

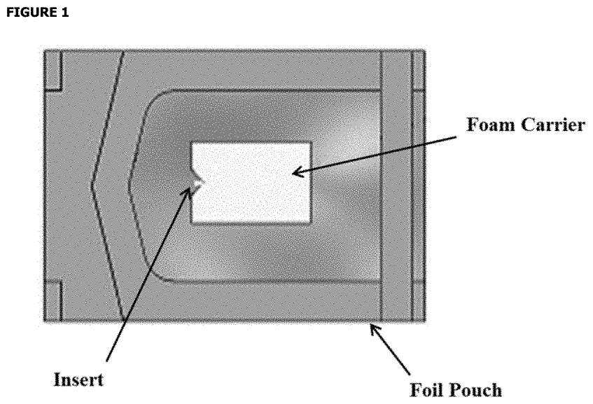

After cutting, the inserts may then be packaged into a packaging that keeps out moisture, such as a sealed foil pouch. The inserts may be fixated to a mount or support to keep them in place and to avoid damage to the insert, and also to facilitate removing the insert from the packaging and gripping/holding the insert for administration to a patient. For example, an insert of the present invention may be fixated into the opening of a foam carrier, with a portion of the insert protruding for easy removal and gripping (as illustrated in FIG. 1). The insert may be removed from the foam carrier by means of forceps and then immediately inserted into the canaliculus of a patient.

A particular embodiment of a manufacturing process according to the invention is disclosed in detail in Example 1.

### III. Therapy

In one aspect, the present invention relates to a method of treating dry eye disease (DED) in a patient in need thereof, the method comprising administering to the patient a sustained release biodegradable ocular (such as intracanalicular) insert as disclosed herein.

In another aspect the present invention also relates to a method of treating episodic flares of DED in a patient in need thereof, the method comprising administering to the patient a sustained release biodegradable ocular (such as intracanalicular) insert comprising a hydrogel and a glucocorticoid, wherein glucocorticoid particles are dispersed within the hydrogel.

The patient to be treated in accordance with the invention may be a human or animal subject in need of DED therapy, including acute DED therapy. In certain embodiments, the patient may be a subject in need of acute treatment of an episodic flare of DED.

In certain alternative embodiments, the treatment of DED may be a long (or longer) term treatment of DED.

In one embodiment, the present invention also relates to a sustained release biodegradable ocular (such as intracanalicular) insert as disclosed herein for use in treating DED in a patient in need thereof.

In one embodiment, the present invention also relates to the use of a sustained release biodegradable ocular (such as intracanalicular) insert as disclosed herein for the manufacture of a medicament for treating DED in a patient in need thereof.

In certain embodiments, the treatment of DED is an acute, short-term treatment of DED (also herein referred to as a treatment of episodic flares of DED). The period for acute treatment of episodic flares of DED according to the present invention may be relatively short as compared to a continuous longer-term (i.e., chronic) treatment of DED, and may in certain embodiments be up to about 1 month or about 1 month, or up to about 25 days or about 25 days, or up to about 21 days or about 21 days (i.e., up to about 3 weeks or about 3 weeks), or up to about 14 days or about 14 days (i.e., up to about 2 weeks or about 2 weeks), or up to about 7 days or about 7 days (i.e., up to about 1 week or about 1 week). A longer-term (chronic) treatment of DED may last longer than about 1 month, such as several months or even longer.

In certain embodiments, the sustained release biodegradable intracanalicular insert administered to the patient comprises dexamethasone.

In certain embodiments, the sustained release biodegradable intracanalicular insert administered to the patient comprises equal to or less than about 375 μg, or equal to or less than about 350 μg dexamethasone or an equivalent dose of another glucocorticoid. The equivalent dose of another glucocorticoid can be determined as disclosed herein. In embodiments of the invention, the glucocorticoid, such as dexamethasone, is present in the insert as particles dispersed in a hydrogel formed of a polymer network as disclosed herein.

In certain embodiments, the sustained release biodegradable intracanalicular insert administered to the patient comprises a dose in the range of about 100 μg to about 350 μg dexamethasone, or in the range of about 150 μg to about 320 μg dexamethasone, or an equivalent dose of another glucocorticoid.

In certain embodiments, the sustained release biodegradable intracanalicular insert administered to the patient comprises from about 160 μg to about 250 μg dexamethasone, or from about 180 μg to about 220 μg dexamethasone, or about 200 μg dexamethasone.

In certain other embodiments, the sustained release biodegradable intracanalicular insert administered to the patient comprises from about 240 μg to about 375 μg dexamethasone, or from about 270 μg to about 330 μg dexamethasone, or about 300 μg dexamethasone.

The insert may contain additional ingredients as disclosed herein.

Administration of the insert according to the invention is performed through the opening of the punctum into the inferior and/or superior canaliculus.

In certain embodiments, upon hydration in vivo after administration in the canaliculus the sustained release biodegradable intracanalicular insert administered to the patient increases in diameter and may decrease in length as disclosed herein.

In certain embodiments, the sustained release biodegradable intracanalicular insert administered to the patient has a length of equal to or less than about 2.75 mm, or equal to or less than about 2.5 mm in a dry state. In certain embodiments, the sustained release biodegradable intracanalicular insert administered to the patient has a diameter of less than about 1 mm, or a diameter of less than about 0.75 mm in the dry state. An insert that has such a diameter in the dry state may be administered easily through the punctum of the eye. In particular ones of these embodiments, the glucocorticoid is dexamethasone.

In certain particular embodiments, the sustained release biodegradable intracanalicular insert administered to the patient in a dry state has a diameter in the range of about 0.41 mm to about 0.55 mm and a length in the range of about 2.14 mm to about 2.36 mm. In certain embodiments, the sustained release biodegradable intracanalicular insert administered to the patient has a diameter of about 0.5 mm and a length of about 2.25 mm in a dry state. In particular embodiments, the glucocorticoid in this insert is dexamethasone and is present in the insert in an amount of about 200 μg or about 300 μg (including the variances as disclosed herein)

In certain other particular embodiments, the sustained release biodegradable intracanalicular insert administered to the patient has a diameter in the range of about 1.35 mm to about 1.80 mm and a ratio of length to diameter of greater than 1 when it is in the hydrated state. In certain embodiments, the sustained release biodegradable intracanalicular insert administered to the patient has a diameter in the range of about 1.40 mm to about 1.60 mm, such as about 1.5 mm and a length in the range of about 1.70 mm to about 2.0 mm when it is in the hydrated state. In particular embodiments, the glucocorticoid in this insert is dexamethasone and is present in the insert in an amount of about 200 μg or about 300 μg (including the variances as disclosed herein).

In certain embodiments, the method provides for the release of a glucocorticoid, such as dexamethasone, for an extended period of time (“extended” as opposed to known immediate release ophthalmic dosage forms) of about 6 hours or longer, such as for a period of about 12 hours or longer after administration. In certain embodiments, the extended period of time is one or more weeks after administration.

In certain embodiments, the method provides for (acute, short-term) treatment of episodic flares of DED (“short” as opposed to a long-term treatment of DED as defined herein), providing a treatment with glucocorticoid such as dexamethasone for a period of up to about 7 days, or up to about 14 days, or up to about 21 days, or up to about 25 days, or up to about 1 month, after administration.

In certain embodiments of the method of the present invention, the sustained release biodegradable intracanalicular insert administered to the patient contains about 200 μg dexamethasone (including the variances as disclosed herein) and releases dexamethasone for a period of up to about 14 days after administration, such that the period of treatment provided by the dexamethasone release from the insert is up to about 14 days after administration. In certain embodiments, this treatment period may also be longer than about 14 days after administration.

In certain embodiments of the method of the present invention, the sustained release biodegradable intracanalicular insert administered to the patient contains about 300 μg dexamethasone (including the variances as disclosed herein) and releases dexamethasone for a period of up to about 21 days after administration, such that the period of treatment provided by the dexamethasone release from the insert is up to about 21 days after administration. In certain embodiments, this treatment period may also be longer than about 21 days after administration.

In certain embodiments of the method of the present invention, the sustained release biodegradable intracanalicular insert administered to the patient releases on average from about 15 to about 25 μg dexamethasone per day for an extended period of time, such for as up to about 7 days, or up to about 14 days, or up to about 21 days, or up to about 25 days, or up to about 1 month, after administration.

In certain of these embodiments, the sustained release biodegradable intracanalicular insert administered to the patient contains about 200 μg dexamethasone and releases on average from about 15 μg to about 25 μg dexamethasone per day for a period of up to about 7 days after administration. Thereafter, the insert may still release dexamethasone, but may release dexamethasone at a lower rate (i.e., may release a lower amount of dexamethasone per day), also referred to herein as “tapered release”.

In certain other embodiments, the sustained release biodegradable intracanalicular insert administered to the patient contains about 300 μg dexamethasone and releases on average from about 15 μg to about 25 μg dexamethasone per day for a period of up to about 11, or up to about 14 days after administration. Again, thereafter, the insert may still release dexamethasone, but may release dexamethasone at a lower rate (i.e., may release a lower amount of dexamethasone per day), also referred to herein as “tapered release”.

In one embodiment of the method of the present invention, the sustained release biodegradable intracanalicular insert administered to the patient in the method of treatment of DED, including episodic flares of DED, according to the present invention comprises a hydrogel and dexamethasone particles dispersed within the hydrogel, wherein the insert contains from about 160 μg to about 250 μg or from about 180 μg to about 220 μg or about 200 μg dexamethasone, is cylindrical or essentially cylindrical and has in a dry state a diameter in the range of about 0.41 mm to about 0.49 mm and a length in the range of about 2.14 mm to about 2.36 mm, and in a hydrated state a diameter from about 1.35 mm to about 1.80 mm and a ratio of length to diameter of greater than 1, and releases dexamethasone for a period of up to about 14 days, or up to about 21 days after administration. In this embodiment, the hydrogel comprises a polymer network, wherein the polymer network comprises 4a 20 kPEG units and is formed by reacting 4a 20 kPEG-SG precursor with trilysine or a trilysine acetate as crosslinking agent. In this embodiment, the insert further comprises a visualization agent such as fluorescein. Also, in this embodiment the insert in a dry state is made up from about 40% to about 46% by weight dexamethasone and from about 45% to about 55% by weight polyethylene glycol units. The insert furthermore in its dry state may contain no more than about 1% by weight water. The treatment period with this insert may be up to or about 14 days (i.e., about 2 weeks).

In another embodiment of the method of the present invention, the sustained release biodegradable intracanalicular insert administered to the patient in the method of treatment of DED, including episodic flares of DED, according to the present invention comprises a hydrogel and dexamethasone particles dispersed within the hydrogel, wherein the insert contains from about 240 μg to about 375 μg or from about 270 μg to about 330 μg or about 300 μg dexamethasone, is cylindrical or essentially cylindrical and has in a dry state a diameter in the range of about 0.44 mm to about 0.55 mm and a length in the range of about 2.14 mm to about 2.36 mm, and in a hydrated state a diameter in the range of about 1.35 mm to about 1.80 mm and a ratio of length to diameter of greater than 1, and releases dexamethasone for a period of up to about 21 days, or up to about 25 days, or up to about 1 month, after administration. In this embodiment, the hydrogel comprises a polymer network, wherein the polymer network comprises 4a 20 kPEG units and is formed by reacting 4a 20 kPEG-SG precursor with trilysine acetate or derivative as crosslinking agent. In this embodiment, the insert further comprises a visualization agent such as fluorescein. Also, in this embodiment the insert in a dry state is made up from about 50% to about 56% by weight dexamethasone and from about 36% to about 46% by weight polyethylene glycol units. The insert furthermore in its dry state may contain no more than about 1% by weight water. The treatment period with this insert may be up to or about 21 days (i.e., about 3 weeks).

In a yet further embodiment of the method of the present invention, in case the sustained release biodegradable intracanalicular insert administered to the patient is defined by a dose of equal to or less than about 375 μg dexamethasone (as disclosed herein above) or an equivalent dose of another glucocorticoid contained in the insert, and/or by a length of equal to or less than about 2.75 mm (as disclosed herein below), the insert may release a therapeutically effective amount of a glucocorticoid for a period of longer than about 25 days after administration, such as up to about 1 month, or even longer, after administration.

As only one single insert (or in certain exceptional embodiments several inserts at the same time) needs to be administered to the patient to achieve prolonged delivery times, such as for a period of up to about 7 days, or up to about 14 days, or up to about 21 days, or up to about 25 days, or up to about 1 month after administration, or even longer in certain embodiments, patient compliance is increased as compared to the use of eye drops that have to be administered daily or even several times per day.

In certain embodiments, the insert is administered unilaterally or is administered bilaterally through the lower punctum to the inferior canaliculus. In other embodiments, the insert is administered unilaterally or is administered bilaterally through the upper punctum to the superior canaliculus. In certain embodiments, the insert is administered both through the lower punctum to the inferior canaliculus and through the upper punctum to the superior canaliculus. The particular administration per eye can be independent of the other.

In certain embodiments, the insert is administered to the inferior vertical canaliculus and/or the superior vertical canaliculus.

In certain embodiments, the sustained release biodegradable intracanalicular insert comprises a visualization agent such as fluorescein to enable quick and noninvasive visualization of the insert when placed inside the canaliculus. In case the visualization agent is fluorescein, the insert may be visualized by illuminating with a blue light source and using a yellow filter.

In certain embodiments, the glucocorticoid such as dexamethasone is delivered from the insert to the ocular surface through the tear film as the glucocorticoid dissolves in the tear film when released from the insert. The glucocorticoid is released primarily from the proximal end of the insert at the interface between the hydrogel and the tear fluid (as exemplarily shown in FIG. 6). The sustained glucocorticoid release rate is controlled by glucocorticoid solubility in the hydrogel matrix and the tear fluid. In certain embodiments, the glucocorticoid is dexamethasone, which has a low solubility in aqueous medium as disclosed herein.

In certain embodiments, the insert remains in the canaliculus after complete depletion of the glucocorticoid such as dexamethasone from the insert until the hydrogel has biodegraded and/or is disposed (washed out/cleared) through the nasolacrimal duct. As the hydrogel matrix of the insert is formulated to biodegrade e.g. via ester hydrolysis in the aqueous environment of the tear fluid in the canaliculus, the insert softens and liquefies over time and is cleared through the nasolacrimal duct without the need for removal. Unpleasant removal may thus be avoided. However, in case an insert should be removed e.g. because of a potential allergic reaction or other circumstances which require removal of the insert, such as an unpleasant foreign body sensation felt by a patient, or because treatment should be terminated for another reason, the insert may be expelled from the canaliculus e.g. manually.

In certain embodiments, the insert remains in the canaliculus for up to about 1 month, or up to about 2 months, or up to about 3 months, or up to about 4 months after administration.

In certain embodiments the systemic concentration of glucocorticoid such as dexamethasone after administration of the insert of the present invention is very low, such as below quantifiable amounts. This significantly reduces the risk of drug-to-drug interactions or systemic toxicity, which can be beneficial e.g. in older patients who are frequently suffering from ocular diseases and are additionally taking other medications.

In certain embodiments, the sustained release biodegradable intracanalicular inserts of the present invention are administered to a patient for the treatment of signs and symptoms of dry eye disease (DED), in particular for the acute treatment of DED, for instance, upon episodic flares. In certain embodiments, the inserts of the present invention combine the effect of inflammation suppression due to glucocorticoid release such as dexamethasone release with benefits derived from lacrimal occlusion. These combined effects may provide for an improved treatment of DED.

In certain embodiments, the treatment of DED by means of the sustained release biodegradable intracanalicular insert of the present invention can be combined with or followed up by another treatment of DED. In certain embodiments, the treatment of DED by means of the sustained release biodegradable intracanalicular insert is combined with or followed up by chronic treatment of DED, for instance by chronic treatment with cyclosporine, lifitegrast or tacrolimus. In certain other embodiments, the treatment of DED by means of the sustained release biodegradable intracanalicular insert is combined with or followed up by treatment with ophthalmic drops, such as artificial tears.

In certain embodiments, as the insert of the present invention is located in the canaliculus and therefore not on the surface of the eye, and only one single administration is required to provide for the release of a glucocorticoid for an extended period of time as disclosed herein, the insert does not interfere or substantially interfere with contact lenses and may therefore be particularly suitable and convenient for patients wearing contact lenses.

A patient treated with an insert of the present invention may be any person in need of treatment. The patient may be male or female. In certain embodiments, the patient is female. In certain embodiments, the patient is over 50 years of age, or over 60 years of age, or over 70 years of age, or over 80 years of age. In certain embodiments, the patient may have had laser surgery of the eye, such as photorefractive keratectomy (PRK) or Laser-in-situ-Keratomileusis (LASIK) or any particular varieties thereof.

In certain embodiments, the patient treated with an insert of the present invention is on artificial tears and/or other palliative treatments and experiences episodic DED flares.

In certain embodiments, the patient requires induction therapy while initiating treatment of DED. Pre-treatment with the sustained release biodegradable intracanalicular inserts according to the present invention before initiating long-term therapy for DED can lead to a faster resolution of signs and symptoms of DED and to a reduction in side effects such as burning or stinging caused by active ingredients for DED treatment such as cyclosporine. The pre-treatment period with the insert according to the present invention may last, e.g., for up to about 7 days, or up to about 14 days, or up to about 21 days, or in specific embodiments up to about 1 month prior to initiating a longer-term therapy.

In certain embodiments, the patient treated with an insert of the present invention is on chronic DED treatment with for instance cyclosporine or lifitegrast and experiences episodic flares of DED which may be treated with an insert according to the present invention.

In certain embodiment, the patient treated with an insert of the present invention requires treatment of DED before cataract and refractive surgery to improve outcomes/satisfaction of such surgery.

In certain embodiments, the patient treated with an insert of the present invention requires short-term treatment of signs and symptoms of DED after cataract or refractive surgery.

In certain embodiments, the treatment of DED with the inserts disclosed herein are combined with the application of artificial tears and/or ophthalmic drops and/or nasal neurostimulatory devices. The co-administered artificial tears and/or ophthalmic drops and/or nasal neurostimulatory devices may comprise anti-infective ingredients. In certain embodiments, the co-administered ophthalmic drops may comprise other glucocorticoids than the glucocorticoid of the administered insert (such as dexamethasone). In certain embodiments, the co-administered ophthalmic drops may comprise the same glucocorticoid as the glucocorticoid contained in the administered insert (such as dexamethasone). Suitable pharmacological products for co-administration include Restasis® and Cequa® (ophthalmic drops comprising cyclosporine), Xiidra® (ophthalmic drops comprising the lymphocyte function-associated antigen 1 (LFA-1) antagonist lifitegrast), and TrueTear® (nasal neurostimulatory device to temporarily increase tear production). In certain embodiments that may be combined with any of the embodiments of this paragraph, the treatment of DED by means of the sustained release biodegradable intracanalicular insert is combined with eyelid exfoliation at the lash line (BlephEx®), eyelid thermal pulsation (iLux®), and eyelid warming and stimulation (LipiFlow®).

In certain embodiments of the present invention, a further sustained release biodegradable intracanalicular insert is administered into the canaliculus through the ocular punctum while the first sustained release biodegradable intracanalicular insert is still retained in the canaliculus (which procedure is referred to as “insert stacking” or short “stacking”), either while the first insert still releases glucocorticoid, or after the first insert has been completely depleted of glucocorticoid, or after the first insert has been partially depleted of glucocorticoid by at least about 70%, or at least about 80%, or at least about 90% and/or the first insert releases a lower amount of glucocorticoid than initially after its administration.

In certain embodiments, insert stacking enables prolonged treatment with a glucocorticoid such as dexamethasone. In certain embodiments, insert stacking thus provides for a release of a therapeutically effective amount of glucocorticoid for a total period of up to about 14 days, or up to about 28 days, or up to about 42 days, or up to about 50 days, or up to about 2 months after administration of the first insert.

### IV. Kit

In certain embodiments, the present invention is further directed to a kit comprising one or more insert(s) as disclosed herein or manufactured in accordance with the methods as disclosed herein.

In certain specific embodiments, the kit comprises one or more sustained release biodegradable intracanalicular insert(s), wherein each insert contains from about 160 μg to about 250 μg or from about 180 μg to about 220 μg or about 200 μg dexamethasone and has in a dry state a diameter in the range of about 0.41 mm to about 0.49 mm and a length in the range of about 2.14 mm to about 2.36 mm, and has in the hydrated state a diameter in the range of about 1.35 mm to about 1.80 mm and a ratio of length to diameter of greater than 1, and wherein each insert provides for a release of dexamethasone for a period of up to about 14 days, or up to about 21 days, after administration.

In certain other specific embodiments, the kit comprises one or more sustained release biodegradable intracanalicular insert(s), wherein each insert contains from about 240 μg to about 375 μg or from about 270 μg to about 330 μg or about 300 μg dexamethasone and has in a dry state a diameter in the range of about 0.44 mm to about 0.55 mm and a length in the range of about 2.14 mm to about 2.36 mm, and has in the hydrated state a diameter in the range of about 1.35 mm to about 1.80 mm and a ratio of length to diameter of greater than 1, and wherein each insert provides for a release of dexamethasone for a period of up to about 21 days, or up to about 1 month, after administration.

In certain embodiments, the kit further comprises instructions for using the one or more sustained release biodegradable intracanalicular insert(s). The instructions for using the one or more sustained release biodegradable intracanalicular insert(s) may be in the form of an operation manual for the physician who is administering the insert(s). The kit may further comprise a package insert with product-related information.

In certain embodiments, the kit may further comprise one or more means for administration of the one or more sustained release biodegradable intracanalicular insert(s). The means for administration may be for example one or more suitable tweezer(s) or forceps, either for one time use or for repeated use. For instance, suitable forceps are blunt (non-toothed). The means for administration may also be an injection device such as a syringe or applicator system.

In certain embodiments, the kit may further comprise an ophthalmic dilator to dilate the punctum prior to the administration of the one or more sustained release biodegradable intracanalicular insert(s) and thereby facilitate insertion of the insert(s) through the punctum into the canaliculus. A dilator may also be combined/integrated with forceps or an applicator, such that e.g. one end of the device is a dilator, and the other end of the device is suitable to administer the insert. Alternatively, the kit may also contain a modified applicator that e.g. has a tapered tip that may be used for both dilation and insertion.

In certain embodiments, the one or more sustained release biodegradable intracanalicular insert(s) are individually packaged for a single administration. In certain embodiments, the one or more sustained release biodegradable intracanalicular insert(s) are individually packaged for a single administration by fixating each insert in foam carrier, which is sealed in a foil pouch. The foam carrier may have e.g. a V-notch or a circular incision with an opening at the bottom of the V-notch to hold the insert (see, for instance, also FIG. 1).

If two or more sustained release biodegradable intracanalicular inserts are contained in the kit, these inserts may be identical or different, and may contain identical or different doses of the glucocorticoid such as dexamethasone.

In certain embodiments, the compositions and methods disclosed herein release an effective amount of glucocorticoid to treat bulbar conjunctival hyperemia.

In certain embodiments, the compositions and methods disclosed herein provide improvement in bulbar conjunctival hyperemia from baseline to day 15.

In certain embodiments, the compositions and methods disclosed herein provide an improvement in bulbar conjunctival hyperemia from baseline to day 15 using a central reading photographic assessment.

In certain embodiments, the compositions and methods disclosed herein provide an improvement in bulbar conjunctival hyperemia from baseline to day 15 using a central reading photographic assessment in a modified ITT population.

In certain embodiments, the compositions and methods disclosed herein provide an improved bulbar conjunctival hyperemia from baseline to day 15 compared to vehicle hydrogel using a central reading photographic assessment in a modified ITT population.

In certain embodiments, the compositions and methods disclosed herein provide a change in baseline using the CCLRU Grading Scale of −0.1 to −0.8.

In certain embodiments, the compositions and methods disclosed herein provide a change in baseline using the CCLRU Grading Scale of −0.25 to −0.8.

In certain embodiments, the compositions and methods disclosed herein provide a change in baseline using the CCLRU Grading Scale of −0.3 to −0.5 or about −0.3 or about −0.4 or about −0.05.

In certain embodiments, the compositions and methods disclosed herein provide a retention after intracanalicular administration of at least 15 day, at least 20 days, at least 25 days or at least 30 days.

In certain embodiments, the compositions and methods disclosed herein provide an improvement in Severity of Eye Dryness Score (visual analogue scale [VAS]) CFB at 15 days.

In certain embodiments, the compositions and methods disclosed herein provide an improvement in Frequency of Eye Dryness (VAS) CFB at 15 days.

In certain embodiments, the compositions and methods disclosed herein provide an improvement in Severity of Eye Dryness Score (VAS), CFB and absolute values post-baseline.

In certain embodiments, the compositions and methods disclosed herein provide an improvement in Frequency Eye Dryness Score (VAS), CFB and absolute values post baseline.

In certain embodiments, the compositions and methods disclosed herein provide an improvement in Investigator assessment of bulbar conjunctival hyperemia CFB post baseline.

In certain embodiments, the compositions and methods disclosed herein provide an improvement in Total Corneal Fluorescein Staining (tCFS) [National Eye Institute (NEI) scale], CFB and absolute values post baseline.

In certain embodiments, the compositions and methods disclosed herein provide an improvement in CFS sub-regions using NEI scale, CFB and absolute values post baseline.

In certain embodiments, the compositions and methods disclosed herein provide an improvement in Ocular Surface Disease Index questionnaire (OSDI©) questionnaire, CFB and absolute values (total score, each of the three domains, and individual questions) post baseline.

In certain embodiments, the compositions and methods disclosed herein provide an improvement in SPEED questionnaire (overall score and individual questions), CFB post baseline.

In certain embodiments, the compositions and methods disclosed herein provide a mean absolute value at day 15 of conjunctival hyperemia Total (Scale 0-12) of less than 5.2, less than 5, less than 4.8, less than 4.6, or less than 4.4.

In certain embodiments, the compositions and methods disclosed herein provide a conjunctival hyperemia change in baseline at day 15 of less than −0.15, less than −0.25, less than −0.35, less than −0.50, less than −0.65 or less than 0.75.

In certain embodiments, the compositions and methods disclosed herein provide a mean absolute value at day 15 of conjunctival hyperemia in nasal zone of less than 1.75, less than 1.7, less than 1.6, or less than 1.5.

In certain embodiments, the compositions and methods disclosed herein provide a conjunctival hyperemia in nasal zone change in baseline at day 15 of less than −0.2, less than −0.3, or less than 0.4.

In certain embodiments, the compositions and methods disclosed herein provide a mean absolute value at day 15 of conjunctival hyperemia in temporal zone of less than 1.8, less than 1.7, less than 1.6 or less than 1.5.

In certain embodiments, the compositions and methods disclosed herein provide a conjunctival hyperemia in temporal zone change in baseline at day 15 of less than −0, less than −0.1, or less than 0.2.

In certain embodiments, the compositions and methods disclosed herein provide a mean absolute value at day 15 of conjunctival hyperemia in frontal zone of less than 1.7, less than 1.6, less than 1.5, or less than 1.4.

In certain embodiments, the compositions and methods disclosed herein provide a conjunctival hyperemia in frontal zone change in baseline at day 15 of less than −0.1, less than −0.2, or less than 0.3.

In certain embodiments, the compositions and methods disclosed herein provide an occurrence of eye pruritus of less than 4%, less than 3%, less than 2% or less than 1% in a patient population.

In certain embodiments, the compositions and methods disclosed herein provide an occurrence of lacrimation increase of less than 15%, less than 10%, less than 6% or less than 4% in a patient population.

In certain embodiments, the compositions and methods disclosed herein provide an occurrence of IOP elevation of less than 8%, less than 6%, less than 5% or less than 4% in a patient population.

The studies herein used assessments that included the following:

### Schirmer's Tear Test

The Schirmer's test determines the amount of tears produced and works by capillary action, which allows the tear liquid to travel along the length of the paper test strip. The rate of travel along the test strip is proportional to the rate of tear production. The subject is asked to look up and the bent end of the test strip is applied such that it rests between the inferior palpebral conjunctiva of the lower eyelid and the bulbar conjunctiva of the eye. After five minutes, the patient is asked to open both eyes and look upward and the test strips are removed. The Schirmer's test score is determined by the length of the moistened area of the strips. Both eyes are tested at the same time. When anesthetized, only basal tear secretion is being measured.

A Schirmer's score of ≥10 mm wetting is considered normal, while a score of <5 mm indicates tear deficiency.

### Tear Film Break Up Time (TBUT) and Total Corneal Fluorescein Staining (tCFS)

The time required for the tear film to break up following a blink is called TBUT. It is a quantitative test for measurement of tear film stability. The normal time for tear film breakup is over 15 seconds. To assess TBUT, a fluorescein strip is moistened with saline and applied to the inferior cul-de-sac. After a couple of blinks, the tear film is examined using a broad-beam of slit lamp with a blue filter for the appearance of the first dry spots on the cornea.

TBUT values of less than 5-10 seconds indicate tear instability and are observed in patients with mild to moderate dry eye disease.

The total Corneal Fluorescein Staining (tCFS) value is measured to assess the condition of the cornea. Damages such as abrasions on the corneal surface, which may result e. g. from dry eyes, are made visible by a fluorescein dye staining.

To assess tCFS, a fluorescein strip is wetted with saline solution/eye wash, the subject is asked to look up and the moistened strip is applied to the inferior palpebral conjunctiva without touching the strip to the bulbar conjunctiva. Since TBUT is also assessed by applying fluorescein, if the tCFS measurement is done closely following the TBUT, then an additional application of fluorescein dye is not required. The subject is asked to blink several times to distribute the fluorescein dye, and after 2 to 3 minutes wait time, the cobalt blue illumination and the Wratten yellow filter is used to assess the corneal staining for each of the 5 regions of the cornea, central, inferior, nasal, temporal, and superior using the NEI (National Eye Institute) 0-3 scoring scale (0=No Staining, 1=Mild Staining, 2=Moderate Staining, 3=Severe Staining), wherein the CFS total score is the sum of the five areas (0 to 15).

The higher the tCFS score, the higher the damages on the corneal surface.

### Best Corrected Visual Acuity BCVA

Visual acuity testing should precede any examination requiring contact with the eye or instillation of study dyes. Log MAR visual acuity must be assessed using an Early Treatment Diabetic Retinopathy Study (ETDRS) or modified ETDRS chart, consisting of lines of five letters each, each line representing a 0.1 log unit of the minimum angle of resolution (log MAR) at a given test distance.

Visual acuity testing is performed using an Early Treatment Diabetic Retinopathy Study (ETDRS) or modified ETDRS chart with best correction using subject's own corrective lenses (spectacles only) or pinhole refraction. The ETDRS or modified ETDRS chart consists of lines of five letters each, each line representing a 0.1 log unit of the minimum angle of resolution (log MAR) at a given test distance.

Visual acuity (VA) is scored as a log MAR value, wherein the last line in which a letter is read correctly will be taken as the base log MAR reading, to which N×0.02 is added, with N being the total number of letters missed up to and included in the last line read. This total sum (base log MAR+N×0.02) represents the BCVA for that eye.

The lower the BCVA score, the better the visual acuity.

### Eye Dryness Score/Visual Analogue Scale VAS

In order to assess the eye dryness score the subject is asked to rate the severity and the frequency of symptom of eye dryness in percent by placing a vertical mark on a horizontal line (representing values from 0 to 100%) to indicate the level of eye discomfort that they are experiencing in both eyes currently and how often the eye dryness is experienced, wherein 0% corresponds to “no discomfort” and 100% corresponds to “maximal (the most) discomfort”.

### Ocular Surface Disease Index OSDI©

The OSDI allows to quickly assess the symptoms of ocular irritation in dry eye disease based on a 12-item questionnaire assessing dry eye symptoms and the effects it has on vision-related function in the past week of the subject's life (see e.g. by R. M. Schiffman et al. in Arch Ophthalmol. 2000; 118(5):615-621 hereby incorporated by reference).

The higher the final score, the greater the disability.

### Standard Patient Evaluation of Eye Dryness (SPEED) Evaluation

The SPEED questionnaire (see Korb and Blackie, Ocular Surgery News Europe Edition. 2012 hereby incorporated by reference) is another assessment for monitoring dry eye symptoms over time, with a score from 0 to 28 resulting from 8 items that assess frequency and severity of symptoms including dryness, grittiness, scratchiness, irritation, burning, watering, soreness, and eye fatigue.

Higher scores indicate greater disability.

### Hyperemia Grading

Assessment of bulbar conjunctiva is performed by central reading center using photographs at baseline and Day 15 Visit or by Investigators when using slit lamp biomicroscopy at baseline and each study follow up visit. Bulbar conjunctival hyperemia is then scored using CCLRU grading scale for nasal, temporal and frontal zones individually in each eye and captured in the source document as described below:

The frontal score: For observed conjunctiva in the straight ahead view (both nasal and temporal to cornea)

The nasal score: For observed conjunctiva when the eye is in lateral gaze (looking toward the ear)

The temporal score: For observed conjunctiva when the eye is in medial gaze (looking toward the nose)

CCLRU grading scale is explained below:

0=None: Vessels barely visible

1=Very Slight: Vessels visible but not dilated

2=Slight: Vessels visible and dilated

3=Moderate: Vessels visible, dilated, diffuse, bright red

4=Severe: Vessels visible, dilated, diffuse, deep red

In addition to the disclosure above, disclosed herein are also the following other Embodiments:

### First List of Embodiments

1. A method for treating dry eye in a subject in need thereof, comprising administering to said subject a biodegradable ocular hydrogel composition comprising an effective amount of a corticosteroid for a period of about 12 hours or longer.

2. The method of Embodiment I, wherein the polymer network comprises a plurality of polyethylene glycol (PEG) units.

3. The method of Embodiment 1 or 2, wherein the polymer network comprises a plurality of multi-arm PEG units having from 2 to 10 arms.

4. The method of any one of Embodiments 1 to 3, wherein the polymer network comprises a plurality of multi-arm PEG units having from 4 to 10 arms.

5. The method of any one of Embodiments 1 to 4, wherein the polymer network comprises a plurality of multi-arm PEG units having from 4 to 8 arms.

6. The method of any one of Embodiments 1 to 5, wherein the polymer network comprises a plurality of multi-arm PEG units having 8 arms.

7. The method of any one of Embodiments 1 to 5, wherein the polymer network comprises a plurality of multi-arm PEG units having 4 arms.

8. The method of any one of Embodiments 1 to 5, wherein the polymer network comprises a plurality of PEG units having the formula:

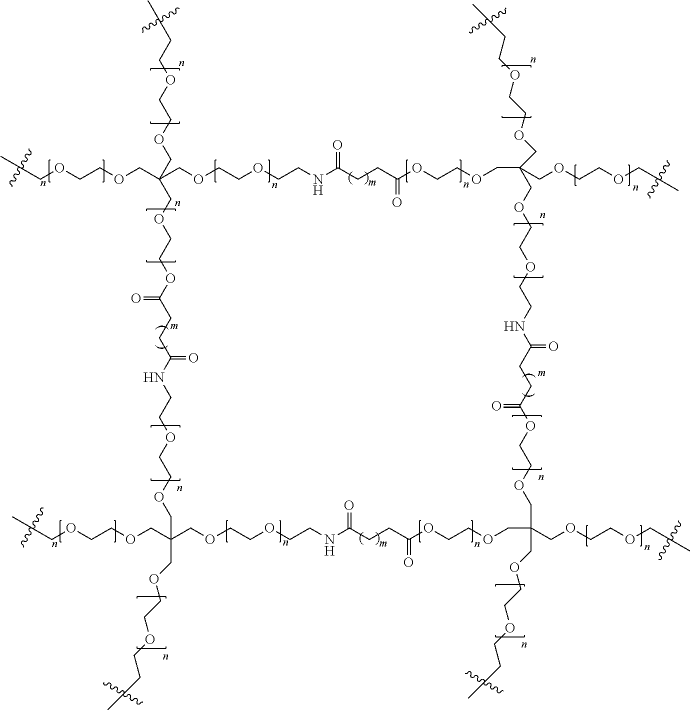

wherein n represents an ethylene oxide repeating unit and the dashed lines represent the points of repeating units of the polymer network.

9. The method of any one of Embodiments 1 to 8, wherein the polymer network is formed by reacting a plurality of polyethylene glycol (PEG) units selected from 4a 20 k PEG-SAZ, 4a 20 kPEG-SAP, 4a 20 kPEG-SG, 4a 20 kPEG-SS, 8a 20 kPEG-SAZ, 8a 20 kPEG-SAP, 8a 20 kPEG-SG, 8a 20 kPEG-SS with one or more PEG or lysine based-amine groups selected from 4a 20 kPEG-NH₂, 8a 20 kPEG-NH₂, and trilysine, or a salt thereof.

10. The method of any one of Embodiments 1 to 9, wherein the polymer network is formed by reacting 4a 20 kPEG-SG with trilysine or a salt thereof.

11. The method of any one of Embodiments 1 to 10, wherein the polymer network is amorphous under aqueous conditions.

12. The method of any one of Embodiments 1 to 11, wherein the polymer network is semi-crystalline in the absence of water.

13. The method of any one of Embodiments 1 to 12, wherein the corticosteroid is dexamethasone.

14. The method of any one of Embodiments 1 to 12, wherein the corticosteroid is dexamethasone formulated for delivery at a dosage of about 0.2 mg to about 0.3 mg.

15. The method of any one of Embodiments 1 to 14, wherein the hydrogel composition is an intracanalicular insert.

16. The method of any one of Embodiments 1 to 15, wherein the hydrogel composition is an intracanalicular insert having a length of about 1.0 mm to about 3.0 mm.

17. The method of any one of Embodiments 1 to 16, wherein the hydrogel composition is fully degraded following release of the corticosteroid.

18. The method of any one of Embodiments 1 to 17, wherein episodic flares of dry eye disease are treated.

19. An intracanalicular insert for treating dry eye, wherein the intracanalicular insert occludes the punctum and delivers a therapeutically effective amount of a steroid for up to 3 weeks.

20. The intracanalicular insert of Embodiments 19, wherein the intracanalicular insert delivers a therapeutically effective amount of a steroid for up to two weeks.

21. The intracanalicular insert of Embodiments 19 or 20, wherein the steroid is dexamethasone.

22. The intracanalicular insert of any one of Embodiments 19 to 21, wherein the steroid is dexamethasone formulated for delivery at a dosage of about 0. 2 mg to about 0.3 mg.

23. The intracanalicular insert of any one of Embodiments 19 to 22, wherein the intracanalicular insert has a length of about 1.0 mm to about 3.0 mm.

24. The intracanalicular insert of any one of Embodiments 19 to 23, wherein the intracanalicular insert is biodegradable.

25. The intracanalicular insert of any one of Embodiments 19 to 24, wherein the intracanalicular insert comprises a hydrogel comprising polymer network having a plurality of polyethylene glycol (PEG) units.

26. The intracanalicular insert of Embodiments 25, wherein the polymer network comprises a plurality of multi-arm PEG units having from 2 to 10 arms.

27. The intracanalicular insert of Embodiments 25, or 26, wherein the polymer network comprises a plurality of multi-arm PEG units having from 4 to 10 arms.

28. The intracanalicular insert of any one of Embodiments 25 to 27, wherein the polymer network comprises a plurality of multi-arm PEG units having from 4 to 8 arms.

29. The intracanalicular insert of any one of Embodiments 25 to 28, wherein the polymer network comprises a plurality of multi-arm PEG units having 8 arms.

30. The intracanalicular insert of any one of Embodiments 25 to 29, wherein the polymer network comprises a plurality of multi-arm PEG units having 4 arms.

31. The intracanalicular insert of any one of Embodiments 25 to 30, wherein the polymer network comprises a plurality of PEG units having the formula:

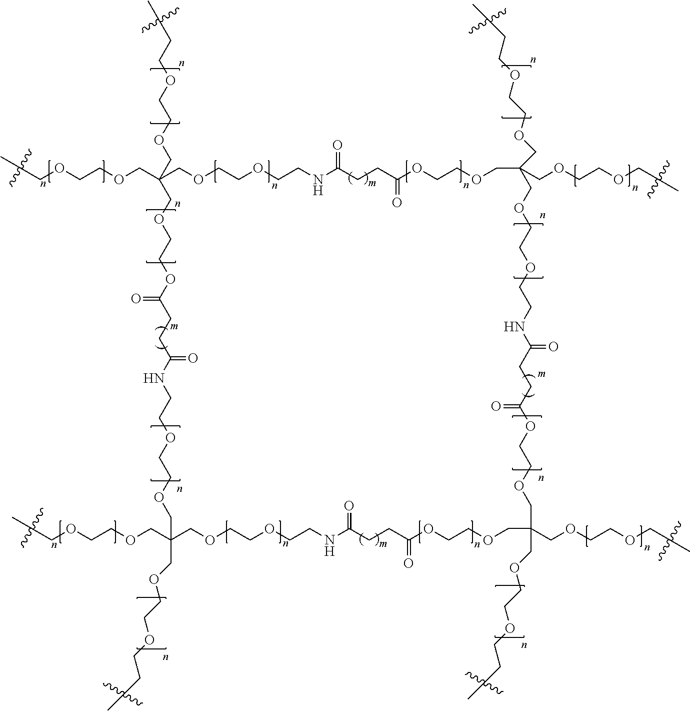

wherein n represents an ethylene oxide repeating unit and the dashed lines represent the points of repeating units of the polymer network.

32. The intracanalicular insert of any one of Embodiments 25 to 31, wherein the polymer network is formed by reacting a plurality of polyethylene glycol (PEG) units selected from 4a 20 k PEG-SAZ, 4a 20 kPEG-SAP, 4a 20 kPEG-SG, 4a 20 kPEG-SS, 8a 20 kPEG-SAZ, 8a 20 kPEG-SAP, 8a 20 kPEG-SG, 8a 20 kPEG-SS with one or more PEG or lysine based-amine groups selected from 4a 20 kPEG-NH₂, 8a 20 kPEG-NH₂, and trilysine, or a salt thereof.

33. The intracanalicular insert of any one of Embodiments 25 to 32, wherein the polymer network is formed by reacting 4a 20 kPEG-SG with trilysine or a salt thereof.

34. The intracanalicular insert of any one of Embodiments 25 to 33, wherein the polymer network is amorphous under aqueous conditions.

35. The intracanalicular insert of any one of Embodiments 25 to 34, wherein the polymer network is semi-crystalline in the absence of water.

36. The intracanalicular insert of any one of Embodiments 25 to 35, wherein the hydrogel is fully degraded following release of the steroid.

### Second List of Embodiments

1. A sustained release biodegradable intracanalicular insert comprising a hydrogel and equal to or less than about 375 μg dexamethasone or an equivalent dose of another glucocorticoid.

2. A sustained release biodegradable intracanalicular insert comprising a hydrogel and a glucocorticoid, wherein the insert in a dry state has an average length of equal to or less than about 2.75 mm.

3. A sustained release biodegradable intracanalicular insert comprising a hydrogel and a glucocorticoid, wherein the insert provides for a release of a therapeutically effective amount of the glucocorticoid for a period of up to about 25 days after administration.

4. The sustained release biodegradable intracanalicular insert of any of the preceding Embodiments, comprising dexamethasone as the glucocorticoid.

5. The sustained release biodegradable intracanalicular insert of any of Embodiments 2, 3 or 4, comprising equal to or less than about 375 μg dexamethasone.

6. The sustained release biodegradable intracanalicular insert of any of the preceding Embodiments, comprising equal to or less than about 350 μg dexamethasone.

7. The sustained release biodegradable intracanalicular insert of Embodiment 6, comprising from about 100 μg to about 350 μg dexamethasone, or from about 150 μg to about 320 μg dexamethasone.

8. The sustained release biodegradable intracanalicular insert of Embodiment 5, comprising from about 160 μg to about 250 μg dexamethasone.

9. The sustained release biodegradable intracanalicular insert of Embodiment 8, comprising from about 180 μg to about 220 μg dexamethasone.

10. The sustained release biodegradable intracanalicular insert of Embodiment 9, comprising about 200 μg dexamethasone.

11. The sustained release biodegradable intracanalicular insert of Embodiment 5, comprising from about 240 μg to about 375 μg dexamethasone.

12. The sustained release biodegradable intracanalicular insert of Embodiment 11, comprising from about 270 μg to about 330 μg dexamethasone.

13. The sustained release biodegradable intracanalicular insert of Embodiment 12, comprising about 300 μg dexamethasone.

14. The sustained release biodegradable intracanalicular insert of any of the preceding Embodiments, wherein the glucocorticoid particles are homogeneously dispersed within the hydrogel.

15. The sustained release biodegradable intracanalicular insert of Embodiment 14, wherein the glucocorticoid particles are micronized particles.

16. The sustained release biodegradable intracanalicular insert of Embodiment 15, wherein the glucocorticoid particles are micronized dexamethasone particles having a d90 particle size of less than about 100 μm, or of less than about 75 μm, or of less than about 50 μm, or of less than about 20 μm, or of less than about 10 μm.

17. The sustained release biodegradable intracanalicular insert of any of the preceding Embodiments, wherein the insert is in a dry state prior to administration and becomes hydrated once administered into the canaliculus.

18. The sustained release biodegradable intracanalicular insert of any of the preceding Embodiments, wherein the hydrogel comprises a polymer network comprising one or more crosslinked polymer units of polyethylene glycol, polyethylene oxide, polypropylene oxide, polyvinyl alcohol, poly (vinylpyrrolidinone), polylactic acid, polylactic-co-glycolic acid, random or block copolymers or combinations or mixtures of any of these, or one or more units of polyaminoacids, glycosaminoglycans, polysaccharides, or proteins.

19. The sustained release biodegradable intracanalicular insert of Embodiment 18, wherein the polymer network comprises one or more crosslinked polyethylene glycol units having an average molecular weight in the range from about 2,000 to about 100,000 Daltons, or from about 10,000 to about 60,000 Daltons, or from about 15,000 to about 50,000 Daltons.

20. The sustained release biodegradable intracanalicular insert of Embodiment 19, wherein the polyethylene glycol units have an average molecular weight of about 20,000 Daltons.

21. The sustained release biodegradable intracanalicular insert of any of Embodiments 18 to 20, wherein the polymer network comprises one or more crosslinked 2- to 10-arm polyethylene glycol units, or one or more 4- to 8-arm polyethylene glycol units.

22. The sustained release biodegradable intracanalicular insert of Embodiment 21, wherein the polymer network comprises 4-arm polyethylene glycol units.

23. The sustained release biodegradable intracanalicular insert of any of Embodiments 18 to 22, wherein the polymer network is formed by reacting an electrophilic group-containing multi-arm-polymer precursor with a nucleophilic group-containing crosslinking agent.

24. The sustained release biodegradable intracanalicular insert of Embodiment 23, wherein the nucleophilic group is an amine group and the electrophilic group is an activated ester group.

25. The sustained release biodegradable intracanalicular insert of Embodiment 24, wherein the electrophilic group-containing multi-arm-polymer precursor is 4a 20 kPEG-SG and the crosslinking agent is trilysine acetate.

26. The sustained release biodegradable intracanalicular insert of any of the preceding Embodiments, containing a visualization agent.

27. The sustained release biodegradable intracanalicular insert of Embodiment 26, wherein the visualization agent is a fluorophore.

28. The sustained release biodegradable intracanalicular insert of Embodiment 27, wherein the visualization agent is fluorescein.

29. The sustained release biodegradable intracanalicular insert of any of the preceding Embodiments, wherein the insert in a dry state contains from about 40% to about 56% by weight of the glucocorticoid and from about 36% to about 55% by weight polymer units.

30. The sustained release biodegradable intracanalicular insert of Embodiment 29, wherein the insert in a dry state contains from about 40% to about 46% by weight dexamethasone and from about 45% to about 55% by weight polyethylene glycol units.

31. The sustained release biodegradable intracanalicular insert of Embodiment 29, wherein the insert in a dry state contains from about 50% to about 56% by weight dexamethasone and from about 36% to about 46% by weight polyethylene glycol units.

32. The sustained release biodegradable intracanalicular insert of any of Embodiments 26 to 31, wherein the insert in a dry state contains from about 0.1% to about 1% by weight visualization agent.

33. The sustained release biodegradable intracanalicular insert of any of the preceding Embodiments, wherein the insert in a dry state contains one or more phosphate, borate or carbonate salt(s).

34. The sustained release biodegradable intracanalicular insert of Embodiment 33, wherein the insert contains from about 0.5% to about 5% by weight of one or more phosphate salt(s).

35. The sustained release biodegradable intracanalicular insert of any of the preceding Embodiments, wherein the insert in a dry state contains no more than about 1% by weight water.

36. The sustained release biodegradable intracanalicular insert of any of the preceding Embodiments, wherein the insert is free or substantially free of anti-microbial preservatives.

37. The sustained release biodegradable intracanalicular insert of any of the preceding Embodiments, wherein the insert has an essentially cylindrical shape.

38. The sustained release biodegradable intracanalicular insert of any of Embodiments 1 or 3 to 37, wherein the insert in a dry state has an average length of less than about 3 mm.

39. The sustained release biodegradable intracanalicular insert of Embodiment 38, wherein the insert in a dry state has an average length of equal to or less than about 2.75 mm.

40. The sustained release biodegradable intracanalicular insert of Embodiment 2 or 39, wherein the insert in a dry state has an average length of equal to or less than about 2.5 mm.

41. The sustained release biodegradable intracanalicular insert of any of the preceding Embodiments, wherein the insert in a dry state has an average diameter of less than about 1 mm, or an average diameter of less than about 0.75 mm.

42. The sustained release biodegradable intracanalicular insert of any of the preceding Embodiments, wherein the insert in a dry state has an average length in the range of about 2.14 mm to about 2.36 mm and an average diameter in the range of about 0.41 mm to about 0.55 mm.

43. The sustained release biodegradable intracanalicular insert of Embodiment 42, wherein the insert in a dry state has an average length of about 2.25 mm and an average diameter of about 0.5 mm.

44. The sustained release biodegradable intracanalicular insert of any of the preceding Embodiments, wherein upon hydration in vivo in the canaliculus or in vitro the average diameter of the insert is increased and optionally its average length is decreased.

45. The sustained release biodegradable intracanalicular insert of Embodiment 44, wherein hydration in vitro is measured in phosphate-buffered saline at a pH of 7.4 at 37° C. after 24 hours.

46. The sustained release biodegradable intracanalicular insert of Embodiment 44 or 45, wherein upon hydration the average diameter of the insert is increased by a factor in the range of about 1.5 to about 4, or in the range of about 2 to about 3.5.

47. The sustained release biodegradable intracanalicular insert of Embodiment 46, wherein upon hydration the average diameter of the insert is increased by a factor of about 3.

48. The sustained release biodegradable intracanalicular insert of any of Embodiments 44 to 47, wherein upon hydration the average length of the insert is decreased to about 0.9 times its average length in the dry state or less, or to about 0.75 times its average length in the dry state or less.

49. The sustained release biodegradable intracanalicular insert of Embodiment 48, wherein upon hydration the average length of the insert is decreased to about two-thirds of its average length in the dry state.

50. The sustained release biodegradable intracanalicular insert of any of the preceding Embodiments, wherein the insert in a hydrated state has an average diameter in the range of about 1 mm to about 2 mm and an average length that is shorter than the average length of the insert in the dry state.

51. The sustained release biodegradable intracanalicular insert of Embodiment 50, wherein the insert in a hydrated state has an average diameter in the range of about 1.35 mm to about 1.80 mm and a ratio of length to diameter of greater than 1.

52. The sustained release biodegradable intracanalicular insert of Embodiment 51, wherein the insert in a hydrated state has an average diameter in the range of about 1.40 mm to about 1.60 mm and an average length in the range of about 1.70 mm to about 2.0 mm.

53. The sustained release biodegradable intracanalicular insert of any of the preceding Embodiments, wherein the insert provides for a release of a therapeutically effective amount of glucocorticoid for a period of about 6 hours or longer after administration.

54. The sustained release biodegradable intracanalicular insert of Embodiment 53, wherein the insert provides for a release of a therapeutically effective amount of glucocorticoid for a period of about 12 hours or longer after administration.

55. The sustained release biodegradable intracanalicular insert of any of Embodiments 1, 2 or 4 to 54, wherein the insert provides for a release of a therapeutically effective amount of glucocorticoid of up to about 14 days, or up to about 21 days, or up to about 25 days, or up to about 1 month after administration.

56. The sustained release biodegradable intracanalicular insert of Embodiment 3 or 55, wherein the insert provides for a release of a therapeutically effective amount of glucocorticoid for a period of up to about 14 days, or up to about 21 days, or up to about 25 days after administration.

57. The sustained release biodegradable intracanalicular insert of any of the preceding Embodiments, wherein the insert contains about 200 μg dexamethasone and provides for a release of dexamethasone for a period of up to about 14 days after administration.

58. The sustained release biodegradable intracanalicular insert of any of the preceding Embodiments, wherein the insert contains about 300 μg dexamethasone and provides for a release of dexamethasone for a period of up to about 21 days after administration.

59. The sustained release biodegradable intracanalicular insert of any of the preceding Embodiments, wherein the insert provides for an average release from about 15 μg to about 25 μg dexamethasone per day during a period of up to about 7 days, or up to about 14 days, or up to about 21 days after administration.

60. The sustained release biodegradable intracanalicular insert of any of Embodiments 1 to 57, wherein the insert contains about 200 μg dexamethasone and provides for an average release from about 15 μg to about 25 μg dexamethasone per day during a period of up to about 7 days after administration.

61. The sustained release biodegradable intracanalicular insert of any of Embodiments 1 to 56 or 58, wherein the insert contains about 300 μg dexamethasone and provides for an average release from about 15 μg to about 25 μg dexamethasone per day during a period of up to about 11 days, or up to about 14 days after administration.

62. The sustained release biodegradable intracanalicular insert of any of the preceding Embodiments, wherein after complete depletion of glucocorticoid from the insert the hydrogel is biodegraded in the canaliculus and/or cleared through the nasolacrimal duct within about 1 month, or within about 2 months, or within about 3 months, or within about 4 months, after administration.

63. The sustained release biodegradable intracanalicular insert of any of Embodiments 1 to 61, wherein the hydrogel is biodegraded prior to complete depletion of glucocorticoid from the insert.

64. A sustained release biodegradable intracanalicular insert comprising a hydrogel and dexamethasone particles dispersed within the hydrogel, wherein the insert contains from about 160 μg to about 250 μg or from about 180 μg to about 220 μg or about 200 μg dexamethasone and has in the dry state an average diameter in the range of about 0.41 mm to about 0.49 mm and an average length in the range of about 2.14 mm to about 2.36 mm, and has in the hydrated state an average diameter in the range of about 1.35 mm to about 1.80 mm and a ratio of length to diameter of greater than 1, and wherein the insert provides for a release of dexamethasone for a period of up to about 14 days, or up to about 21 days, after administration.

65. A sustained release biodegradable intracanalicular insert comprising a hydrogel and dexamethasone particles dispersed within the hydrogel, wherein the insert contains from about 240 μg to about 375 μg or from about 270 μg to about 330 μg or about 300 μg dexamethasone and has in the dry state an average diameter in the range of about 0.44 mm to about 0.55 mm and an average length in the range of about 2.14 mm to about 2.36 mm, and has in the hydrated state an average diameter in the range of about 1.35 mm to about 1.80 mm and a ratio of length to diameter of greater than 1, and wherein the insert provides for a release of dexamethasone for a period of up to about 21 days, or up to about 1 month, after administration.

66. The sustained release biodegradable intracanalicular insert of Embodiment 64 or 65, wherein the hydrogel comprises a polymer network comprising units of a crosslinked multi-arm polyethylene glycol and a visualization agent.

67. The sustained release biodegradable intracanalicular insert of any of Embodiments 64 to 66, wherein the polymer network comprises 4a 20 kPEG-SG units that have been crosslinked with trilysine acetate.

68. The sustained release biodegradable intracanalicular insert of any of Embodiments 64 to 67, wherein the visualization agent is fluorescein.

69. A method of manufacturing a sustained release biodegradable intracanalicular insert according to any of the preceding Embodiments, the method comprising the steps of forming a hydrogel comprising a polymer network and glucocorticoid particles dispersed in the hydrogel, shaping the hydrogel and drying the hydrogel.

70. The method of Embodiment 69, wherein the glucocorticoid is dexamethasone.

71. The method of Embodiment 69 or 70, wherein the glucocorticoid particles are homogeneously dispersed within the hydrogel.

72. The method of any of Embodiments 69 to 71, wherein the glucocorticoid particles are micronized particles.

73. The method of Embodiment 71 or 72, wherein the glucocorticoid particles are micronized dexamethasone particles having a d90 particle size of less than about 100 μm, or of less than about 75 μm, or of less than about 50 μm, or of less than about 20 μm, or of less than about 10 μm and are homogeneously dispersed within the hydrogel.

74. The method of any of Embodiments 69 to 73, wherein equal to or less than about 375 μg, or equal to or less than about 350 μg, or from about 100 μg to about 350 μg, or from about 150 μg to about 320 μg dexamethasone are contained in the insert.

75. The method of Embodiment 74, wherein from about 160 μg to about 250 μg dexamethasone, or from about 180 μg to about 220 μg dexamethasone, or about 200 μg dexamethasone are contained in the insert.

76. The method of Embodiment 74, wherein from about 240 μg to about 375 μg dexamethasone, or from about 270 μg to about 330 μg dexamethasone, or about 300 μg dexamethasone are contained in the insert.

77. The method of any of Embodiments 69 to 76, wherein the polymer network is formed from one or more crosslinked polymer units of polyethylene glycol, polyethylene oxide, polypropylene oxide, polyvinyl alcohol, poly (vinylpyrrolidinone), polylactic acid, polylactic-co-glycolic acid, random or block copolymers or combinations or mixtures of any of these, or one or more units of polyaminoacids, glycosaminoglycans, polysaccharides, or proteins.

78. The method of any of Embodiments 69 to 77, wherein the polymer network is formed by crosslinking multi-arm polyethylene glycol units in a buffered solution.

79. The method of Embodiment 78, wherein the polymer network is formed by mixing and reacting an electrophilic group-containing multi-arm polyethylene glycol with a nucleophilic group-containing crosslinking agent in a buffered solution in the presence of dexamethasone, and allowing the mixture to gel.

80. The method of Embodiment 79, wherein the crosslinking agent contains amine groups.

81. The method of Embodiment 79 or 80, wherein the electrophilic group-containing multi-arm-polymer precursor is 4a 20 kPEG-SG and the crosslinking agent is trilysine acetate.

82. The method of any of Embodiments 69 to 81, comprising admixing a visualization agent.

83. The method of Embodiments 82, comprising conjugating the visualization agent with the polymer network.

84. The method of Embodiment 83, comprising conjugating the visualization agent with the crosslinking agent prior to crosslinking the polymer precursor.

85. The method of any of Embodiments 82 to 84, wherein the visualization agent is a fluorophore.

86. The method of Embodiments 85, wherein the visualization agent is fluorescein.

87. The method of Embodiment 86, wherein the fluorescein is conjugated to the trilysine acetate prior to the crosslinking reaction.

88. The method of any of Embodiments 81 to 87, wherein the molar ratio of 4a 20 kPEG-SG to trilysine acetate is from about 1:2 to about 2:1.

89. The method of any of Embodiments 79 to 88, wherein the method comprises the steps of filling the mixture into a mold or tubing prior to complete gelling of the hydrogel, allowing the mixture to gel, and drying the hydrogel.

90. The method of Embodiment 89, wherein the mixture is filled into a fine diameter tubing in order to prepare a hydrogel strand.

91. The method of Embodiment 90, wherein the inside of the tubing has a round geometry.

92. The method of Embodiment 90 or 91, wherein the method further comprises stretching the hydrogel strand.

93. The method of Embodiment 92, wherein the stretching of the hydrogel strand is performed prior to drying the hydrogel strand.

94. The method of Embodiment 93, wherein the hydrogel strand is stretched by a stretch factor in the range of about 1.5 to about 3, or of about 2.2 to about 2.8, or of about 2.5 to about 2.6.

95. The method of any of Embodiments 90 to 94, wherein the dry hydrogel strand is cut into segments having an average length of equal to or less than about 2.75 mm.

96. The method of Embodiment 95, wherein the dry hydrogel strand is cut into segments having an average length of equal to or less than about 2.5 mm.

97. The method of Embodiment 96, wherein the hydrogel strand is cut into segments of about 2.25 mm average length.

98. A method of treating dry eye disease in a patient in need thereof, the method comprising administering to the patient a sustained release biodegradable intracanalicular insert according to any of Embodiments 1 to 68 or manufactured in accordance with the method of any of Embodiments 69 to 97.

99. The method of Embodiment 98, wherein the treatment is an acute treatment of dry eye disease.

100. The method of Embodiment 98 or 99, wherein the treatment is an acute treatment of episodic flares of dry eye disease.

101. The method of any of Embodiments 98 to 100, wherein the insert is administered to the inferior and/or superior canaliculus.

102. The method of any of Embodiments 98 to 100, wherein the insert is administered to the vertical canaliculus.

103. The method of any of Embodiments 98 to 102, wherein the insert is administered bilaterally.

104. The method of any of Embodiments 98 to 103, wherein the glucocorticoid is delivered to the ocular surface through the tear film.

105. The method of any of Embodiments 98 to 104, wherein the insert contains a dose of equal to or less than about 375 μg dexamethasone or an equivalent dose of another glucocorticoid.

106. The method of Embodiment 105, wherein the insert contains a dose of equal to or less than about 350 μg dexamethasone or an equivalent dose of another glucocorticoid.

107. The method of any of Embodiments 98 to 106, wherein the insert contains dexamethasone.

108. The method of Embodiment 107, wherein the insert contains from about 100 μg to about 350 μg dexamethasone.

109. The method of Embodiment 105, wherein the insert contains from about 160 μg to about 250 μg dexamethasone, or from about 180 μg to about 220 μg dexamethasone, or about 200 μg dexamethasone.

110. The method of Embodiment 105, wherein the insert contains from about 240 μg to about 375 μg dexamethasone, or from about 270 μg to about 330 μg dexamethasone, or about 300 μg dexamethasone.

111. The method of any of Embodiments 98 to 110, wherein the insert releases a therapeutically effective amount of dexamethasone for a period of about 6 hours or longer after administration.

112. The method of Embodiment 111, wherein the insert releases a therapeutically effective amount of dexamethasone for a period of about 12 hours or longer after administration.

113. The method of any of Embodiments 98 to 112, wherein the insert releases a therapeutically effective amount of dexamethasone for a period of up to about 7 days, or up to about 14 days, or up to about 21 days, or up to about 25 days, or up to about 1 month after administration.

114. The method of Embodiment 113, wherein the insert contains about 200 μg dexamethasone and releases dexamethasone for a period of up to about 14 days after administration.

115. The method of Embodiment 113, wherein the insert contains about 300 μg dexamethasone and releases dexamethasone for a period of up to about 21 days after administration.

116. The method of any of Embodiments 98 to 115, wherein the insert releases on average about 15 μg to about 25 μg dexamethasone per day for a period of up to about 14 days, or up to about 21 days after administration.

117. The method of Embodiment 116, wherein the insert contains about 200 μg dexamethasone and releases on average about 15 μg to about 25 μg dexamethasone per day for a period of up to about 7 days after administration.

118. The method of Embodiment 116, wherein the insert contains about 300 μg dexamethasone and releases on average about 15 μg to about 25 μg dexamethasone per day for a period of up to about 11 days, or up to about 14 days after administration.

119. The method of any of Embodiments 98 to 118, wherein after complete depletion of the glucocorticoid from the insert the insert remains in the canaliculus until the hydrogel has biodegraded and/or is cleared through the nasolacrimal duct.

120. The method of Embodiment 119, wherein the insert remains in the canaliculus for up to about 1 month, or up to about 2 months, or up to about 3 months, or up to about 4 months, after administration.

121. The method of any of Embodiments 98 to 120, comprising the administration of a further sustained release biodegradable intracanalicular insert according to any of claims 1 to 68 or manufactured in accordance with the method of any of claims 69 to 97 into the canaliculus while the first insert is still retained in the canaliculus (“insert stacking”).

122. The method of Embodiment 121, wherein the further insert is the same or is different from the first insert.

123. The method of Embodiment 121 or 122, wherein the further insert is administered when the first insert has been completely depleted of glucocorticoid.

124. The method of any of Embodiments 121 to 122, wherein the further insert is inserted when the first insert has not yet been completely depleted of glucocorticoid.

125. The method of any of Embodiments 98 to 124, wherein the treatment of dry eye disease by means of the sustained release biodegradable intracanalicular insert is combined with or followed up by another treatment of dry eye disease.

126. The method of Embodiment 125, wherein the other treatment of dry eye disease is a chronic treatment of dry eye disease.

127. The sustained release biodegradable intracanalicular insert according to any of Embodiments 1 to 68 or manufactured in accordance with the method of any of Embodiments 69 to 97 for use in treating dry eye disease in a patient in need thereof according to the method of any of Embodiments 98 to 126.

128. Use of a sustained release biodegradable intracanalicular insert according to any of Embodiments 1 to 68 or manufactured in accordance with the method of any of Embodiments 69 to 97 for the manufacture of a medicament for treating dry eye disease in a patient in need thereof according to the method of any of Embodiments 98 to 126.

129. A kit comprising one or more sustained release biodegradable intracanalicular insert(s) according to any of Embodiments 1 to 68 or manufactured in accordance with the method of any of Embodiments 69 to 97 and instructions for using the insert(s).

130. The kit according to Embodiment 129, further comprising one or more means for administration of the insert(s).

131. The kit according to Embodiment 129 or 130, wherein the insert(s) is/are individually packaged for a single administration.

132. The kit according to any one of Embodiments 129 to 131, wherein the insert(s) is/are fixated in a foam carrier which is sealed in a foil pouch.

### EXAMPLES

The following Examples are included to demonstrate certain aspects and embodiments of the invention as described in the claims. It should be appreciated by those of skill in the art, however, that the following description is illustrative only and should not be taken in any way as a restriction of the invention.

### Example 1: Preparation of Dexamethasone Inserts

The dexamethasone inserts of some embodiments of the present application are essentially cylindrical having a certain length and diameter as specified herein, with dexamethasone homogeneously dispersed and entrapped within a PEG-based hydrogel matrix to provide sustained release of dexamethasone to the ocular surface through the tear fluid. The release of dexamethasone from the inserts of the invention is solubility-driven, as dexamethasone has a low aqueous solubility.

For preparation of the inserts used in the examples, an autoclaved polyurethane tubing was cut into appropriate length pieces first. The formulation process involved preparing one syringe containing trilysine acetate and NHS fluorescein and another syringe containing dexamethasone and 4a 20 k PEG-SG (4-arm 20,000 Da PEG succinimidyl glutarate ester). The contents of these two syringes are then combined to form a mixture (a suspension), which is allowed to gel to form the hydrogel with dexamethasone dispersed therein. In the following, the production process is described exemplarily for inserts comprising 0.2 mg or 0.3 mg dexamethasone.

For preparation of the trilysine acetate/NHS fluorescein syringe, corresponding amounts of buffer (sodium phosphate dibasic), water for injection, and trilysine acetate were mixed and the pH was adjusted to 8.4 (Table 2). Afterwards, 16,045±10 mg of the resulting solution were mixed with corresponding amounts of NHS-fluorescein (Table 2). The trilysine acetate and NHS-fluorescein mixture was allowed to react for 1 to 24 hours at room temperature to form a trilysine-fluorescein conjugate. After the reaction time had elapsed (and formation of the trilysine-fluorescein conjugate was confirmed using RP-HPLC with UV detection), the solution was filtered and a 4,200±10 mg portion of the prepared solution was aliquoted into a syringe.

TABLE 2
Ingredients of the trilysine acetate/NHS fluorescein syringe.

| | Amount per Preparation | |
|---|---|---|
| Ingredient | For the 0.3 mg insert | For the 0.2 mg insert |
| Water for Injection | 26.0 ± 0.1 g | 26.0 ± 0.1 g |
| Sodium Phosphate Dibasic | 650 ± 5 mg | 650 ± 5 mg |
| Trilysine Acetate | 260 ± 5 mg | 260 ± 5 mg |
| NHS-Fluorescein | 50.0 ± 1.0 mg | 50.0 ± 1.0 mg |

For preparation of the dexamethasone/4a 20 k PEG-SG syringe two separate syringes were prepared first and then combined to produce the dexamethasone/4a 20 k PEG-SG syringe. The first syringe contained a suspension of corresponding amounts of sieved, micronized dexamethasone (Pfizer) in water (Table 3). The dexamethasone had a particle size of d90≤5 μm and d98<10 μm, and had been additionally sieved to remove particles that are 90 μm or larger. The second syringe contained 5,620±10 mg of a solution of 4a 20 k PEG-SG and sodium phosphate monobasic buffer solution that was prepared by mixing the corresponding amounts (Table 3) in a sterile container. The dexamethasone suspension syringe was then connected to the 4a 20 k PEG-SG syringe luer-to-luer and the contents of the syringes were mixed by passing back and forth between each syringe. The suspension was then transferred into one single syringe to form the dexamethasone/4a 20 k PEG-SG syringe.

TABLE 3
Ingredients of the dexamethasone/4a20k PEG-SG syringe.

| | Amount per Preparation | |
|---|---|---|
| Ingredient | For the 0.3 mg insert | For the 0.2 mg insert |
| Water for Injection | 7.4 ± 0.1 g | 7.4 ± 0.1 g |
| Micronized Dexamethasone | 1,879 ± 5 mg | 1,203 ± 5 mg |
| (sieved) | | |
| Sodium Phosphate | 6,350 ± 100 mg | 6,350 ± 100 mg |
| Monobasic Solution | | |
| 4a20kPEG-SG | 2,200 ± 5 mg | 2,200 ± 5 mg |

The trilysine acetate/NHS fluorescein syringe and the dexamethasone/4a 20 k PEG-SG syringe were connected luer-to-luer and mixed by passing the contents of the syringes back and forth between each syringe, creating a mixture (suspension) of the hydrogel components and dexamethasone, which mixture was then transferred into a single syringe. The suspension was cast through the prepared polyurethane tubing before (complete) gelling of the hydrogel. Gelling time was confirmed by performing a gel tap test. The casted strands were stored vertically for 3 to 6 hours to allow the hydrogel to cure. Afterwards, the strands were stretched at a controlled rate to approximately 2.5-2.6 times the original tubing length. The stretched strands were stored vertically in nitrogen-flashed atmosphere for 60 to 72 hours at 32.0±2.0° C. to allow the strands to dry completely.

After drying, the dried strands were removed from the polyurethane tubing and cut into approximately 2.25 mm segments. The surface of the cut inserts was inspected for particulate, cylindrical shape and any visible surface defects. Inserts that did not show any defects and provided the required shape were evaluated for dimensional length and diameter. Inserts that did not meet all requirements were rejected.

After quality inspection, the inserts were packaged separately into foam carrier (one insert per foam carrier) and sealed in an aluminum-low density polyethylene (LDPE) foil pouch that can be peeled open by the user (FIG. 1). For this, an insert was placed with forceps into the opening in a foam carrier with a portion of the insert protruding for easy removal. The foam carrier with insert was placed into the foil pouch. The unsealed foil pouches were transferred into a glovebox and kept there for 16-96 hours in an inert nitrogen environment to reduce residual moisture from the foam and pouch material (moisture content≤1.0%). The pouches were then sealed within the glovebox using a pouch sealer to create a complete, continuous seal on the pouch. After the pouches were sealed, they were inspected and stored at 2-8° C. until sterilization. For sterilization, packaged inserts were gamma irradiated (internal dose delivered 25.0-45.1 kGy). Afterwards, the packaged inserts were stored protected from light at 2-8° C. prior to administration.

The percent composition, target quantities per insert and the function of each ingredient are presented in Table 4 for both the 0.2 mg and the 0.3 mg dexamethasone inserts.

TABLE 4
Dexamethasone insert composition (0.2 mg and 0.3 mg dose).
Percentages refer to percent by weight (% w/w).

| | 0.2 mg Insert | | 0.3 mg Insert | | |
|---|---|---|---|---|---|
| | Composition | Target Quantity | Composition | Target | |
| Ingredient | (%) | (μg) | (%) | Quantity (μg) | Function |
| Micronized | 43.73 | 200 | 53.83 | 300 | Active |
| Dexamethasone | | | | | Pharmaceutical |
| | | | | | Ingredient |
| 4a20kPEG-SG | 49.64 | 227 | 40.73 | 227 | Hydrogel matrix |
| Trilysine Acetate | 1.39 | 6.36 | 1.14 | 6.36 | Hydrogel matrix |
| NHS-Fluorescein | 0.45 | 2.06 | 0.37 | 2.06 | Visualization |
| | | | | | agent |
| Sodium Phosphate | 3.48 | 15.9 | 2.85 | 15.9 | Buffer salt |
| Dibasic | | | | | |
| Sodium Phosphate | 1.31 | 5.99 | 1.07 | 5.99 | Buffer salt |
| Monobasic | | | | | |
| Anhydrous | | | | | |
| Total Insert | 100 | 457.31 | 100 | 557.31 | |

Characteristics of the 0.2 mg and the 0.3 mg dexamethasone inserts are presented in Table 5. The duration of dexamethasone release is estimated to last for up to about 14 days for the 0.2 mg insert and up to about 21 days for the 0.3 mg insert (see also Example 3 below). The hydrated dimensions were measured as disclosed herein after 24 hours in biorelevant media (phosphate buffered saline (PBS), pH 7.4 at 37° C.), which is considered equilibrium. Measurement of insert dimensions (both in the dry and in the wet state) were performed by a custom 3-camera Keyence Inspection System. 2 Cameras were used to measure the diameter with a tolerance of ±0.002 mm (of all datapoints acquired, the average (=mean) value is recorded), and 1 camera was used to measure the length with a tolerance of ±0.04 mm (of several datapoints, the longest measured length is recorded).

TABLE 5
Dexamethasone insert characteristics (0.2 mg and 0.3 mg dose).
Average dimensional values indicate the mean from measurements of 22 inserts.

| Characteristics | 0.2 mg Insert | 0.3 mg Insert |
|---|---|---|
| Dose | 0.2 mg dexamethasone per insert | 0.3 mg dexamethasone per insert |
| Appearance | Light yellow to yellow/orange | Light yellow to yellow/orange |
| Dry Dimensions | Diameter: 0.41-0.49 mm | Diameter: 0.44-0.55 mm |
| | (Average: 0.45 mm) | (Average: 0.50 mm) |
| | Length: 2.14-2.36 mm | Length: 2.14-2.36 mm |
| | (Average: 2.25 mm) | (Average: 2.25 mm) |
| Hydrated (“Wet”) | Diameter: 1.35-1.80 mm | Diameter: 1.35-1.80 mm |
| Dimensions | (Average: 1.54 mm) | (Average: 1.47 mm) |
| | Length: 1.69-1.87 mm | Length: 1.64-2.00 mm |
| | (Average: 1.79 mm) | (Average: 1.85 mm) |
| Intended Duration of | About 14 days | About 21 days |
| Release | | |

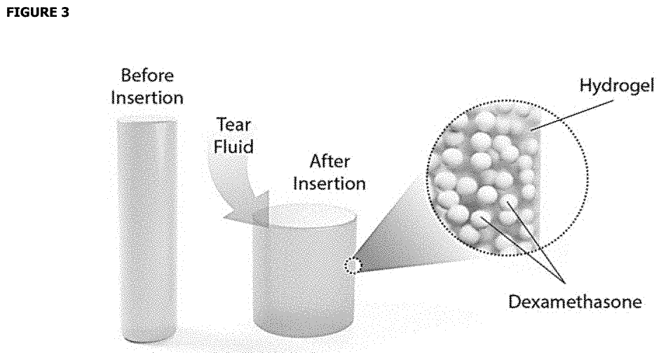

Inserts are intended for administration through the upper and/or lower punctum of the eye into the superior and/or inferior vertical canaliculus of the eye using e.g. tweezers (FIG. 2A). The insert can be visualized by illuminating the fluorescent PEG with a blue light source and using a yellow filter (FIG. 2B). Stretching of the strands during the production process creates a shape memory, meaning that the insert upon hydration when administered into the canaliculus of the eye will rapidly shrink in length (e.g. to about ⅔ of the length in the dry state) and widen in diameter (e.g. to about 3× the diameter in the dry state) to approach its original wet casted dimension (FIG. 3 and Table 5). In general, the degree of shrinking in length and expanding in diameter upon hydration depends inter alia on the stretch factor. While the narrow dry dimensions facilitate administration of the insert through the punctum into the canaliculus, the shortened length after administration yields a shorter insert in the canaliculus of the eye minimizing potentially disturbing effects for the patient and provides for a good fit and thus a good retention in the vertical canaliculus. In addition, as the insert's diameter expands upon hydration, it thereby adapts to and closely fits within the individual canaliculus size of the patient. Thereby, unintended loss of the insert as sometimes experienced with commonly used plugs such as collagen or silicon plugs is greatly reduced.

After placement of the insert in the canaliculus, the micronized dexamethasone contained in the insert dissolves in tear fluid to provide a sustained topical delivery of therapeutically effective amounts of dexamethasone to the ocular surface. The release of a therapeutically effective amount of dexamethasone lasts, for instance, up to about 14 days in case of the 0.2 mg insert and up to about 21 days in case of the 0.3 mg insert. After all dexamethasone has been released from the insert, the dexamethasone-depleted insert remains in the canaliculus for a certain period of time, such as for about 1, about 2, about 3, or about 4 months after administration, and is slowly biodegraded and becomes smaller until it is cleared (disposed/washed out) through the nasolacrimal duct. As only one administration is required for prolonged delivery times of up to several weeks, patient compliance is increased as compared to the use of eye drops that have to be administered daily or even several times per day. Dexamethasone is released primarily from the proximal end of the insert at the interface between the hydrogel and the tear fluid (as shown exemplarily in FIG. 6). The sustained drug release rate is controlled by drug solubility in the hydrogel matrix and the tear fluid. The hydrogel matrix of the insert is formulated to biodegrade e.g. via ester hydrolysis in the aqueous environment of the canaliculus. Thereby, over time, the insert softens, liquefies and is cleared (disposed/washed out) through the nasolacrimal duct without the need for removal (unless removal is desired in a particular circumstance). Unpleasant removal can thus be avoided. The inserts are applied for the treatment of signs and symptoms of dry eye disease (DED), in particular for the acute treatment of DED, for instance, upon episodic flares of DED. Therefore, the dexamethasone inserts of the present application combine the effect of inflammation suppression due to dexamethasone release with benefits from lacrimal occlusion, wherein the combined effects provide an improved treatment of DED.

### Example 2: In Vitro Dexamethasone Release

The release rate of dexamethasone from the 0.2 mg and 0.3 mg inserts (Table 4 and 5) was determined by in vitro testing. In vitro release was examined under accelerated conditions as briefly described in the following: One insert was placed into a bottle and 100 mL of buffer solution were added (1× phosphate buffer saline, PBS at pH 7.4) in order to expose the entire insert surface to buffer solution. At corresponding time-points, 1 mL of supernatant was removed for HPLC analysis. 1 mL of fresh buffer solution was added as replacement to the bottle. The in vitro assays can be used e.g. for quality control to determine batch-to-batch conformity of the inserts.

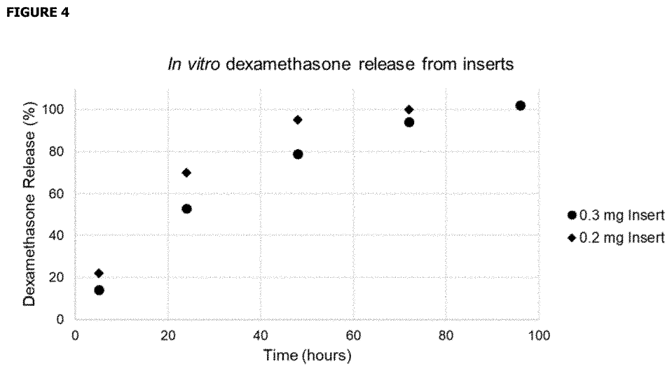

Dexamethasone was completely released after 3 days from the 0.2 mg insert and after 4 days from the 0.3 mg insert (FIG. 4).

### Example 3: Evaluation of Dexamethasone Inserts in Pre-Clinical Studies

Safety, tolerability, and drug release of the dexamethasone inserts comprising varying doses of the active ingredient were evaluated in beagle dogs.

### Determination of Dexamethasone by LC-MS/MS

Dexamethasone concentration in plasma, aqueous humor and tear fluid samples were determined by high performance liquid chromatography combined with tandem mass spectrometry (LC-MS/MS) using a triple quadrupole mass spectrometer.

For preparation of tear fluid samples, deionized water was added to the tear fluid samples to obtain a volume of 50 μL for each tear sample. Then, 50 μL of internal standard solution (prednisolone-21 acetate) were added to each tear sample. For preparation of aqueous humor samples, 50 μL of each aqueous humor sample were mixed with 50 μL internal standard solution. Samples were centrifuged at 13,500 rpm for 5 min. For preparation of plasma samples, 50 μL beagle plasma were mixed with 200 μL internal standard solution in acetonitrile with 0.1% formic acid (v/v). Plasma samples were vortexed and then centrifuged at 4,000 rpm for 15 min. The different sample supernatants were used for LC-MS/MS analysis.

The high performance liquid chromatography (HPLC) system consisted of Shimadzu AD10vp pumps and a CTC autosampler. The mass spectrometer (MS) was an ABI 3000 tandem mass spectrometer. The instruments were operated by Analyst 1.4.2 software. The HPLC mobile phases were acetonitrile and HPLC-grade water with 0.1% formic acid (v/v). The column was kept at ambient temperature, the sample compartment was kept at 2-5° C. The analytes were eluted from the column at 0.8 mL/min using a gradient resulting from mixture of the mobile phases. Dexamethasone was ionized by negative ion electrospray. The MS system was operated at negative ion mode. Dexamethasone (391.0-361.1 m/z; retention time 1.23±0.5 min) and the internal standard (prednisolone-21 acetate, 401.2-321.0 m/z; 1.29±0.5 min) were fragmented in the MS. The total run time was 2.4 min. Dexamethasone concentration was determined from a calibration curve. Prior to analysis of the samples, the method was validated using dexamethasone-comprising beagle plasma and artificial tears. The method was shown to be reproducible, precise, linear, accurate and specific. The lower limit of quantification was determined to be 1.0 ng/mL, the lower limit of detection to be 0.08-0.06 ng/mL.

### Drug Release from Inserts

In order to examine dexamethasone release from inserts according to the present invention comprising different dexamethasone doses, inserts comprising 0.22, 0.37, 0.46, 0.58, 0.65, 0.72, and 0.85 mg dexamethasone, respectively, were administered intracanalicularly to healthy beagle dogs (n=10-14 per dose). Tear fluid samples were collected from beagle eyes with 10 mm Schirmer tear test strips after insertion of the insert into the canaliculus. Dexamethasone levels in tear fluid were measured by LC-MS/MS. The inserts were prepared according to the same method as described above in Example 1. The exact compositions of the inserts used in the present example are presented in Table 6.

TABLE 6
Compositions of the 0.22, 0.37, 0.46, 0.58, 0.65, 0.72, and 0.85 mg
dexamethasone inserts in percent by weight (% w/w).

| Dose | 0.22 mg | 0.37 mg | 0.46 mg | 0.58 mg |
|---|---|---|---|---|
| Micronized Dexamethasone | 37.1% | 55.0% | 48.0% | 63.8% |
| 4a20kPEG-SG | 55.5% | 39.6% | 45.9% | 31.9% |
| Trilysine Acetate | 1.5% | 1.1% | 1.3% | 0.9% |
| NHS-Fluorescein | 0.5% | 0.4% | 0.0% | 0.3% |
| Sodium Phosphate Dibasic | 4.4% | 3.9% | 4.0% | 2.6% |
| Sodium Phosphate Monobasic | 0.9% | 0.0% | 0.8% | 0.5% |

| Dose | 0.65 mg | 0.72 mg | 0.85 mg |
|---|---|---|---|
| Micronized Dexamethasone | 56.0% | 67.2% | 67.0% |
| 4a20kPEG-SG | 38.8% | 29.0% | 29.1% |
| Trilysine Acetate | 1.1% | 0.8% | 0.8% |
| NHS-Fluorescein | 0.0% | 0.3% | 0.0% |
| Sodium Phosphate Dibasic | 3.4% | 2.3% | 2.6% |
| Sodium Phosphate Monobasic | 0.7% | 0.5% | 0.5% |

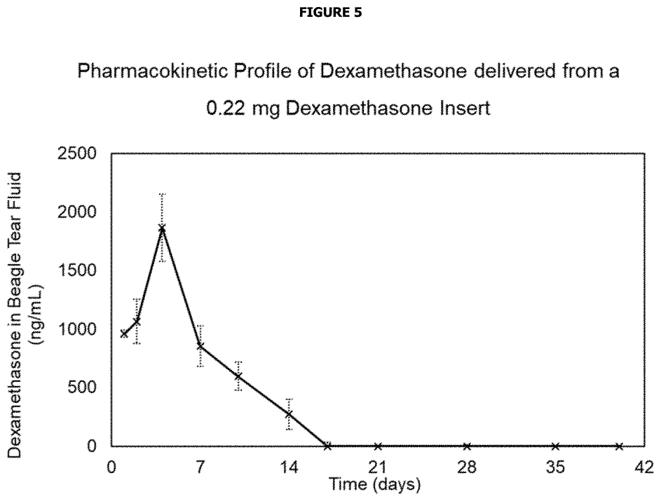

Aqueous humor and/or tear fluid samples were collected at indicated time points and analyzed using LC-MS/MS as described above (Tables 7 and 8; FIG. 5 for the 0.22 mg insert).

TABLE 7
Dexamethasone concentrations in tear fluid of beagle dogs delivered from different doses of
dexamethasone inserts over time (S.D. = standard deviation).

| | Dexamethasone Dose | | | | | | | |
|---|---|---|---|---|---|---|---|---|
| | 0.22 mg | | 0.37 mg | | 0.46 mg | | 0.58 mg | |
| | Average | S.D. | Average | S.D. | Average | S.D. | Average | S.D. |
| Day | (ng/mL) | (ng/mL) | (ng/mL) | (ng/mL) | (ng/mL) | (ng/mL) | (ng/mL) | (ng/mL) |
| 7 | 802 | 486 | 2796 | 1184 | 1040 | 525 | 1822 | 600 |
| 14 | 287 | 342 | 1535 | 750 | 1685 | 902 | 911 | 842 |
| 21 | 2 | 6 | 1146 | 320 | 499 | 418 | 1074 | 559 |
| 28 | 9 | 6 | 190 | 294 | 499 | 608 | 266 | 251 |

| | Dexamethasone Dose | | | | | |
|---|---|---|---|---|---|---|
| | 0.65 mg | | 0.72 mg | | 0.85 mg | |
| | Average | S.D. | Average | S.D. | Average | S.D. |
| Day | (ng/mL) | (ng/mL) | (ng/mL) | (ng/mL) | (ng/mL) | (ng/mL) |
| 0.25 | | | 4534 | 2515 | | |
| 7 | 1119 | 772 | 3418 | 1659 | 737 | 358 |
| 14 | 1905 | 753 | 2538 | 642 | 1162 | 376 |
| 21 | 1070 | 728 | 2114 | 943 | 813 | 481 |
| 28 | 4390 | 806 | 1814 | 642 | 1553 | 733 |
| 35 | | | 1182 | 953 | | |

TABLE 8
Dexamethasone concentrations in aqueous humor of beagle dogs
delivered from different doses of dexamethasone inserts over time.
Dexamethasone Concentration in Beagle Aqueous Humor
(Average ± Standard Deviation; ng/mL)

| | Dexamethasone Dose | | | |
|---|---|---|---|---|
| Day | 0.37 mg | 0.46 mg | 0.65 mg | 0.85 mg |
| 7 | 4.1 ± 2.6 | 7.7 ± 4.8 | 9.7 ± 3.4 | 7.4 ± 1.7 |
| 14 | 7.0 ± 3.9 | 7.9 ± 3.3 | 13.4 ± 1.7 | 13.3 ± 9.8 |
| 21 | 0.7 ± 1.0 | 7.5 ± 2.3 | 9.9 ± 1.9 | 7.7 ± 4.3 |
| 28 | 0.1 ± 0.2 | 1.9 ± 2.0 | 7.2 ± 2.1 | 5.9 ± 1.6 |

Pharmacokinetic results in tear fluid and aqueous humor samples were comparable. The values demonstrate a sustained release of dexamethasone with approximately constant levels of dexamethasone in the tear fluid and aqueous humor for several days depending on the dose, followed by a reduction in released drug amounts (tapering) until ultimately complete release. For instance, the 0.22 mg dexamethasone insert provided approximately constant dexamethasone levels in the tear fluid through 7 days followed by tapering from day 7 on with complete release of dexamethasone from the insert after 17 days following administration, thus resulting in an overall sustained release time of 17 days (FIG. 5). For determining the pharmacokinetic profile in tear fluid of these 0.22 mg inserts as shown in FIG. 5, inserts were placed bilaterally into the punctum of 7 beagles (i.e., a total of 14 eyes) on day 0. Tear fluid samples were collected from beagle eyes with 10 mm Schirmer tear test strips on days 1, 2, 4, 7, 10, 14, 17, 21, 28, 35, 37, and 40 after insertion of the insert into the canaliculus. Dexamethasone levels in tear fluid were measured by LC-MS/MS. Dexamethasone is presented as average values together with corresponding standard deviation error bars. The numbers of samples measured were as follows: For day 1, n was 6 eyes; for day 2, n was 8 eyes; for days 14 and 21, n was 7 eyes; for day 28, n was 6 eyes; for day 35, n was 2 eyes. A single insert delivered dexamethasone to the ocular surface for approximately 14 days, with a sustained level of dexamethasone in the tear fluid maintained through day 7, followed by a tapering from day 7 to day 14 with complete release by day 17. The 0.37 mg dexamethasone insert resulted in constant dexamethasone levels in the tear fluid through 21 days followed by tapering from day 21 through day 28 (Table 7). The tapering was also evident in the aqueous humor in the 0.37 mg dose at day 21 and the 0.46 mg dose at day 28 (Table 8). Of note, the aqueous humor and tear fluid dexamethasone concentrations resulting from the doses tested corresponded to the concentrations achieved by the application of MAXIDEX® eye drops (0.1% dexamethasone suspension) 4 times per day, which contain approximately 50 μg of dexamethasone per drop.

In summary, the dexamethasone concentrations in the aqueous humor of beagle dogs at 7 and 14 days were comparable between all doses tested. In addition, dexamethasone concentrations in tear fluid of beagle dogs were also comparable between all doses tested at 7 days.

Dexamethasone inserts were removed from the canaliculus by manual expression out of the punctum opening at selected time points for a defined number of animals. Remaining dexamethasone was extracted from the inserts and measured by LC-MS/MS as described above. The dexamethasone release rate per day prior to tapering and complete depletion from the insert (as evidenced by a decrease in dexamethasone concentration in tear fluid and/or aqueous humor) was calculated by determining the amount of dexamethasone released from the insert divided by the study day the insert was removed (Table 9). The results demonstrate that the determined dexamethasone release rates per day are comparable between all doses tested. This is in line with the fact that the dexamethasone release rate from an insert according to the present invention is regulated by the drug's solubility in the hydrogel matrix and the tear fluid. Dexamethasone is released from the insert primarily at the interface proximal to the tear fluid, i.e. from the insert portion facing the punctal opening (as exemplarily illustrated in FIG. 6). The released drug levels thus remain largely constant until dexamethasone amounts in the insert are sufficiently reduced at the interface between the insert and the tear fluid, which leads to a gradual tapering effect as observed in the tear fluid and aqueous humor pharmacokinetic profiles. The average amount of dexamethasone released from the inserts according to the invention measured in these studies is essentially independent of the dexamethasone dose and is approximately 0.020 mg per day (or from about 0.015 mg to about 0.025 mg per day) prior to tapering and complete depletion.

TABLE 9
Dexamethasone released per day from dexamethasone inserts
comprising different doses prior to tapering and complete depletion (note
that the two 0.85 mg inserts in the table were two different lots and
measured in two different studies).

| | Average Total Dexamethasone Released Per Day from the | | | | |
|---|---|---|---|---|---|
| | Dexamethasone Inserts | | | | |
| | Dexamethasone Dose | | | | |
| Day | 0.37 mg | 0.46 mg | 0.65 mg | 0.85 mg | 0.85 mg |
| 7 | 0.015 | not tested | not tested | 0.024 | not tested |
| 14 | 0.020 | not tested | not tested | 0.025 | not tested |
| 21 | Tapering/ | 0.015 | 0.018 | 0.022 | 0.020 |
| | Depletion | | | | |
| 28 | Tapering/ | Tapering/ | Tapering/ | 0.019 | 0.017 |
| | Depletion | Depletion | Depletion | | |

The unidirectional drug release into the tear fluid is visually demonstrated exemplarily for the 0.37 mg dexamethasone insert in FIG. 6. Although dexamethasone is released from the inserts prior to (complete) biodegradation of the insert (e.g. for the 0.37 mg dexamethasone insert the drug is completely released after approximately 28 days while the insert has not yet visually degraded to a large extent), extended presence of the drug depleted insert provides the additional longer-term benefit of lacrimal occlusion. In case in certain patients a more prolonged dexamethasone treatment is required or desired, a new insert could be placed on top of the prior, drug-depleted insert (also referred to as “insert stacking”). In any case, due to the insert being biodegradable, there is no need for removal of the insert, which greatly improves patient compliance.

Inserts comprising 0.2mg and 0.3 mg dexamethasone, respectively, are expected to provide an essentially constant concentration of dexamethasone to the ocular surface for a period of up to about 7 days (for the 0.2 mg insert) and up to about 11, or up to about 14 days (for the 0.3 mg insert), after administration. The dexamethasone concentrations will then decrease (taper) over approximately the next 7 days until the active is completely depleted from the 0.2 mg and 0.3 mg dexamethasone insert. A sustained release of therapeutically effective amounts from the inserts according to the present invention is therefore provided for a period of about 14 days and for a period of about 21 days, respectively.

### Safety and Tolerability of Inserts

Potential ocular toxicity, irritation, and systemic exposure were evaluated for a 0.72 mg dexamethasone insert over a 35-day period after intracanalicular insertion in beagle dogs. Reversibility and delayed occurrence of any toxic effects were assessed after a 14-day recovery period.

Two different types of inserts (and each of these both in a version with and a version without dexamethasone contained therein) were evaluated. The first insert type comprised 100% 4-arm 20 k PEG-SG hydrogel material (as described above in Example 1). The second insert type comprised a 50/50 blend of 4-arm 20 k PEG-SG and 4-arm 20 k PEG-SS hydrogel material. Both insert types were prepared according to the same method as described above in Example 1, except that for the second insert type the mentioned PEG precursor blend was used. For the exact composition of the 0.72 mg inserts reference is made to Table 6 (only that 50% of the 4a 20 kPEG-SG in the 0.72 mg insert reported in Table 6 had been replaced by 4a 20 kPEG-SS for those inserts that contained the PEG blend).

The study comprised two groups of beagle dogs. Animals of the first group (n=17) received inserts with dexamethasone, i.e. the first insert type with 100% 4-arm 20 k PEG-SG and dexamethasone in one eye and the second insert type with the 50/50 PEG blend and dexamethasone in the other eye, so each animal received one insert type (with dexamethasone) in each eye, resulting in a total exposure dose of 1.44 mg dexamethasone per animal. Animals of the second group (n=16) received the control inserts (without dexamethasone), i.e. the first insert type with 100% 4-arm 20 k PEG-SG in one eye and the second insert type with the 50/50 PEG blend in the other eye, so each animal received one insert type (without dexamethasone) in each eye.

Evaluations included any observed toxic effects, gross necropsy, and histopathological findings. Ophthalmic examinations included slit lamp biomicroscopy, fluorescein staining, fundoscopy, and tonometry. The slit lamp examination was used to track potential alterations in the cornea, conjunctiva, iris, anterior chamber, and lens. The corneal surface was also assessed using fluorescein stain. The retina was examined for gross changes to the retina or optic nerve and noted as normal or abnormal. Daily clinical and food consumption observations were conducted. Body weight was measured weekly.

In summary, the dexamethasone inserts were well tolerated. Systemically, there were no treatment related effects seen on body weights, food consumption, hematology, clinical chemistry, coagulation, and urinalysis parameters. There were no effects seen in assessments of intraocular pressure and posterior segments of the eyes. Macroscopic and microscopic evaluations showed no test article related findings that would indicate direct test article toxicity. Findings in the punctum were likely due to procedural complication or normal background effects.

Observations from the ophthalmic examinations indicated mild to no irritation, as well as mild conjunctival congestion and discharge, and sluggish to absent pupillary light reflex. Findings were comparable across all groups independent of the type of insert (PEG compositions) and whether dexamethasone was present or not in the insert. The congestion findings were mild and not considered adverse. The discharge was considered to be related to the presence of the punctum plug and not specifically the materials comprising the test article. The sluggish to absent pupillary light reflex observations, which were considered to be due to observational subjectivity were limited and not considered adverse. No delayed occurrence of any toxic effects was observed after the 14-day recovery period.

Plasma concentrations (determined as described above) were below the lower limit of quantification (1.0 ng/mL) for all animals over the study duration, confirming the lack of clinically significant systemic exposure to dexamethasone even at a total dose as high as 1.44 mg per animal (resulting from the two inserts, one insert per eye).

In addition, the presence of the dexamethasone comprising as well as the vehicle control inserts were monitored over the 35-day study duration. For all groups the intracanalicular insert was still present in more than 84% of the animals after the treatment period. However, inserts comprising 100% 4-arm 20 k PEG-SG had a higher overall incidence of insert presence (retention) independent of the presence of dexamethasone or not when compared to the 50/50 PEG blend inserts.

### Example 4: Clinical Trials

The 0.2 mg and 0.3 mg dexamethasone inserts (see Tables 4 and 5 for composition and dimensions of the inserts) were evaluated in a randomized, double-masked, vehicle-controlled phase 2 study to evaluate the efficacy and safety of dexamethasone intracanalicular ophthalmic inserts for the short-term treatment of signs and symptoms of dry eye disease (DED).

The primary objective is photographic assessment of bulbar conjunctival hyperemia change from baseline (CFB) at 15 days (evaluated via central reading center).

The secondary endpoints are severity of Eye Dryness Score (visual analogue scale [VAS]) CFB at 15 days; frequency of Eye Dryness (VAS) CFB at 15 days; severity of Eye Dryness Score (VAS), CFB and absolute values at each post-baseline visit; frequency Eye Dryness Score (VAS), CFB and absolute values at each post-baseline visit; Investigator assessment of bulbar conjunctival hyperemia CFB at each post-baseline visit; Total Corneal Fluorescein Staining (tCFS) [National Eye Institute (NEI) scale], CFB and absolute values at each post-baseline visit; CFS sub-regions using NEI scale; CFB and absolute values at each post-baseline visit; and Ocular Surface Disease Index questionnaire (OSDI©) questionnaire, CFB and absolute values at each post-baseline.

### Efficacy Endpoints

### Primary

Photographic assessment of bulbar conjunctival hyperemia change from baseline (CFB) at 15 days (evaluated via central reading center).

### Secondary

Severity of Eye Dryness Score (visual analogue scale [VAS]) CFB at 15 days

Frequency of Eye Dryness (VAS) CFB at 15 days.

Severity of Eye Dryness Score (VAS), CFB and absolute values at each post-baseline visit.

Frequency Eye Dryness Score (VAS), CFB and absolute values at each post-baseline visit.

Investigator assessment of bulbar conjunctival hyperemia CFB at each post-baseline visit.

Total Corneal Fluorescein Staining (tCFS) [National Eye Institute (NEI) scale], CFB and absolute values at each post-baseline visit.

CFS sub-regions using NEI scale, CFB and absolute values at each post-baseline visit.

Ocular Surface Disease Index questionnaire (OSDI©) questionnaire, CFB and absolute values at each post-baseline visit (total score, each of the three domains, and individual questions).

SPEED questionnaire (overall score and individual questions), CFB at each post-baseline visit.

### Exploratory

Presence of OTX-DED or HV insert at all post-baseline visits

Ease of insertion as assessed by the Investigator

Ease of visualization as assessed by the Investigator

Schirmer Test without anesthesia CFB

Safety Evaluations Adverse Events (Ocular and Non-ocular)

Best-Corrected Visual Acuity (BCVA)

Slit Lamp Biomicroscopy (including punctum assessment)

Intraocular Pressure (IOP)

Fundus Examination

Artificial tear use during the study

### Pharmacokinetics (PK) Parameters

Tear Film PK

Number of Investigational Sites

Approximately 15 sites in the United States (US)

Number of Subjects Planned

Approximately 150 subjects (300 eyes) will be enrolled

Study Population

Subjects with signs and symptoms of DED.

### Study Design and Overview

The study was a randomized, double-masked, vehicle-controlled, phase 2 clinical study designed to evaluate the efficacy and safety, of the inserts for the short-term treatment of subjects with signs and symptoms of DED. Subjects were enrolled into one of three arms as noted in the table below.

| | Duration of IP |
|---|---|
| Arm | Release |
| 0.2 mg Dexamethasone Intracanalicular Opthalmic Insert | 2 weeks |
| 0.3 Dexamethasone Intracanlicular Opthalmic Insert | 3 weeks |
| Hydrogel Vehicle (HV) Intracanalicular Insert | N/A |

Subjects confirmed to be eligible were randomly assigned to one of three treatment groups (0.2 mg, 0.3 mg, or HV) in a 1:1:1 ratio. The dexamethasone insert or HV insert, as applicable, were placed bilaterally, into either the superior or inferior canaliculus (per investigator's preference). The treatment follow-up visits occurred at Week 1/Day 8 (Visit 3), Week 2/Day 15 (Visit 4), Week 3/Day 22 (Visit 5) and Week 4/day 29 (Visit 6). All subjects were be followed until Week 8/Day 57 (Visit 7) to evaluate whether or not the insert was visualized.

Inclusion Criteria was as follows:

1. Provide written informed consent prior to performing any study procedures and are willing to comply with study requirements and the study visit schedule.

2. Are 18 years of age or older.

3. Have a self-reported history or clinically confirmed diagnosis of DED by an eye care professional in both eyes for≥6 months.

4. Have ongoing DED, at screening visit as defined by the following criteria:
a. VAS Eye Dryness severity score≥40.
   And in the same qualifying eye or both eyes:
b. Investigator assessment of bulbar conjunctival hyperemia grade≥2 (CCLRU; 0-4 scale).
c. Unanesthetized Schirmer of >0 and 10 mm.

5. Have corrected visual acuity better than or equal to logarithm of the minimum angle of resolution (log MAR), +0.7 as assessed with Early Treatment of Diabetic Retinopathy Study (ETDRS) charts in both eyes.

6. Are women of childbearing potential (WOCBP) who are non-pregnant, non-lactating, and sexually inactive (abstinent) for 14 days prior to screening and are willing to remain so through the last study visit. Alternatively, WOCBP who are not abstinent must have been using one of the following acceptable methods of birth control for the times specified:
a. Intra-uterine device (IUD) in place for at least 3 months prior to screening and the desire to continue this method through the last study visit.
b. Barrier method (condom or diaphragm) with spermicide for at least 14 days prior to screening and the desire to continue this method through the last study visit.
c. Stable hormonal contraceptive for at least 3 months prior to screening and the desire to continue this method through the last study visit.
d. Surgical sterilization (vasectomy) of partner for at least 6 months prior to Day 1.
e. Alternatively, are post-menopausal women (i.e. no menstrual cycle for at least one year prior to Visit 1/Screening) or are women who have undergone one of the following sterilization procedures at least 6 months prior to screening:
   i. Bilateral tubal ligation
   ii. Hysterectomy
   iii. Bilateral oophorectomy

7. Agree to the removal of non-dissolvable punctal plugs 4 weeks prior to the insertion visit (Day 1, Visit 2) and long-term dissolvable punctal plugs were not placed within 4 months prior to insertion visit (Day 1, Visit 2) and short-term dissolvable punctual plugs were not placed within 6 weeks prior to insertion (Day 1, Visit 2).

8. Agree to restrict the use of artificial tears (AT) through Day 29 of the trial.

### Pre-Procedural Exclusion Criteria

### Subjects were not Eligible for Study Participation if They

1. Have a known or suspected allergy to any component of the study product.

2. Are unwilling to discontinue use of contact lenses for 4 weeks prior to the screening visit and throughout the study period.

3. Have any active systemic disease and/or systemic infection (e.g., fever or current antibiotic use), or uncontrolled medical condition that in the judgment of the investigator could confound study assessments or limit compliance.

4. Have a documented history of ocular allergies, which, in the judgment of the investigator, are likely to have an acute increase in severity during the duration of this trial. Subjects sensitive to seasonal allergens that are not expected to be present during the study are permitted.

5. Have a history of neuropathic pain related to dry eye.

6. Have corneal erosive disease (e.g., multiple filaments, recurrent erosion syndrome) or other conditions suggestive of extensive damage of the cornea.

7. Have a history of glaucoma or ocular hypertension or have intraocular pressure (IOP)<5 mmHg or >24 mmHg or a history of elevated IOP within the past 6 months prior to the screening visit.

8. Have abnormal lid anatomy that may confound study data, in the judgement of the investigator.

9. Have a diagnosis of any of the following:
a. Active ocular infection
b. In the judgement of the investigator uncontrolled anterior blepharitis or posterior blepharitis or blepharitis requiring the use of systemic or antibiotic therapy.
c. Uveitis
d. Moderate to severe pinguecula or pterygia, in the judgement of the investigator
e. Stevens-Johnson syndrome
f. Mucous membrane pemphigoid
g. Significant conjunctival scarring, in the judgement of the investigator
h. Chemical burn
i. Herpetic or neurotrophic keratitis
j. Congenitally absent lacrimal gland or meibomian glands
k. Nasolacrimal duct obstruction in either eye

10. Have had penetrating intraocular surgery within 6 months or require penetrating intraocular surgery during the study, eyelid surgery within 6 months, corneal laser refractive surgery within the last 1 year, cauterization of the punctum resulting in complete occlusion of both punctum in both eyes, glaucoma surgery or corneal transplantation (full thickness, anterior or posterior).

11. Have taken any of the following in either eye within 30 days prior to the screening visit:
a. Topical ocular corticosteroids
b. Topical ocular antibiotics
c. Topical ocular NSAID
d. Topical ocular antihistamines and/or mast cell stabilizers
e. Topical ocular or nasal vasoconstrictors
f. Topical ocular cyclosporine (e.g., Cequa®, Restasis®)
g. Lifitegrast (Xiidra®)
h. Autologous tears
i. Intranasal Tear Neurostimulation

12. Have taken any of the following in either eye prior to the screening visit:
a. Periocular injection of any corticosteroid solution—3 months
b. Corticosteroid intra-vitreal depot injection—3 months
c. Ozurdex—6 months
d. Retisert—40 months

13. Have altered the dose of the following within 30 days prior to the screening visit (i.e., should keep dose stable throughout the study):
a. Nutraceuticals or multivitamins
b. Tetracycline compounds (tetracycline, doxycycline, or minocycline)
c. Inhaled, intramuscular or intra-articular corticosteroids (mouth or nasal spray form), or dermatological steroids (use is allowed if applied to <4 locations for <4 continuous days)

14. Have altered the dose of the following within 6 months prior to the screening visit (i.e., should keep dose stable throughout the study):
a. Systemic anticholinergics
b. Antidepressants (with the exception of rare usage as a sleep aid)
c. Oral corticosteroids (e.g., prednisone. Prednisone dose must be less than 11 mg/day)

15. Have taken isotretinoin (Accutane) or systemic immunosuppressive agents within 6 months prior to the screening visit.

16. Have participated in any other investigational study within 30 days of the screening visit or plans to participate in any other investigational study during the follow-up period.

17. Are an employee of the site or an immediate family member of an employee of the site.

18. Are a current smoker (including marijuana, cigar, cigarette, and/or e-cigarettes).

19. Have a known history of alcohol and/or drug abuse or are currently using illicit drugs or plan to use illicit drugs for the duration of the study. Recreational or medicinal marijuana allowed if oral consumption (no inhaled use).

20. Are unwilling or unable to comply with the study protocol.

21. The Investigator determines that the subject should not be included for reasons not already specified (e.g., systemic, behavioral, or other ocular disease/abnormality) if the health of the subject or the validity of the study outcomes may be compromised by the subject's enrollment.

### Entry/Randomization Criteria

To qualify for insertion at Day 1 (Visit 2), a subject continued to meet all screening inclusion/exclusion criteria with the following exceptions/additions:

1. VAS Eye Dryness severity score≥35.

2. Investigator assessment of bulbar conjunctival hyperemia grade≥2 (CCLRU; 0-4 scale).

3. Subjects must not have taken prohibited medications and have completed the appropriate washout of prior medications, if necessary.

4. Subjects who require AT use must not have administered>3 time/day for 3 consecutive days during the washout period.

NOTE: AT use is not permitted unless absolutely necessary. If subject requires AT use and has administered>3 times/for 3 days during the Screening period, they are not eligible for randomization.

### Procedural Exclusion Criteria

Subjects were considered procedural screen failures if the investigator were unsuccessful at placing the dexamethasone insert or HV Intracanalicular Ophthalmic insert in both eyes (i.e., neither eye has an insert). Subject was followed per protocol if the investigator successfully placed one insert.

### Artificial Tear Use

Preservative-free AT was provided only if absolutely required. If AT was administered, subjects were instructed not to administer AT within 2 hours prior to any study visit. Preservative-free AT use if needed was recorded by subjects in a daily diary.

### Sample Size Considerations

This study was not powered to show statistical significance but provided initial estimates and trends of the endpoints for use in future trial designs. Statistical analyses was descriptive.

### Statistical Methods

Summaries for continuous variables included the sample size, mean, standard deviation, median, minimum, and maximum. Summaries for discrete variables included frequencies and percentages. The baseline visit was defined as the last non-missing measure prior to initiation of IP. Differences between treatment groups was calculated as dexamethasone inserts minus HV and change from baseline will be calculated as follow-up visit minus baseline visit values.

The study eye was the eye with the higher grade from the photographic assessment of bulbar conjunctival hyperemia, conducted by the central reading center, was designated as the study eye and the other eye designated as the non-study eye. If both eyes had the same bulbar conjunctival hyperemia grades, the right eye was the study eye.

Efficacy and safety summaries were presented for the study eye. Safety summaries of the non-study eye were also be included, and additional analyses was presented for the non-study eye. All summaries were presented by treatment group and visit, where appropriate. In addition to looking at the individual formulations, the two formulations of dexamethasone inserts were combined and summarized

### Primary Hypothesis

Statistical hypothesis for the primary efficacy endpoint of photographic assessment of bulbar conjunctival hyperemia at 15 days was as follows: H10: There is no difference in mean change from baseline in photographic assessment of bulbar conjunctival hyperemia grades between dexamethasone inserts and HV treated subjects at 15 days.

H1a: There is a difference in mean change from baseline in photographic assessment of bulbar conjunctival hyperemia grades between dexamethasone inserts and HV treated subjects at 15 days.

### Primary Efficacy Analyses

For the primary efficacy endpoint analyses of change from baseline in photographic assessment of bulbar conjunctival hyperemia (evaluated by central reading center) worst zone in the study eye at Visit4 (Day 15) (as defined below), the following modeling and analyses was performed on mITT population, observed data:

An analysis of covariance (ANCOVA) model was run to estimate least square (LS) means treatment. This model included the baseline value as a covariate for adjustment and treatment group as the sole factor. Least square means was used to make treatment comparisons using Dunnett's adjustment for multiple comparisons to a control. Statistical significance of treatment differences was determined using a two-sided significance level of α=0.05.

In the ANCOVA model, LS means treatment group comparison of 0.2 mg dexamethasone insert to HV, 0.3 mg dexamethasone insert to HV, and Overall dexamethasone insert (combined 0.2 mg and 0.3 mg using sample size weights of 0.2 mg and 0.3 mg groups as coefficients for the parameters) to HV was be reported. Unadjusted treatment group analyses using two-sample t-test and Wilcoxon rank sum test was run on these treatment group combinations.

Each bulbar conjunctival hyperemia zone (nasal, temporal, and frontal) and the Total of the three zones in study eye was analyzed in a manner similar to the primary efficacy variable as described above.

The same analyses was done for the non-study eye. For non-study eye, the worst zone similar to worst zone in study eye (details below) was independently determined using photographic assessment bulbar conjunctival hyperemia in non-study eye at baseline. The primary efficacy endpoints was also analyzed on PP population.

The primary analysis was based on the change from baseline in worst zone of photographic assessment of bulbar conjunctival hyperemia at baseline in the study eye. The worst (highest grading score) zone was determined among Nasal, Temporal, and Frontal zones of photographic assessment of bulbar conjunctival hyperemia; if there was equal scores in two or more zones, the worst zone was selected in a preferential order with Nasal zone being the most preferred, followed by Temporal zone, and then by Frontal zone in study eye at baseline. Change from baseline in this same chosen zone at Visit 4 (Day 15) was investigated.

Intent-to-Treat (ITT): The ITT population included all randomized subjects. Subjects who were randomized but not treated due to procedural screen failures were included as part of ITT population.

Modified Intent-to-Treat (mITT): The Modified ITT included all subjects in ITT who received the insert in the study eye, investigational product (IP) (OTX-DED or HV). The mITT population was used as the primary efficacy analysis population and was also used for all efficacy endpoints. Analyses performed on the mITT population was according to the treatment the subject was randomized.

Per Protocol (PP): The PP population included all mITT subjects who did not have any major protocol deviation and who did not report excessive artificial tear use defined as greater or equal to 30 drops between Insertion/Day 1 (Visit 2) and Week 2 (Visit 4). Analysis on the PP population was used as secondary efficacy analysis and was performed for select efficacy endpoints, analyzing subjects under the treatment actually received. Important protocol deviations were identified prior to locking the study database.

Safety: The Safety population included all randomized subjects who received Investigational Product (IP) (OTX-DED or HV). Analyses performed on the Safety population was according to the treatment the subject actually received

### Sensitivity Analyses

To determine robustness of results, the sensitivity analyses was performed for the primary efficacy endpoint, bulbar conjunctival hyperemia at Visit4 (Day 15), on the mITT and PP populations by using:

1. MCMC MI

2. last observation carried forward (LOCF)

3. FCS MI.

As sensitivity analyses, LOCF was analyzed in a manner similar to the primary efficacy variable (details above). MCMC MI and FCS MI was analyzed in a manner similar to the primary efficacy variable (details above), however, for treatment comparisons, least square means was not adjusted for multiple comparison and for the unadjusted treatment group comparisons, only t-test was used

### Secondary Efficacy Analyses

The below secondary efficacy endpoints were analyzed in a manner similar to the primary efficacy variable using ANCOVA model of change from baseline using observed data:

1. Severity of eye dryness score at Visit 4 (Day 15) and other post-baseline visits,

2. Frequency of eye dryness score at Visit 4 (Day 15) and other post-baseline visits,

3. Investigator assessment of bulbar conjunctival hyperemia in worst zone, Nasal, Temporal, Frontal zones, and Total at each post-baseline visit,

4. Total corneal fluorescein staining (tCFS) and CFS sub-regions at each post-baseline visit.

In addition, sensitivity analyses done for the primary efficacy endpoint (see above) was also performed on severity and frequency of eye dryness scores, investigator assessment of bulbar conjunctival hyperemia, tCFS, and CFS sub-regions endpoints.
The below secondary efficacy endpoints were analyzed using ANCOVA model of change from baseline at each post-baseline visit similar to primary efficacy endpoint analysis (see above) using observed data:

5. Ocular Surface Disease Index questionnaire (OSDI©) at each post-baseline visit (total score, each of the three domains, and individual questions) and

6. SPEED questionnaire (overall score and individual questions), CFB at each post-baseline visit will be analyzed.

All the secondary endpoints were analyzed on mITT and PP populations.

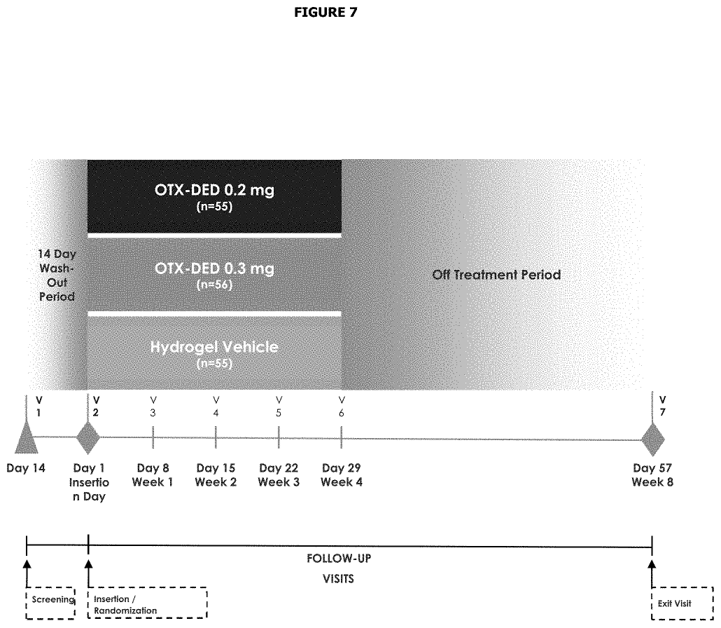

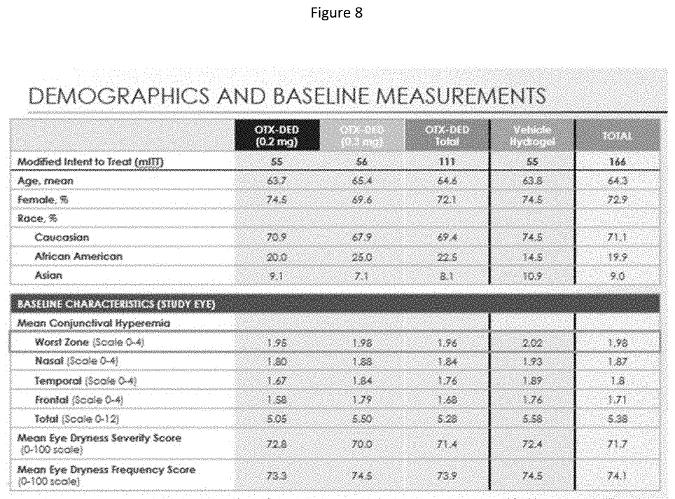

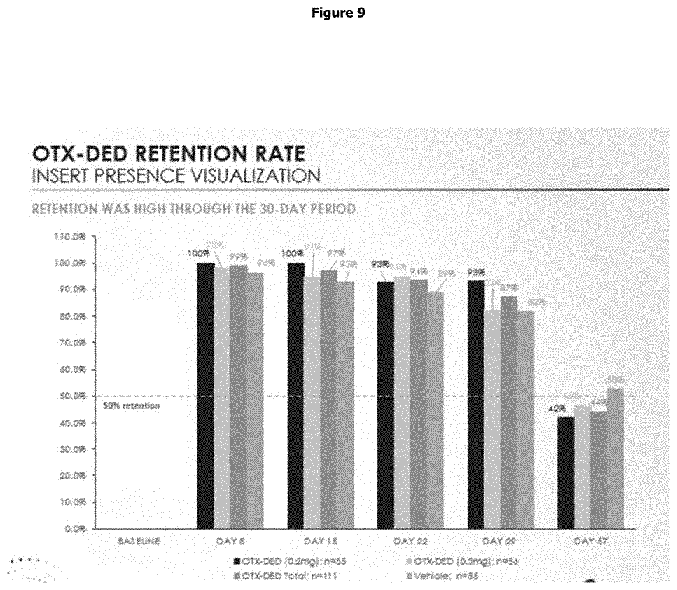

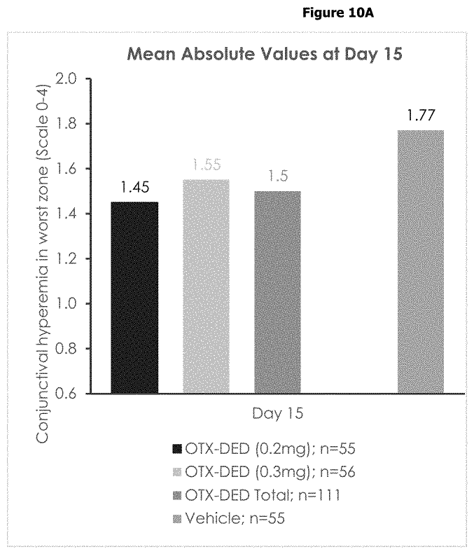

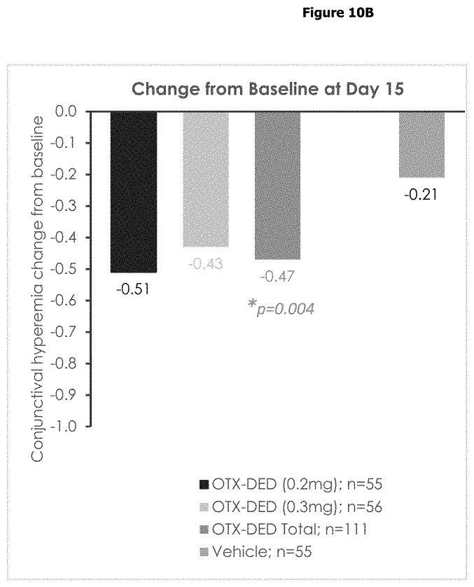

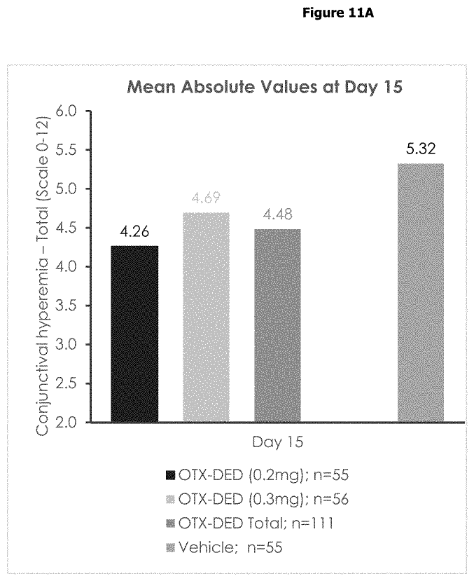

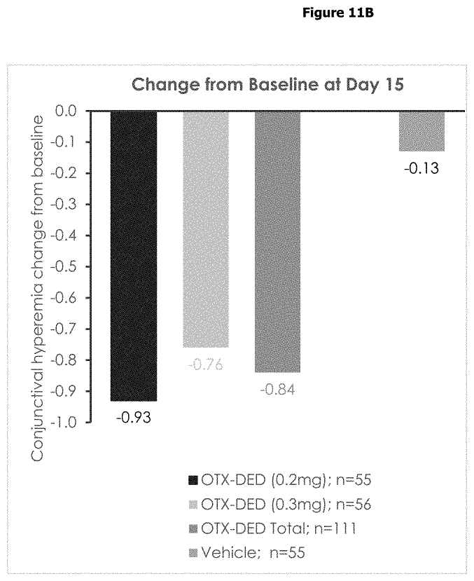

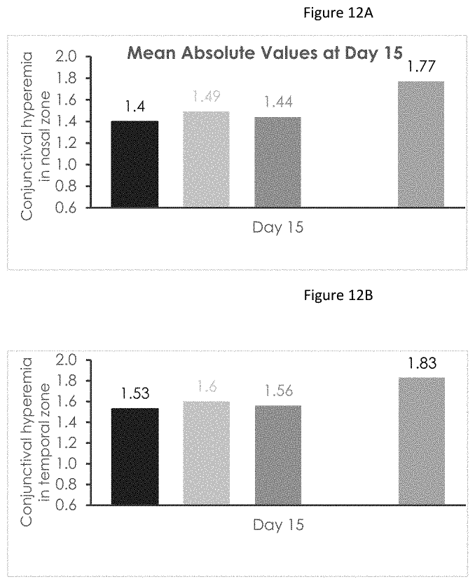

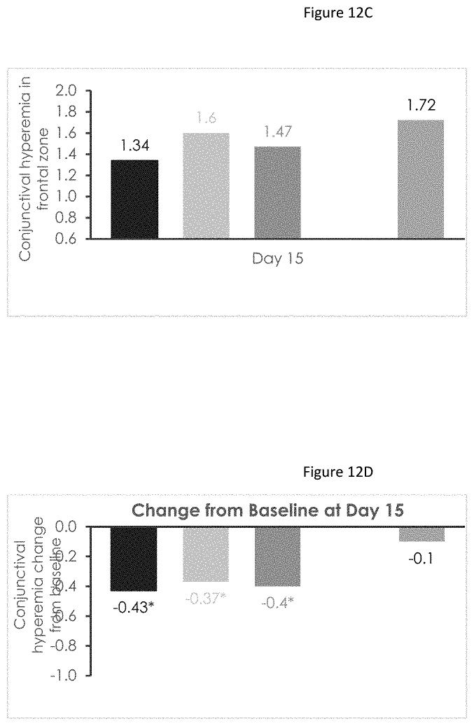

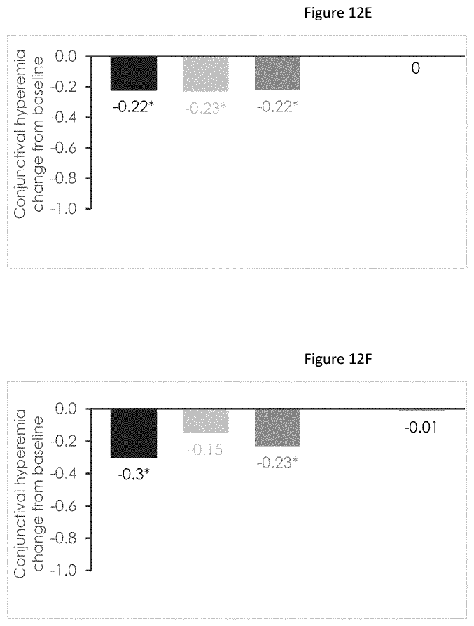

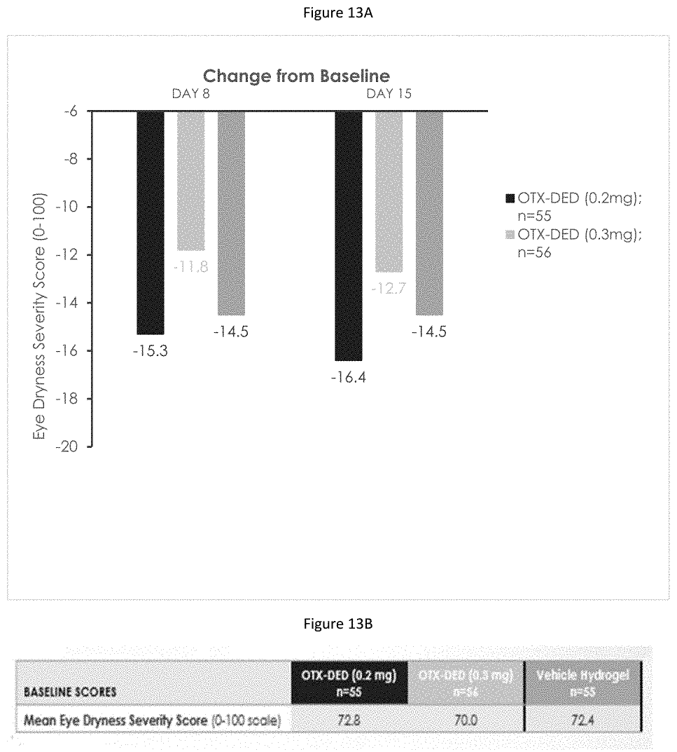

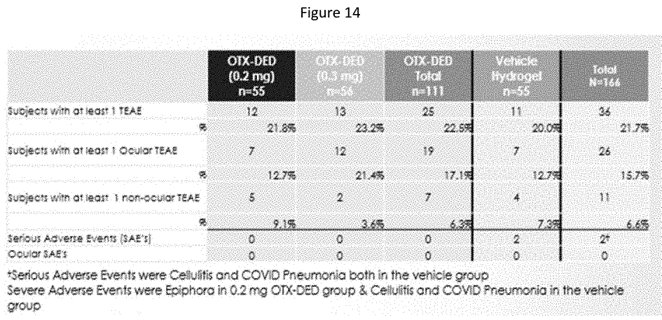

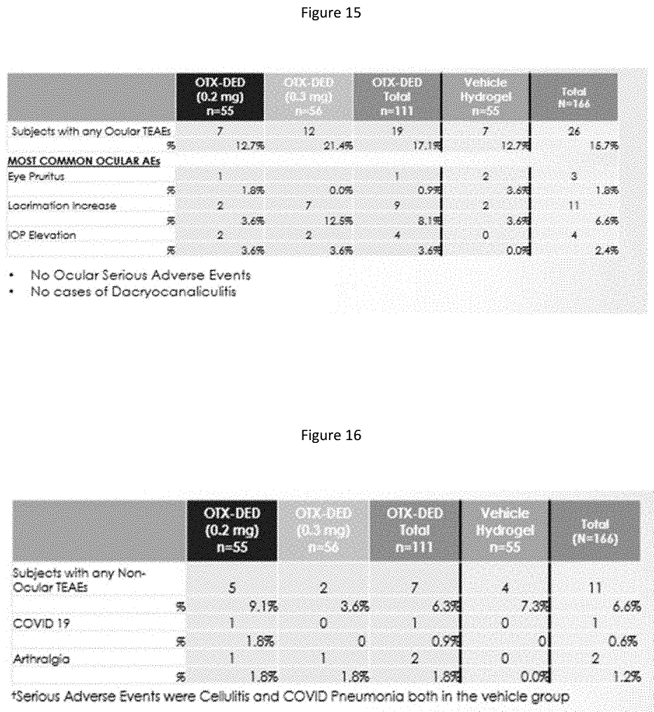

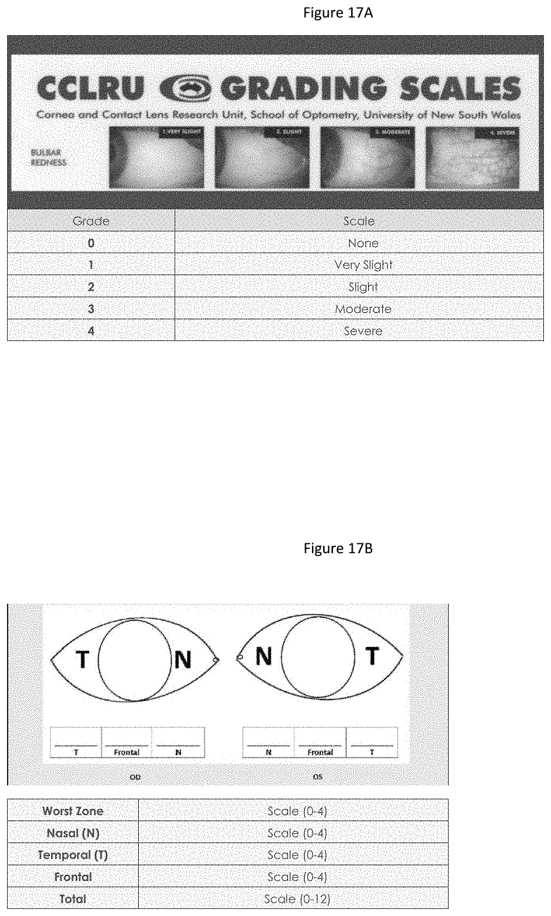

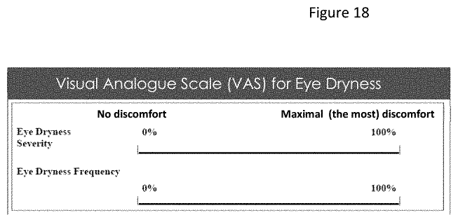

The Phase 2 clinical study is outlined in FIG. 7

### Results

The clinical trial enrolled 166 subjects in the modified intent-to-treat (ITT) population which included subjects who were randomized and had the dexamethasone inserts placed in the study eye. The clinical trial achieved its pre-specified primary endpoint, demonstrating a statistically significant change of bulbar conjunctival hyperemia from baseline to day 15 compared to vehicle hydrogel using a central reading photographic assessment in the modified ITT population. Change from baseline using the CCLRU Grading scale (0-4) was −0.51 for the 0.2 mg group (n=55), −0.43 for the 0.3 mg group (n=56), and −0.21 for the vehicle hydrogel insert group (n=55). These differences were statistically significant compared with the vehicle hydrogel for both the 0.2 mg group (p=0.004) and the 0.3 mg group (p=0.028). Sensitivity analysis using different methods of imputation including last observation carry forward (LOCF), Markov Chain Monte Carlo (MCMC), and fully conditional specification (FCS) were consistent with the primary analysis. Improvements from baseline were noted in the VAS dry eye symptoms for both 0.2 mg and 0.3 mg groups, but there was little separation between and the vehicle hydrogel insert.

Dexamethasone inserts (both formulations) were observed to have a favorable safety profile and were generally well tolerated. There were two non-ocular serious adverse events, both in the vehicle hydrogel insert group, which were evaluated to be not related. There were no ocular serious adverse events. The most common ocular adverse events for subjects treated with the dexamethasone inserts were epiphora (lacrimation increase) (8.1%) and elevated intraocular pressure (IOP) (3.6%). All other ocular adverse events occurred in less than 1% of subjects. The most common non-ocular adverse event for subjects treated with the dexamethasone inserts was arthralgia (joint pain) which was seen in 1.8% of subjects. All other non-ocular adverse events occurred in less than 1% of subjects.

Statistically significant improvement in the primary endpoint (bulbar conjunctival hyperemia in the worst zone) for dexamethasone inserts relative to vehicle hydrogel for 0.2 and 0.3 mg groups

Trial not powered for statistical significance

Data for secondary endpoints of conjunctival hyperemia scores best for Total=Nasal>Temporal>Frontal

All statistically significant except for Frontal (0.3 mg group)

Sensitivity analysis (MCMC, LOCF, FCS) shows similar results as expected due to minimal data missing (only about 3%)

Both doses seem to perform well with no dose response seen

Symptoms (eye dryness score) improved from baseline in all three groups, with no separation between active groups and vehicle

Preliminary outlier analysis and post-hoc analysis show potential opportunities to differentiate between dexamethasone inserts and vehicle hydrogel groups

Observed to have a favorable safety profile and were generally well tolerated, with low rates of ocular pain/discomfort/irritation

Most common adverse events in dexamethasone insert treated groups (0.2 & 0.3 mg)—epiphora (lacrimation increase) (8.1%), IOP elevation (3.6%)

No ocular serious adverse events (SAE's)

1. A sustained release biodegradable ocular insert comprising a hydrogel and a glucocorticoid, wherein glucocorticoid particles are dispersed within the hydrogel, and wherein the insert in its dry state has a length of less than about 2.75 mm.

2. The sustained release biodegradable ocular insert of claim 1, wherein the insert contains less than about 375 μg dexamethasone or an equivalent dose of another glucocorticoid.

3. The sustained release biodegradable insert of claim 1, wherein the insert provides for a release of a therapeutically effective amount of the glucocorticoid for a period of up to about 1 month after administration.

4. The sustained release biodegradable ocular insert of claim 1, wherein the glucocorticoid is dexamethasone.

5. The sustained release biodegradable ocular insert of claim 1, wherein the insert is an intracanalicular insert.

6. The sustained release biodegradable ocular insert of claim 1, comprising from about 160 μg to about 250 μg dexamethasone.

7. The sustained release biodegradable ocular insert of claim 6, comprising from about 180 μg to about 220 μg dexamethasone,

8. The sustained release biodegradable ocular insert of claim 7, comprising about 200 μg dexamethasone.

9. The sustained release biodegradable ocular insert of claim 1, comprising from about 240 μg to about 375 μg dexamethasone.

10. The sustained release biodegradable ocular insert of claim 9, comprising from about 270 μg to about 330 μg dexamethasone.

11. The sustained release biodegradable ocular insert of claim 10, comprising about 300 μg dexamethasone.

12. The sustained release biodegradable ocular insert of claim 1, wherein the insert is cylindrical or essentially cylindrical.

13. The sustained release biodegradable ocular insert of claim 1, wherein the insert is non-cylindrical.

14. The sustained release biodegradable ocular insert of claim 1, wherein the insert is cylindrical or essentially cylindrical and in its dry state has a length of less than about 2.5 mm.

15. The sustained release biodegradable ocular insert of claim 1, wherein the insert is cylindrical or essentially cylindrical and in its dry state has a diameter of less than about 0.75 mm.

16. The sustained release biodegradable ocular insert of claim 1, wherein the insert is cylindrical or essentially cylindrical and in its dry state has a length of about 2.14 mm to about 2.36 mm and a diameter of about 0.41 mm to about 0.55 mm.

17. The sustained release biodegradable ocular insert of claim 1, wherein the insert is cylindrical or essentially cylindrical and upon hydration (after 24 hours in phosphate-buffered saline at a pH of 7.2 at 37° C.) the diameter of the insert is increased and the length of the insert is decreased.

18. The sustained release biodegradable ocular insert of claim 17, wherein the ratio of the diameter of the insert in the hydrated state to the diameter of the insert in the dry state is in the range of about 1.5 to about 4.

19. The sustained release biodegradable ocular insert of claim 18, wherein the ratio of the diameter of the insert in the hydrated state to the diameter of the insert in the dry state is in the range of about 2 to about 3.5.

20. The sustained release biodegradable ocular insert of claim 17, wherein the ratio of the length of the insert in the hydrated state to the length of the insert in the dry state is about 0.9 or less.

21-141. (canceled)
